SEQUENCE LISTING (46 sequences)

SEQ ID NO: 1 - DNA, 1997 bp, Homo sapiens
cccgccccgcctctccaaaaagctacaccgacgcggaccgcggcggcgtcctccctcgccctcgcttcacctcgcgggctccgaatgcggggagctcggatgtccggtttcctgtgaggcttttacctgacacccgccgcctttccccggcactggctgggagggcgccctgcaaagttgggaacgcggagccccggacccgctcccgccgcctccggctcgcccagggggggtcgccgggaggagcccgggggagagggaccaggaggggcccgcggcctcgcaggggcgcccgcgcccccacccctgcccccgccagcggaccggtcccccacccccggtccttccaccatgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaagattgtactgttttccagttcatcgattttctattatggaaaactgtgttgccacagtagaactgtctgtgaacagagagacccttgtgggtccatgctaacaaagacaaaagtctgtctttcctgaaccatgtggataactttacagaaatggactggagctcatctgcaaaaggcctcttgtaaagactggttttctgccaatgaccaaacagccaagattttcctcttgtgatttctttaaaagaatgactatataatttatttccactaaaaatattgtttctgcattcatttttatagcaacaacaattggtaaaactcactgtgatcaatatttttatatcatgcaaaatatgtttaaaataaaatgaaaattgtattat

SEQ ID NO: 2 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 3 - DNA, 2781 bp, Homo sapiens
atggatatgtttcctctcacctgggttttcttagccctctacttttcaagacaccaagtgagaggccaaccagacccaccgtgcggaggtcgtttgaattccaaagatgctggctatatcacctctcccggttacccccaggactacccctcccaccagaactgcgagtggattgtttacgcccccgaacccaaccagaagattgtcctcaacttcaaccctcactttgaaatcgagaagcacgactgcaagtatgactttatcgagattcgggatggggacagtgaatccgcagacctcctgggcaaacactgtgggaacatcgccccgcccaccatcatctcctcgggctccatgctctacatcaagttcacctccgactacgcccggcagggggcaggcttctctctgcgctacgagatcttcaagacaggctctgaagattgctcaaaaaacttcacaagccccaacgggaccatcgaatctcctgggtttcctgagaagtatccacacaacttggactgcacctttaccatcctggccaaacccaagatggagatcatcctgcagttcctgatctttgacctggagcatgaccctttgcaggtgggagagggggactgcaagtacgattggctggacatctgggatggcattccacatgttggccccctgattggcaagtactgtgggaccaaaacaccctctgaacttcgttcatcgacggggatcctctccctgacctttcacacggacatggcggtggccaaggatggcttctctgcgcgttactacctggtccaccaagagccactagagaactttcagtgcaatgttcctctgggcatggagtctggccggattgctaatgaacagatcagtgcctcatctacctactctgatgggaggtggacccctcaacaaagccggctccatggtgatgacaatggctggacccccaacttggattccaacaaggagtatctccaggtggacctgcgctttttaaccatgctcacggccatcgcaacacagggagcgatttccagggaaacacagaatggctactacgtcaaatcctacaagctggaagtcagcactaatggagaggactggatggtgtaccggcatggcaaaaaccacaaggtatttcaagccaacaacgatgcaactgaggtggttctgaacaagctccacgctccactgctgacaaggtttgttagaatccgccctcagacctggcactcaggtatcgccctccggctggagctcttcggctgccgggtcacagatgctccctgctccaacatgctggggatgctctcaggcctcattgcagactcccagatctccgcctcttccacccaggaatacctctggagccccagtgcagcccgcctggtcagcagccgctcgggctggttccctcgaatccctcaggcccagcccggtgaggagtggcttcaggtagatctgggaacacccaagacagtgaaaggtgtcatcatccagggagcccgcggaggagacagtatcactgctgtggaagccagagcatttgtgcgcaagttcaaagtctcctacagcctaaacggcaaggactgggaatacattcaggaccccaggacccagcagccaaagctgttcgaagggaacatgcactatgacacccctgacatccgaaggtttgaccccattccggcacagtatgtgcgggtatacccggagaggtggtcgccggcggggattgggatgcggctggaggtgctgggctgtgactggacagactccaagcccacggtaaaaacgctgggacccactgtgaagagcgaagagacaaccaccccctaccccaccgaagaggaggccacagagtgtggggagaactgcagctttgaggatgacaaagatttgcagctcccttcgggattcaattgcaacttcgatttcctcgaggagccctgtggttggatgtatgaccatgccaagtggctccggaccacctgggccagcagctccagcccaaacgaccggacgtttccagatgacaggaatttcttgcggctgcagagtgacagccagagagagggccagtatgcccggctcatcagcccccctgtccacctgccccgaagcccggtgtgcatggagttccagtaccaggccacgggcggccgcggggtggcgctgcaggtggtgcgggaagccagccaggagagcaagttgctgtgggtcatccgtgaggaccagggcggcgagtggaagcacgggcggatcatcctgcccagctacgacatggagtaccagattgtgttcgagggagtgatagggaaaggacgttccggagagattgccattgatgacattcggataagcactgatgtcccactggagaactgcatggaacccatctcggcttttgcagtggacatcccagaaatacatgagagagaaggatatgaagatgaaattgatgatgaatacgaggtggactggagcaattcttcttctgcaacctcagggtctggcgccccctcgaccgacaaagaaaagagctggctgtacaccctggatcccatcctcatcaccatcatcgccatgagctcactgggcgtcctcctgggggccacctgtgcaggcctcctgctctactgcacctgttcctactcgggcctgagctcccgaagctgcaccacactggagaactacaacttcgagctctacgatggccttaagcacaaggtcaagatgaaccaccaaaagtgctgctccgaggcatga

SEQ ID NO: 4 - protein, 926 aa, Homo sapiens
MDMFPLTWVFLALYFSRHQVRGQPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVKTLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAVDIPEIHEREGYEDEIDDEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 5 - DNA, 3540 bp, Homo sapiens
ggagggctggggcggtacgaagcgggggtgggctctgcgcgtaatggcagcgccgtggcctcgcgtccatctttgccgttctctcggacctgtcacaaaggagtcgcgccgccgccgccgccccctccctccggtgggcccgggaggtagagaaaaactgccattggatgtccagaatcccctgtagttgataatgttgggaataagctctgcaactttctttggcattcagttgttaaaaacaaataggatgcaaattcctcaactccaggttatgaaaacagtacttggaaaactgaaaactacctaaatgatcgtctttggttgggccgtgttcttagcgagcagaagccttggccagggtctgttgttgactctcgaagagcacatagcccacttcctagggactggaggtgccgctactaccatgggtaattcctgtatctgccgagatgacagtggaacagatgacagtgttgacacccaacagcaacaggccgagaacagtgcagtacccactgctgacacaaggagccaaccacgggaccctgttcggccaccaaggaggggccgaggacctcatgagccaaggagaaagaaacaaaatgtggatgggctagtgttggacacactggcagtaatacggactcttgtagataatgatcaggaacctccctattcaatgataacattacacgaaatggcagaaacagatgaaggatggttggatgttgtccagtctttaattagagttattccactggaagatccactgggaccagctgttataacattgttactagatgaatgtccattgcccactaaagatgcactccagaaattgactgaaattctcaatttaaatggagaagtagcttgccaggactcaagccatcctgccaaacacaggaacacatctgcagtcctaggctgcttggccgagaaactagcaggtcctgcaagtataggtttacttagcccaggaatactggaatacttgctacagtgtctgaagttacagtcccaccccacagtcatgctttttgcacttatcgcactggaaaagtttgcacagacaagtgaaaataaattgactatttctgaatccagtattagtgaccggcttgtcacattggagtcctgggctaatgatcctgattatctgaaacgtcaagttggtttctgtgcccagtggagcttagacaatctctttttaaaagaaggtagacagctgacctatgagaaagtgaacttgagtagcattagggccatgctgaatagcaatgatgtcagcgagtacctgaagatctcacctcatggcttagaggctcgctgtgatgcctcctcttttgaaagtgtgcgttgcaccttttgtgtggatgccggggtatggtactatgaagtaacagtggtcacttctggcgtcatgcagattggctgggccactcgagacagcaaattcctcaatcatgaaggctacggcattggggatgatgaatactcctgtgcgtatgatggctgccggcagctgatttggtacaatgccagaagtaagcctcacatacacccatgctggaaagaaggagatacagtaggatttctgttagacttgaatgaaaagcaaatgatcttctttttaaatggcaaccagctgcctcctgaaaagcaagtcttttcatctactgtatctggattttttgctgcagctagtttcatgtcatatcaacaatgtgagttcaattttggagcaaaaccattcaaatacccaccatctatgaaatttagcacttttaatgactacgccttcctaacagctgaagaaaaaatcattttgccaaggcacaggcgtcttgctctgttgaagcaagtcagtatccgagaaaactgctgttccctttgttgtgatgaggtagcagacacacaattgaagccatgtggacacagtgacctgtgcatggattgtgccttgcagctggagacctgcccattgtgtcgtaaagaaatagtatctagaatcagacagatttctcatatttcatgacacatgtgaagaggcatcgtggacttttttctactcaattccagccaatgttgaaaagaaaaagaaaaaaaaaactctctaatcagttgtacacacattgaaacttatagccatggccagattttatgctaaaaatggtagtttgtcaaagacaaaattctcttagaatctaatccaacttgccagccctgagaaaatcccttttaaggccaaggaaagctgaatgctagcagccaggcctgtggtacttccatgagaaaccatagcagacaatgccctcccaagtactgaaatcacactggaatcccccttgttgggttcatttgattgtttaacacaggatgtgttgtgtcattctgaagtttttatttggggcagaagtctttatggagatgtaaatgacagcgtttctgggttatgcataacttctcactggtcagagacaccggtgtgtcaagcatggatattgcattgcaagacttgaatctataaaaattagaatcacacagtcagtactacaagcaaaacagagaacctgaaagaaggtgcacagactgtaagaaaaaacccaagtttgtgatatttcagtgattccaaagaacattctaggttttttgtttgtttttttgttttttgggttttttttttttactgcagaaaattggtggtattttcacattcatagtgtttctatccaatttcagtacccacatttaatgaggaaaaaatgttttaccaatgaaggaggaattcttaaattagctgtaatgttaggttggagaaaatttggtatttagggtattttcaaggtaccatcaaatcagatttctgtttttttgttaaaaaaaatttttttaatcagtattgtttttacaagtaatatactttgaaactcttgaactaatagtctcaaaaactctagaggacagtctgagaacacgtatttctattgttctaaataaatacatgtttttgaatagttcaatcatgaattattgactatgtcttcatcaaaagtgttaatccctctcagggtctctggtgaagaccttcaagagtttggttttttctcccaggaaattggaaggtagaattgtaaattcatagaacttcttttataatggtgtacctcagcagctgcctttcaatttatgccaagtccttacagagtttatacttgaatagtaaatatgtcttctgagttttacagtgtcttaaactcaatgcacatttttttttcttctttttccaccccttcttgtttgtagttcattatacctgtcctattacagaactgatttccttcctggctgtacatgttggggtgctggatttttttccgtgtctttagtcttccataaatccacacacacacacacacacaaaaaatatatatatatataaatatatatgtaggatacatgttctcttctttagcttgtggtgaatacagtaatttgcattgaagaataaaacatctgttgccttttttgactaag

SEQ ID NO: 6 - protein, 576 aa, Homo sapiens
MIVFGWAVFLASRSLGQGLLLTLEEHIAHFLGTGGAATTMGNSCICRDDSGTDDSVDTQQQQAENSAVPTADTRSQPRDPVRPPRRGRGPHEPRRKKQNVDGLVLDTLAVIRTLVDNDQEPPYSMITLHEMAETDEGWLDVVQSLIRVIPLEDPLGPAVITLLLDECPLPTKDALQKLTEILNLNGEVACQDSSHPAKHRNTSAVLGCLAEKLAGPASIGLLSPGILEYLLQCLKLQSHPTVMLFALIALEKFAQTSENKLTISESSISDRLVTLESWANDPDYLKRQVGFCAQWSLDNLFLKEGRQLTYEKVNLSSIRAMLNSNDVSEYLKISPHGLEARCDASSFESVRCTFCVDAGVWYYEVTVVTSGVMQIGWATRDSKFLNHEGYGIGDDEYSCAYDGCRQLIWYNARSKPHIHPCWKEGDTVGFLLDLNEKQMIFFLNGNQLPPEKQVFSSTVSGFFAAASFMSYQQCEFNFGAKPFKYPPSMKFSTFNDYAFLTAEEKIILPRHRRLALLKQVSIRENCCSLCCDEVADTQLKPCGHSDLCMDCALQLETCPLCRKEIVSRIRQISHIS

SEQ ID NO: 7 - DNA, 2589 bp, Homo sapiens
ttggagacggagtctcactctgtcacccattctggagtacaatggcatgatctcagctcactgcaacctcctccttctgggttcaagtgattcttctgcctcagcctcctgagtagctgggactacaggcttgtgccaccacactcagctaatttttgtattttcagtagagacggggtctccccatattggccaggctggtcttgaactcctgacctcaaacgatctgcccaccccagcctcccaaagtgctgggattacaagtgtgagccaccacacccagcctaatgtaactgttttcctttccttttagtctttatctcattgttatatgtgattataatgttgtcatttatgcattcacatcaccagtaattatctcaaacagatcttaaatgctgcctatcatgttgaagtcacgtttcactcagggtcgtcagttacaaggatgctatgggagcaaatcaaacagatcatccagcgcatcacatgggtgagcccacctgccatcactctggaatggaagaggaaggtggcccaggaagccattgagagcctcagcgcctccaaattggctaagagcatttgcagccaattccggactcggctcaatagttcccacgaggcttttgcagcctccttgcggcagctggaagctggccactcaggccggttagagaaaacggaagatctatggctgagggttcggaaagatcatgctccccgcctggcccgcctttctctggaaagctgttctttacaggatgtcttgcttcatcgtaaacctaaactgggacaggaactgggccggggccagtatggtgtggtatacctgtgtgacaactggggaggacacttcccttgtgccctcaaatcagttgtccctccagatgagaagcactggaatgatctggctttggaatttcactatatgaggtctctgccgaagcatgagcgattggtggatctccatggttcagtcattgactacaactatggtggtggctccagcattgctgtgctcctcattatggagcggctacaccgggatctctacacagggctgaaggctgggctgaccctggagacacgtttgcagatagcactagatgtggtggagggaatccgcttcctgcacagccagggacttgtccatcgtgatatcaaactgaaaaatgtgctgctggataagcagaaccgtgccaagatcactgacttaggattctgcaagccagaggccatgatgtcaggcagcattgtggggacaccaatccatatggcccctgaacttttcacagggaagtacgataattccgtggatgtctacgcttttggaattcttttctggtatatctgctcaggctctgtcaagctccctgaggcatttgagaggtgtgctagcaaagaccatctctggaacaatgtgcggaggggggctcgcccagaacgtcttcctgtgtttgatgaggagtgctggcagttgatggaagcctgttgggatggcgaccccttgaagaggcctctcttgggcattgtccagcccatgctccagggcatcatgaatcggctctgcaagtccaattctgagcagccaaacagaggactagatgattctacttgaaagcaaagacctttctctttcactctctagttatttccttccccctcaccttttggccatggggagaatttgacatttattcactataggacacactcccaagggaactggtgcttgctgggaaacttggaaccttcccaggcagggatgactcctggacagtgaagagttgaatgactgagcatattcagcagctcactgaagcgccaagctatccctttagcaaaaaagtgtctcagatgtgtaaaagctgaggaatgtggtgttctggcttcacaaatgaaaaggaggcagatgttaccattgtcttttcactgtatatacttctaagacagcaagcgggacactgcagtggcaatagtgttaaaaaatctcattctcatgatttttggctctagctaggaatagtttagtcaggactcagaaaatttgagcttagttctgggattatgaaaacatggggacaaaaacaataacttgtggatgtctggttcctgctgtctgcagccaggtatctcaggttgcaatggtcagaagtcccagtgagggagctagaaacagagctatctgttcctcatagtgccagtgtgtttacatttataggaccacatatccctggtttctgggggagggtttcactgttacatcaccaagaggccctgaaaagaggaaaaaaaaaaaaaaagaggccaggcatggtggctcacgcctgtaatctcagcactttgggaggctgaggcgggcggatcacaaggtccagagttcaagagcagcctggccaacatggtgaaaccccgtctctactaaagatacaaaaaattagccgggcgtggtggtgcatgcctgtaatccaagatactcaggaggctgaggcaggagaatcgcttgaacctgggaggcggaggttgcagtgagccgagatcatgccattgcactccagcctgggcgacagagtgagactctgtctc

SEQ ID NO: 8 - protein, 390 aa, Homo sapiens
MLWEQIKQIIQRITWVSPPAITLEWKRKVAQEAIESLSASKLAKSICSQFRTRLNSSHEAFAASLRQLEAGHSGRLEKTEDLWLRVRKDHAPRLARLSLESCSLQDVLLHRKPKLGQELGRGQYGVVYLCDNWGGHFPCALKSVVPPDEKHWNDLALEFHYMRSLPKHERLVDLHGSVIDYNYGGGSSIAVLLIMERLHRDLYTGLKAGLTLETRLQIALDVVEGIRFLHSQGLVHRDIKLKNVLLDKQNRAKITDLGFCKPEAMMSGSIVGTPIHMAPELFTGKYDNSVDVYAFGILFWYICSGSVKLPEAFERCASKDHLWNNVRRGARPERLPVFDEECWQLMEACWDGDPLKRPLLGIVQPMLQGIMNRLCKSNSEQPNRGLDDST

SEQ ID NO: 9 - DNA, 5766 bp, Homo sapiens
cgccgccgcagccgccgccaccaacgccaacaacaacgtgtcgggagcagccgcggccgaagccggagcaggggccggagtcgcggctggagtggggcctgcgcgtgtgagtggccgagagttgcccgtgccggagacctcaaactccgggcgggagcgctttcggtgggccgagccgccgtcgcccgcggcaccgtgtccccaaggcccagacagcggggcgggggtctatcaggcgcgggactggcttctctcaggccgacccttctagccgggctccggatcttcctcggcctcgggcccccttcacgttcaggacggaggccggagaacggtgatcatcacacaccccagccggcttcctggccagggcattttctctgcggccaagcagtgttgggattgtagctgctgggagccccctcctgccgccacctctcctgcaaggaagggactgacgcgttcccactttcttactctgcacctccccactctcttcccattcaagaacgagtttctcttccctctccattggaaatcctgctccggaggtccaaaacacgtattttaagcgtgcccttcctcacctcctggagcacccttagaagtattgcctacttatccgggctgagaatccttcatttttgacctggcttttttttcgccctttgggagataaaggtccctctccaccctctactaacactctgcacccaaggccttatcctttggggtcaccagctccttggccatttctatgtgatttcccccacccatctgagttccagtttcctctgggctccaatctccagtccctggcggatctggtcagtcccacccctaggtgggagtgaccagggggctctggcccaggattcccaaccctggaaggcagctccaaggcaggaagagaattgggcatcgggtacgaacctggcagctcaggagtcggggctccactcaccccacacaaaaagatgaaaaagcgcaaagagctcaatgcattgattggtttggctggggacagccggagaaagaagcccaagaaaggcccaagcagtcaccgcctgcttcgcactgagcctcccgactcagactctgagtccagctccgaagaggaagaggaattcggtgtggttggaaatcgctctcgctttgccaagggagactatttacgatgctgcaagatctgttatccgctctgtggttttgtcatccttgctgcctgtgttgtggcctgtgttggcttggtgtggatgcaggttgctctcaaggaggatctggatgccctcaaggaaaaatttcgaacaatggaatctaatcagaaaagctcattccaagaaatccccaaacttaatgaagaactactcagcaagcaaaaacaacttgagaagattgaatctggagagatgggtttgaacaaagtctggataaacatcacagaaatgaataagcagatttctctgttgacttctgcagtgaaccacctcaaagccaatgttaagtcagctgcagacttgattagcctgcctaccactgtagagggacttcagaagagtgtagcttccattggcaatactttaaacagcgtccatcttgctgtggaagcactacagaaaactgtggatgaacacaagaaaacgatggaattactgcagagtgatatgaatcagcacttcttgaaggagactcctggaagcaaccagatcattccgtcaccttcagccacatcagaacttgacaataaaacccacagtgagaatttgaaacaggatatcctgtaccttcacaactctttagaggaggtaaacagtgccctagtggggtaccagagacagaatgatcttaaactcgagggaatgaacgagacagtcagtaatcttacccagagagtcaacctgatagaaagcgatgtggttgctatgagcaaggtagaaaagaaagcaaacctgtccttcagcatgatgggtgatagatctgccactctgaaaagacagtctttggatcaagtcaccaacagaacagatacagtaaaaatccaaagcataaagaaagaagatagttcaaattctcaggtatccaagctaagagagaaactccagctgatcagtgctcttacaaacaaacctgagagcaacaggcctccagagaccgccgatgaagagcaagtagagagtttcacatcaaagccatcagcattgccaaaattttcacagtttcttggagacccagttgagaaagctgcccaactaagacctatctccctaccaggagtttctagcactgaagatcttcaggatttattccgcaagactggccaggacgtggatgggaagctgacctaccaggaaatctggacctccctaggttctgctatgccagaaccagagagcttgagagcatttgattccgatggagatggaagatactcattcctggagctaagggtagctttaggtatctagcttcatcaggcatattttagaaatggactgcctaatatctatttacctaacaacaaaacaacccttacttacccatcagtcctctagtcctccaaactactgtagcagatactttgccaccttttaacttgtttgaagaagctatataaaagttatttttttaaagaagaagaccattttacttatgatgttcagaaatctatgatttcctacaaccagtaagatcttacattttaaaattgccagaaaaaaaattaaagccctctttttttctctttcctttttttgaggggaggagaccttatcttttaaagctgggaaatgtatatagagagagaataagccacttttatatttcacttaaatttgccttaaattagctgcactttatagagactcagaaaatgtcttttctttaaaagataggccttttctgtttgtaaatatttaaatgaaagaaagcattgtgcatattgtgtggaaagtaggaagaatggttttgaacaggatatgaacaaatgacttattaaaaattgctgatctggtgtaggtggcagctgaaactacatccatgtctccataaggcatccctcaaaggcccaggcgctgccagggggtttgtcctggtagctggaggaaccgatttcagggaatagacactggagacaatactgactccaggcatggctcatggaagtaggattctggttctttgttcctattccctcagctaatcccaacctgggaatcagagaagtcttggggatttttctcatttttagtactatttcagggtttatgagcataaaaagttatccattggggagctccattttccctgctgagtgagctagattgccttccccacccacccacttaagtctgtcttaaagccgtagctggctcccaccaccagtaccatctccatttgaatggcagggctaaattcccccagccattatctcacactgaccacccagagctttagaagagagctgtgcttctaattttgacccagaaaaccataccccttgagattttacctagaggctaaccaagagcctaatatgtttctctgggggatgactaaagccaaaaaggctgtgagatgaaacatgtgaaataatattcagtttccttaccattaccagctcagaagtagctagaggctttctacccaaaggatgccaaagtatagcagggcaggcctggagctagggccttcacatggtggtagcaagtttttcaaatctaatacaatcaagtacaatacttcctttaaatgcttctgtggacctggcatgaaagatccctagattgaaaggaataatacctccatgtctcctgtatgttgagtctagaattgctgtgttgttcttagaagcagtctttgggcaacaacttgaaaggggaaaaaaaaactacaaaaacttaactttggtataggccaagtcagggagaaagtagagaaagctgtcatgccacagacttctttagtggagatcatttcctttttaactttgttcaggttgcccttcaccatggatacagtccggtacccttaaacatttaagggctgtttttttttttctttatatgatgttcagcttggtattaaccaaacttaaattttttttccagaagtattaaaatttagttaaagcaagatgaagtcttttcccatgaatgtgtccccttatctcattatagcttatgctcagtttcactttcttggaaaggttaaaacaaattagcctggcactttaggtaacttgaaaataactctcacccttctgactgccttccatccactctcaccccacacctttttttaatatatatacacccttacagattttctaacaaccaaataaaattctagcaataaattgaattatactgctatatttgtatatactgtaccataaatagtatgtctgtacctggaaggtatttttttgagaactgatttatatgaaatatacttgagggtaatgtagcttgtcctttttagtttcaaattcttattcataggcagatactttgaagacaccttaactctttgttgtgtgtattttcccttgcatcagataactatacatggagaaatccattttgttttttggtttatcttctgttcttcctaccttgtgcttgtctgactagtatcacccctgagccagtatacagtaccatcctctccccctaccaagtttggcagaggtgtttatactgcattctcagatctatgaatatttttgtcatcctgcctgagaaagtacatcttccaggcaaaagtagggtatcttgaactcctttcttctgagttgagcagactcactggcttaagcatcccataagcctattatttagtgtgaatttgggacttttatttgtatgttgtcttaacttactgagcctgtgacttgtatttagctgtagtcaagcctcaccagccatatctcatctctgagaggaagactggatagtgagtttggttttggttttggttttaaacccttttaccttttcagtctggcagctcctactgctgctttcctaagttactcaaagcactgcttcttggtccttgttttcttttgccttgtactatcttgtataatgtttactaaaagagcctccccataaatcagagaggcaaaaaggtagcatataaaaagaagaaccaaaagaagagaaaacctgacttctagatgactgtggtgtcatctaaaaggcagaggtgcctgaccctcaggaatgtgtatgatgcaacagagctgaagaatgggttcccttcacctactgctggccattgtcccttctgttggagattcctttctttatggttgctgttcataaccccttcaacacccccccttgctggtcctgccggtcacagttgaggattttggctgtgatgggctcatactcataatggcctgttgagttgtttttagtgacaggttttgtttgctgcacacctcattggcttgcttaccaacaccagctagaagtggtccctctttcatatccaaaccagaacacatttgggtattcctgagactttatagagcgtgtattgtttgttatacgaacctaacctaccttgttgttttctcatattaagaaatttaaatctactttgttttagtgaagctattttggtaatgtataagcaattttggttctgtttagggagaattcagggtttccatgacttgatttgagctttgcatatttcaaataagtaacacagccattttaatttagctaaataaaaaaatttcttttttctctc

SEQ ID NO: 10 - protein, 495 aa, Homo sapiens
MKKRKELNALIGLAGDSRRKKPKKGPSSHRLLRTEPPDSDSESSSEEEEEFGVVGNRSRFAKGDYLRCCKICYPLCGFVILAACVVACVGLVWMQVALKEDLDALKEKFRTMESNQKSSFQEIPKLNEELLSKQKQLEKIESGEMGLNKVWINITEMNKQISLLTSAVNHLKANVKSAADLISLPTTVEGLQKSVASIGNTLNSVHLAVEALQKTVDEHKKTMELLQSDMNQHFLKETPGSNQIIPSPSATSELDNKTHSENLKQDILYLHNSLEEVNSALVGYQRQNDLKLEGMNETVSNLTQRVNLIESDVVAMSKVEKKANLSFSMMGDRSATLKRQSLDQVTNRTDTVKIQSIKKEDSSNSQVSKLREKLQLISALTNKPESNRPPETADEEQVESFTSKPSALPKFSQFLGDPVEKAAQLRPISLPGVSSTEDLQDLFRKTGQDVDGKLTYQEIWTSLGSAMPEPESLRAFDSDGDGRYSFLELRVALGI

SEQ ID NO: 11 - DNA, 6550 bp, Homo sapiens
tcccggaaggcaaatggctggcgtggaagcacaacccgctttcactcttcgaatttgtgcttagctcttttcttgtaccttgcgactcgtgaccaacatgctgtgatgtgtgccgagggaggaattggtcagctacacaacctggatcttaccacagtttggatatgactgaggctctccaatgggccagatatcactggcgacggctgatcagaggtgcaaccagggatgatgattcagggccatacaactattcctcgttgctcgcctgtgggcgcaagtcctctcagacccctaaactgtcaggaaggcaccggattgttgttccccacatccagcccttcaaggatgagtatgagaagttctccggagcctatgtgaacaatcgaatacgaacaacaaagtacacacttctgaattttgtgccaagaaatttatttgaacaatttcacagagctgccaatttatatttcctgttcctagttgtcctgaactgggtacctttggtagaagccttccaaaaggaaatcaccatgttgcctctggtggtggtccttacaattatcgcaattaaagatggcctggaaggttatcggaaatacaaaattgacaaacagatcaataatttaataactaaagtttatagtaggaaagagaaaaaatacattgaccgatgctggaaagacgttactgttggggactttattcgcctctcctgcaacgaggtcatccctgcagacatggtactactcttttccactgatccagatggaatctgtcacattgagacttctggtcttgatggagagagcaatttaaaacagaggcaggtggttcggggatatgcagaacaggactctgaagttgatcctgagaagttttccagtaggatagaatgtgaaagcccaaacaatgacctcagcagattccgaggcttcctagaacattccaacaaagaacgcgtgggtctcagtaaagaaaatttgttgcttagaggatgcaccattagaaacacagaggctgttgtgggcattgtggtttatgcaggccatgaaaccaaagcaatgctgaacaacagtgggccacggtataagcgcagcaaattagaaagaagagcaaacacagatgtcctctggtgtgtcatgcttctggtcataatgtgcttaactggcgcagtaggtcatggaatctggctgagcaggtatgaaaagatgcattttttcaatgttcccgagcctgatggacatatcatatcaccactgttggcaggattttatatgttttggaccatgatcattttgttacaggtcttgattcctatttctctctatgtttccatcgaaattgtgaagcttggacaaatatatttcattcaaagtgatgtggatttctacaatgaaaaaatggattctattgttcagtgccgagccctgaacatcgccgaggatctgggacagattcagtacctcttttccgataagacaggaaccctcactgagaataagatggtttttcgaagatgtagtgtggcaggatttgattactgccatgaagaaaatgccaggaggttggagtcctatcaggaagctgtctctgaagatgaagattttatagacacagtcagtggttccctcagcaatatggcaaaaccgagagcccccagctgcaggacagttcataatgggcctttgggaaataagccctcaaatcatcttgctgggagctcttttactctaggaagtggagaaggagccagtgaagtgcctcattccagacaggctgctttcagtagccccattgaaacagacgtggtaccagacaccaggcttttagacaaatttagtcagattacacctcggctctttatgccactagatgagaccatccaaaatccaccaatggaaactttgtacattatcgactttttcattgcattggcaatttgcaacacagtagtggtttctgctcctaaccaaccccgacaaaagatcagacacccttcactgggggggttgcccattaagtctttggaagagattaaaagtcttttccagagatggtctgtccgaagatcaagttctccatcgcttaacagtgggaaagagccatcttctggagttccaaacgcctttgtgagcagactccctctctttagtcgaatgaaaccagcttcacctgtggaggaagaggtctcccaggtgtgtgagagcccccagtgctccagtagctcagcttgctgcacagaaacagagaaacaacacggtgatgcaggcctcctgaatggcaaggcagagtccctccctggacagccattggcctgcaacctgtgttatgaggccgagagcccagacgaagcggccttagtgtatgccgccagggcttaccaatgcactttacggtctcggacaccagagcaggtcatggtggactttgctgctttgggaccattaacatttcaactcctacacatcctgccctttgactcagtaagaaaaagaatgtctgttgtggtccgacaccctctttccaatcaagttgtggtgtatacgaaaggcgctgattctgtgatcatggagttactgtcggtggcttccccagatggagcaagtctggagaaacaacagatgatagtaagggagaaaacccagaagcacttggatgactatgccaaacaaggccttcgtactttatgtatagcaaagaaggtcatgagtgacactgaatatgcagagtggctgaggaatcattttttagctgaaaccagcattgacaacagggaagaattactacttgaatctgccatgaggttggagaacaaacttacattacttggtgctactggcattgaagaccgtctgcaggagggagtccctgaatctatagaagctcttcacaaagcgggcatcaagatctggatgctgacaggggacaagcaggagacagctgtcaacatagcttatgcatgcaaactactggagccagatgacaagctttttatcctcaatacccaaagtaaagatgcctgtgggatgctgatgagcacaattttgaaagaacttcagaagaaaactcaagccctgccagagcaagtgtcattaagtgaagatttacttcagcctcctgtcccccgggactcagggttacgagctggactcattatcactgggaggaccctggagtttgccctgcaagaaagtctgcaaaagcagttcctggaactgacatcttggtgtcaagctgtggtctgctgccgagccacaccgctgcagaaaagtgaagtggtgaaattggtccgcagccatctccaggtgatgacccttgctattggtgatggtgccaatgatgttagcatgatacaagtggcagacattgggataggggtctcaggtcaagaaggcatgcaggctgtgatggccagtgactttgccgtttatcagttcaaacatctcagcaagctccttcttgtccatggacactggtgttatacacggctttccaacatgattctctattttttctataagaatgtggcctatgtgaacctccttttctggtaccagttcttttgtggattttcagggacatccatgactgattactgggttttgatcttcttcaacctcctcttcacatctgcccctcctgtcatttatggtgttttggagaaagatgtgtctgcagagaccctcatgcaactgcctgaactttacagaagtggtcagaaatcagaggcatacttaccccataccttctggatcaccttattggatgctttttatcaaagcctggtctgcttctttgtgccttattttacctaccagggctcagatactgacatctttgcatttggaaaccccctgaacacagccgctctgttcatcgttctcctccatctggtcattgaaagcaagagtttgacttggattcacttgctggtcatcattggtagcatcttgtcttattttttatttgccatagtttttggagccatgtgtgtaacttgcaacccaccatccaacccttactggattatgcaggagcacatgctggatccagtattctacttagtttgtatcctcacgacgtccattgctcttctgcccaggtttgtatacagagttcttcagggatccctgtttccatctccaattctgagagctaagcactttgacagactaactccagaggagaggactaaagctctcaagaagtggagaggggctggaaagatgaatcaagtgacatcaaagtatgctaaccaatcagctggcaagtcaggaagaagacccatgcctggcccttctgctgtatttgcaatgaagtcagcaagttcctgtgctattgagcaaggaaacttatctctgtgtgagactgctttagatcaaggctactctgaaactaaggcctttgagatggctggaccctccaaaggtaaagaaagctagataccctccttggagttgcaagtattctttcaaggttggaagagggattttgaagaggtatctctccaagcaagaatgacttgtttttccataagggacatgagcattttactaggcttggaagagctgacatgatgagcattattgtatgtttgtatatacatttgtgatagagggctagagtttgacctagagagagtttaaggaagtgaaatatttaattcagaaccaaatgcttttgtaaaactttttggattttgtaaaagcattttcattctcttagaaattcaagtattttcaaggggagtcatttgagatatatttattttactaggagatcttatattctagggaaatgctttaaatggtcaggctccagtcggaatttttttaagaaaaaagtagtttttaatacattggttaggactcagaggaaatacggaaaaaacattgtagatggttaatttacagataaaatcccaagagccttttaaacaacaaggtacctaaaatagggtataattatactgcttaaaatacagttagtgcctattaatagctttttatttcctatgggaagatgcttttggtcttctggctgaagatgtaggcatacctctcactcatttcaatgttttcctgaggtggagccttcattggaaaggggaaagagggattctagggtttcatcagggaccaggaatgcattcctctgtcaggttccaatcaagagaagaccttttatgagatctgcctctgtatagatgttgtcagttaggaaactgaagccataggtcaggcagacatcagctcagcctgtggcccattgggtgatttcctgtattttaaaactgacagtagcctgatcaaagtgatacaatcaatttcaaaacaatcttccagagaccacttgaaggttcatagtttttacaataccctgagacttttcaggtgttggagcctctaaaatatgagatataaacagaaactaatacaagttgttctctggaggtttctatgaggttcttagaaaaatttggttttaaaatcatttgaggacaggaatgtctatagcaagtttactcctattgcgaatcatgtatgctggctttagttgtaacaaacgattttattctaagtaaggccaggtgctactataaaatcatatattcctgttgaagttcttttgaagtcctatctctatttattatatttgaaagttgtcagccaccagtcatccagaatttccttcctgaatctccatgctcatatgcaatgtctacatcaaggtcttcttaatgactattattctcagggtttagttttctaccttctgcctactattttggtctgacatttttgtagccttctgttattattggaaatagtctcttacataagctgatttcgagaactttcaaaatctcacatagctaatggaagttgctttctgctttcttatgactgtttttataaataaactgtttcataaataatgtatactggaaaattgtcatcactcttgtaaacatttgccaaatccaactgtaattagtaccagaacttagaaggaaagtcacaaagcatgccttttaccagaatattgctttgccttttcttatatggaaaatcactgatgtcatatcatttattatcctcacagcagctggcattcattccctgaaatcgtcgtctcctgataaacaataatttagagatatgttcacttgtaaataaaaaggttagaatctaaggacaaagaatgatccaaaaatactaaggaaaaagaacaattttcaattttcagaaatgaattatctgtacatatgaatagaggaaaatactgagtgctaatgttacagggccacaatctcatatccacaattccaaaatctccaaacctctgaaaactgaaatatttttcataaattaggcgtaaattctatgccctgaacaaatgtaaagctatttatgtactttgcttaatgtaatcattcatatactttgctacaaaaatattaatatatttcattactgaatgctaaactgtatttctttttaaaatttggaaaatttttaattctgaaacacaatctagaccttag

SEQ ID NO: 12 - protein, 1426 aa, Homo sapiens
MTEALQWARYHWRRLIRGATRDDDSGPYNYSSLLACGRKSSQTPKLSGRHRIVVPHIQPFKDEYEKFSGAYVNNRIRTTKYTLLNFVPRNLFEQFHRAANLYFLFLVVLNWVPLVEAFQKEITMLPLVVVLTIIAIKDGLEGYRKYKIDKQINNLITKVYSRKEKKYIDRCWKDVTVGDFIRLSCNEVIPADMVLLFSTDPDGICHIETSGLDGESNLKQRQVVRGYAEQDSEVDPEKFSSRIECESPNNDLSRFRGFLEHSNKERVGLSKENLLLRGCTIRNTEAVVGIVVYAGHETKAMLNNSGPRYKRSKLERRANTDVLWCVMLLVIMCLTGAVGHGIWLSRYEKMHFFNVPEPDGHIISPLLAGFYMFWTMIILLQVLIPISLYVSIEIVKLGQIYFIQSDVDFYNEKMDSIVQCRALNIAEDLGQIQYLFSDKTGTLTENKMVFRRCSVAGFDYCHEENARRLESYQEAVSEDEDFIDTVSGSLSNMAKPRAPSCRTVHNGPLGNKPSNHLAGSSFTLGSGEGASEVPHSRQAAFSSPIETDVVPDTRLLDKFSQITPRLFMPLDETIQNPPMETLYIIDFFIALAICNTVVVSAPNQPRQKIRHPSLGGLPIKSLEEIKSLFQRWSVRRSSSPSLNSGKEPSSGVPNAFVSRLPLFSRMKPASPVEEEVSQVCESPQCSSSSACCTETEKQHGDAGLLNGKAESLPGQPLACNLCYEAESPDEAALVYAARAYQCTLRSRTPEQVMVDFAALGPLTFQLLHILPFDSVRKRMSVVVRHPLSNQVVVYTKGADSVIMELLSVASPDGASLEKQQMIVREKTQKHLDDYAKQGLRTLCIAKKVMSDTEYAEWLRNHFLAETSIDNREELLLESAMRLENKLTLLGATGIEDRLQEGVPESIEALHKAGIKIWMLTGDKQETAVNIAYACKLLEPDDKLFILNTQSKDACGMLMSTILKELQKKTQALPEQVSLSEDLLQPPVPRDSGLRAGLIITGRTLEFALQESLQKQFLELTSWCQAVVCCRATPLQKSEVVKLVRSHLQVMTLAIGDGANDVSMIQVADIGIGVSGQEGMQAVMASDFAVYQFKHLSKLLLVHGHWCYTRLSNMILYFFYKNVAYVNLLFWYQFFCGFSGTSMTDYWVLIFFNLLFTSAPPVIYGVLEKDVSAETLMQLPELYRSGQKSEAYLPHTFWITLLDAFYQSLVCFFVPYFTYQGSDTDIFAFGNPLNTAALFIVLLHLVIESKSLTWIHLLVIIGSILSYFLFAIVFGAMCVTCNPPSNPYWIMQEHMLDPVFYLVCILTTSIALLPRFVYRVLQGSLFPSPILRAKHFDRLTPEERTKALKKWRGAGKMNQVTSKYANQSAGKSGRRPMPGPSAVFAMKSASSCAIEQGNLSLCETALDQGYSETKAFEMAGPSKGKES

SEQ ID NO: 13 - DNA, 8083 bp, Homo sapiens
ctcgggcagcccagtctttgccatccttgcccagccggtgtggtgcttgtgtgtcacagccttgtagccgggagtcgctgccgagtgggcgctcagttttcgggtcgtcatggctggctacgaatacgtgagcccggagcagctggctggctttgataagtacaagtacagtgctgtggataccaatccactttctctgtatgtcatgcatccattctggaacactatagtaaaggtatttcctacttggctggcgcccaatctgataactttttctggctttctgctggtcgtattcaattttctgctaatggcatactttgatcctgacttttatgcctcagcaccaggtcacaagcacgtgcctgactgggtttggattgtagtgggcatcctcaacttcgtagcctacactctagatggtgtggacggaaagcaagctcgcagaaccaattctagcactcccttaggggagctttttgatcatggcctggatagttggtcatgtgtttactttgttgtgactgtttattccatctttggaagaggatcaactggtgtcagtgtttttgttctttatctcctgctatgggtagttttgttttctttcatcctgtcccactgggaaaagtataacacagggattcttttcctgccatggggatatgacattagccaggtgactatttcttttgtctacatagtgactgcagttgtgggagttgaggcctggtatgaacctttcctgtttaatttcttatatagagacctattcactgcaatgattattggttgtgcattatgtgtgactcttccaatgagtttattaaactttttcagaagctataaaaataacaccttgaaactcaattcagtctatgaagctatggttcccttattttctccatgcttgctgttcattttgtctacagcgtggatcctttggtcaccttcagatattttagagctacatcctagagtattctactttatggttggaacagcttttgccaacagtacatgtcagctgattgtttgccaaatgagtagtacccggtgtccaactttgaattggttgctggttcctctcttcttggttgtcttagtggtaaacctaggagtagcctcttacgttgagagcattctcctgtatacattaacaactgcttttactctggcccacatccattatggagtacgagtggtaaagcagctgagcagccattttcagatttaccccttctcattgaggaaaccaaactcagattgactaggaatggaagaaaagaatattggcctgtaataatctttctttgggcacaaagaagtactgtaaataaatgcttgtaaatatttcctccatcaccattgaactagactgatctgcttgacagacgtgggatctcagtatggtacttggacagcaggaatgatacatataatctgaacttgggaaattttggacctactaactctaagcctattttattttttataaaactatgtgacattttggttgagcagaatgtacgttagaccagcaaaatgttcctaatggttctaacttcgtgagtttacaatgttgtgattcatgcagggttaaagatgctttgtttttattttttaagtaccaaaattggtttcagaacactgataacactcagaaaaccacagtgtgttttcatatttggaactttgtaatagcgggagtagcagtagtccaaacctagtatagggaaaggataaaaataagtcaccttcaccaagagatgccaatgattaccaaacaacagacagttgccaaatactggtttctctttcccctgaaaatggcttttgttctcaaatgataagagagctaatacatttagctaatattctagctctctttattatggaacagatcttgatagatggtttaattttctcctaaagagaaataatcagttgagaatttgagaatgggttgtaattatcgctcacccattgggatggttcattgtttaaatatggcattttccccccttcagctgcaggttcctgagatttggtgcctgtgagctctgattgtaggaatgcatgtgacagtcccagtcctatggtaatgacttaggaggaatgcagataaaagtaccttgtaagataaatataaattggagttaggaatttcatgaacctcactatgaccaaaattaattttttgattcagtttgtctgtctgtctgtccttcccctctcttcttttttcagggtgaggtgctgtgtttcttatttcatacgagataaaacagagagaagttctctcttctccagcttgtccatttccccacttgaagaaaacttttgatatatatgccttactgagtacatgccccctttaatgttaacatgacttggagtaatttctgaggtttactgacaaacataaaaatccctttaattgtagtgtagttgttctataaaccatattttttcatgatgtggatattttcttctatttctttgtcttcatttaatttggtggtggtgaactttacttgctgattttcttttatttttcactgaatgaagtttgtgcttgaatgaagagtgtatcttaaaccccctttttttggacaggctgcacttggataaaataggcaccactgtgttgatatgtaattaaattcataactatttatgaatgtgaccattgttaaatttaattatagtgtatttctttcttgaatttcagtctgctgctttgtaaaaaataactgtaattgatatttcatttttaaaatttataaattccaatcattgtcattcaagttattagaaatgagtcacggcactaactcatggttctatactcattttaagaaattgtaaaggttgaggtgtatttggtgactttctctgagacacagatgaacgttggaaatagatgagaaatggatgcagaataatgcagatagtgatcccatgtttgttaattgccatcatggaagtcagagaattaaccaattaagtgacggtaactattttttgtataccttgttctttgtgatctataaaattaaactggataaaatgttaattgggggtagggaaaaagctttgttttaagatttctaagctaattattttaccgttttgttgaccttgtacacaagaatgagattaatctgtattatataagaaaagaactgataaatatttataaaatgacaaacttggaccacaggctttcttgaccttgtacagtatagtaaaaataccctttacttctgacattttaaggtcacgtcctatagtttgggggcccttttacttcaaaggtgtgtttctgccctcttttggttgtatcaaatttatttgaaaattgttttgtgctgtactttatgacttgtttccctatggtggtaaagaatgatgccatagtcagttttattgacattgttagatgaaagaaaagtagctgtcaacaaggggaaagtgaaattgagcaattcactgtttcaggatacttgaggatctttagtgtactgctgactctttttcttgttttgtctgccattcttgaaagtgcatcttgagagcccccatttgttgtgtagagatatagcagctgaagcaggacttcattccagatagctgtgtgcatttatgtcatgccaattactataacatttccaaagtatgaaaggtctttcaggcttatgcatgatattgtgggtgacttcatgccccagcctttctccccagcatacagtgagctgccttttggggagcaatgccatgataaggagcatacgtgcatgttttccgtactttcagaatagagtgccttagctaggccagaggctcgtattgtgtttctgctgaccaaagttgcatacacctgtaagaagtgtctttttaggttatggtattaaaaaagtcgtttcaggcttatcagataataattctcctatatttgaggggggcatgggataaaaagatttaataatacctggatagagaatgctaatgctgccttttgtgatcctttaatattatccatgcttttataaagtgcagtacacattttgacattgccacagtttacagtgtcattcttccacgaataggtaattgatagagtttatgggtcttagtcttaagggtaacagagtgaataccttccaaataaaaagctaaatattcctccttgtcattttaatgtcattattgaaatacttttaattctaattttcaaaagaaagctaaatgctgattgtggtttaggatgttacagtactaataccatcatgttatttattctaatatagaggcattgagaggaggggagtgtatgtctagagttaccccaattctaaataggtaccaaaagagtattgattatgacctggagcctgagctttgggagaatatacaaaggaaaaagcaggcatagtttccactttaaagggaagaagggactttaacatcctatgaatttgaaacatcctatgaatttgaaagtttcctaagtgttgttttcaaagaactggtttattttatagtcagtcaaaatgcatttggaatgtaccttgcttccataacagactttgactctatgacctatgaagaataaaaagataatctgaaggtgtttgagtcagaggctacttttcccctttatattttatatagaagcctatttgtaattcttcctcttcgttcttcacctgggtttcctcttatctacccaccacctaggacagaggctgcccactcaggtcttgccttgttgcccctagatgttgccacattcacatctccttgatggccttagatctcttgactagtcaaggactgggaacgtgccagggggaacaaagtaagcttgatgggctggaacttttgactttgggtccccaaacagccaaaaacagatacagaaatctttggttgtgtggatgcctgtgttcagagtggccttatcttgacctcagttctggatttaagtactctatagaaaatttgtgaagtcagaccagaattttctttgtcccatgaggtgtctttgtaagtcagcatgctcttgcttgaacaccctgtgggtgaataactttagtaagttagctgccagcattagaaccttccaggaagaattttaactagtatgctacatttgaagcactctgacatgtacatttaaagtgacagttttaaaagtaaaatggtgttaaatctctgaaacttgaacgtatgatgctctggacattttaggaagcttaagtaaatttctgattttcctttattgattatgtaacccacgtgatttgcatattttagtttttatgtttgacagtggtacaaatgactgacattggcaggcagttattgtagggtatatggttaatcacgattgtctaacttttccagagaaagtggtgatatgcttcttccttgcatggagctgtgggaattggatttgttggctcttcatttccagtgtttctacagtgcagaataaatgtaactttttaagcctgcatgctagtaatatgctttgctctttagtaaacaaatatgcaatagatagtaatctatctactttttttaaagcagggatctgctccctatctagacagatcttttaatgcattaatatttagagtagcaaaagaagttttgccatttgtagctctctcacaggtagcttaagcgagggggttgtgttgcatgtgcttagaaaggccaggatcccagaattggaaggtgccacatatacttttagaatattttaacaaaggcttgtctctaggaaaatccacttggactgcagcaggattgttctggtcttccacagaccattcacatggtctttctctccagagaatcattcttagaaagccaggctaaatcagagtagtgctgtttttaaagcaagtatattggatgatggctatagggtaccttaatgctgtgttgaaattggaagcagactgctcttcagcagcacatatactgcaactgaagttagagacagtactgaagacgagggagtagctaaatgctgtggatatttcatgtctgtttggctaatgtcttctcttgaggagtttctatgggtagcaattaaaattagaatttattttaaggtttttattaaaatatatacaatttgccataaattctagattctgatattcttgacaagcactttttgaatatctcaagagtaagttcttgacctggaacatatatagttagtacttgatgtcactaaccaagttgtagaaggagcctgttataattctgcaagatctgtgaatagcattataaatctggggcaggtgcctgtaatcccagcactttgggaagccaaggcaggtggattgcttgagcccaggagttcgacaccaacctgagcaacatggcgaaaccccgtctgtaataaaaatacaaaaaaattagctgggcatggtggcatgtgcctgtaatcccagttactggggaggctgaggcaggagaatcacttgaacctgggagatggagattgcagtgagcagagattgtgccactgcactccagccagagtgacagagcaagactccatctcaaaaaataataataaaaataaacaaatctgtttaaattactgttgcctttcagcattagactttgagtttaataactacaaattgagactgctcacaatattaactttttttgtaggttattttgttgtttagataacttgcctccctagaggagcattcaggagataaagacctagctacatgtaatgatatgatcatttcaaaaatgtgccaagaaagcaaaattcattattgaactctaaatttctgggtgtttttttttttaaagtagcattttctctgggtaaagggaaaggcaatgaatgattgccaacatgtaaactccctgctgcccgccttcccccagtcccctccatctaacataatacagtaaatttgtagccagttgtagaaaaagaaattgatatctttctgagtaaggtttcatgccctgtgactaagaataggtggaggaatcatggccaaatcaaatatgaattgtttagcttggtcctgttgcagttggctttctgtagtgttctgaaacaagaggacgattccattccttctcaggaacctagaaacaacaccgcttgagcaactggaatatgtttgctgcaagcagaatattttggggagaggaagagtagtttaattcaagtagtttaattgaacatattagtcattggtctgtctgggacgtgcagtgttcatagtagcaatgtatgtaccatttattttatctggttgtgtgtgatgtgtgtgtatgcctatttaatatgtacacatatattcactacatatgtatgtatgatatattcatatatacatgcagtctgcttgattatcagcaaaatggtcagcctttatcagatagtttcttcatgtggagttcatctgcatgtggcccttactctgaagcctcttcctgatctggagccacagtctgtctgtcttccagttcatctcagtcctcgagaaaggccctttaaatatgtcactttcccattttcctttaaccatgggttgtgtgagccagaaagagctttgagaaagatggctgcttccaccagggtggaggcttctaggtctgcatgatgatggggcccgtttctggccagagggtggctctgggagcagttgtgctgcgggcttgctgggggagaactctaactgttgcagaaacagagcttcatggcttgcttaaattacttagctggaatattttaaagtgtcagataatgtgatgtacaaagagagtatgccgatgcatttcattgtttttgcattatctgtatcagtgtttcccattgctagcccgaataatcaagaccagactgtttcttgtcactggattctggaagccttgccctttcaactggatttatgtttgtttctgctgctgtgaaaccccaaagtaatgcagtaggttttaattgaaaattgtttaattctattattgataattatttattgtaataaaagtaaaagagtaatttttaaatcctttt

SEQ ID NO: 14 - protein, 386 aa, Homo sapiens
MAGYEYVSPEQLAGFDKYKYSAVDTNPLSLYVMHPFWNTIVKVFPTWLAPNLITFSGFLLVVFNFLLMAYFDPDFYASAPGHKHVPDWVWIVVGILNFVAYTLDGVDGKQARRTNSSTPLGELFDHGLDSWSCVYFVVTVYSIFGRGSTGVSVFVLYLLLWVVLFSFILSHWEKYNTGILFLPWGYDISQVTISFVYIVTAVVGVEAWYEPFLFNFLYRDLFTAMIIGCALCVTLPMSLLNFFRSYKNNTLKLNSVYEAMVPLFSPCLLFILSTAWILWSPSDILELHPRVFYFMVGTAFANSTCQLIVCQMSSTRCPTLNWLLVPLFLVVLVVNLGVASYVESILLYTLTTAFTLAHIHYGVRVVKQLSSHFQIYPFSLRKPNSD

SEQ ID NO: 15 - DNA, 2788 bp, Homo sapiens
atagaaggaagaggagcgaacatggcagcgcgttggcggttttggtgtgtctctgtgaccatggtggtggcgctgctcatcgtttgcgacgttccctcagcctctgccaaaagaaagaaggagatggtgttatctgaaaaggttagtcagctgatggaatggactaacaaaagacctgtaataagaatgaatggagacaagttccgtcgccttgtgaaagccccaccgagaaattactccgttatcgtcatgttcactgctctccaactgcatagacagtgtgtcgtttgcaagcaagctgatgaagaattccagatcctggcaaactcctggcgatactccagtgcattcaccaacaggatattttttgccatggtggattttgatgaaggctctgatgtatttcagatgctaaacatgaattcagctccaactttcatcaactttcctgcaaaagggaaacccaaacggggtgatacatatgagttacaggtgcggggtttttcagctgagcagattgcccggtggatcgccgacagaactgatgtcaatattagagtgattagacccccaaattatgctggtccccttatgttgggattgcttttggctgttattggtggacttgtgtatcttcgaagaagtaatatggaatttctctttaataaaactggatgggcttttgcagctttgtgttttgtgcttgctatgacatctggtcaaatgtggaaccatataagaggaccaccatatgcccataagaatccccacacgggacatgtgaattatatccatggaagcagtcaagcccagtttgtagctgaaacacacattgttcttctgtttaatggtggagttaccttaggaatggtgcttttatgtgaagctgctacctctgacatggatattggaaagcgaaagataatgtgtgtggctggtattggacttgttgtattattcttcagttggatgctctctatttttagatctaaatatcatggctacccatacagctttctgatgagttaaaaaggtcccagagatatatagacactggagtactggaaattgaaaaacgaaaatcgtgtgtgtttgaaaagaagaatgcaacttgtatattttgtattacctctttttttcaagtgatttaaatagttaatcatttaaccaaagaagatgtgtagtgccttaacaagcaatcctctgtcaaaatctgaggtatttgaaaataattatcctcttaaccttctcttcccagtgaactttatggaacatttaatttagtacaattaagtatattataaaaattgtaaaactactactttgttttagttagaacaaagctcaaaactactttagttaacttggtcatctgattttatattgccttatccaaagatggggaaagtaagtcctgaccaggtgttcccacatatgcctgttacagataactacattaggaattcattcttagcttcttcatctttgtgtggatgtgtatactttacgcatctttccttttgagtagagaaattatgtgtgtcatgtggtcttctgaaaatggaacaccattcttcagagcacacgtctagccctcagcaagacagttgtttctcctcctccttgcatatttcctactgaaatacagtgctgtctatgattgtttttgttttgttgtttttttgagacggtctcgctgtgtcacacaggctggagtgcagtggcgtgagctcggctgactgcaaactctgcctcccaggtttaagcgattctcctgtcacagcttcccaagtagctgggatttacaggtgtgcaccgccatgccaggctaatttttgtgtttttagtagagacagggtttcgccaagttgtccaggctggtcttgaactcctgggctcaagtgatccgcccgcctcagtctcccaaagtgcgaggatgacatgtgtgagctaccacaccagcaatgtctatgcttctcgatagctgtgaacatgaaaagacatctattgggagtccgaggcaggtggattgcttgaggccaggagttagagaccagcctggccaacaaggcaaaaccccgtctctactaaaaatatgaaaattagctgggcttggtggctcatgcctataatcctagctacttgggaggctgaggcacgagacttgcttaatacctgggaggcggagattgcagtgagccgagatcacgctactgcgctccagcctgagtgatagagtgagactctgtctcaaaaaaaagtatctctaaatacaggattataatttctgcttgagtatggtgttaactaccttgtatttagaaagatttcagattcattccatctccttagttttcttttaaggtgacccatctgtgataaaaatatagcttagtgctaaaatcagtgtaacttatacatggcctaaaatgtttctacaaattagagtttgtcacttattccatttgtacctaagagaaaaataggctcagttagaaaaggactccctggccaggcgcagtgacttacgcctgtaatctcagcactttgggaggccaaggcaggcagatcacgaggtcaggagttcgagaccatcctggccaacatggtgaaaccccgtctctactaaaaatataaaaattagctgggtgtggtggcaggagcctgtaatcccagctacacaggaggctgaggcacgagaatcacttgaactcaggagatggaggtttcagtgagccaagatcacaccactgcactccagcctggcaacagagcgagactccatctc

SEQ ID NO: 16 - protein, 335 aa, Homo sapiens
MAARWRFWCVSVTMVVALLIVCDVPSASAKRKKEMVLSEKVSQLMEWTNKRPVIRMNGDKFRRLVKAPPRNYSVIVMFTALQLHRQCVVCKQADEEFQILANSWRYSSAFTNRIFFAMVDFDEGSDVFQMLNMNSAPTFINFPAKGKPKRGDTYELQVRGFSAEQIARWIADRTDVNIRVIRPPNYAGPLMLGLLLAVIGGLVYLRRSNMEFLFNKTGWAFAALCFVLAMTSGQMWNHIRGPPYAHKNPHTGHVNYIHGSSQAQFVAETHIVLLFNGGVTLGMVLLCEAATSDMDIGKRKIMCVAGIGLVVLFFSWMLSIFRSKYHGYPYSFLMS

SEQ ID NO: 17 - DNA, 3239 bp, Homo sapiens
gcttggggaacgaggagcaggacgcggcctcggtggggcccgggccgaacggctgcggacacctgggcgccgaggagccgagcgccgccgtctccggcatggatcagtgcgtgacggtggagcgcgagctggagaaggtgctgcacaagttctcaggctacgggcagctgtgcgagcgcggcctggaggagctcatcgactacaccggcggcctcaagcacgagatcctgcagagccacggccaagatgctgaattatcagggacactttcacttgttttgacacagtgctgtaaaagaataaaggatactgttcaaaaattggcctccgaccacaaagacatccacagcagtgtttctcgggttggaaaagccattgataagaattttgattctgacattagcagtgtgggaatagatggctgctggcaggcagacagccaaaggcttctcaatgaagtgatggtggagcacttctttcgacaaggaatgctggatgtggctgaggagctctgtcaggaatctggtctttctgtagacccaagtcagaaggaaccatttgtggagttaaatagaatattagaggcattaaaggtcagagttctgagacctgctctggagtgggcagtgtcaaaccgggaaatgcttatagctcaaaacagctccttggaatttaagctacacagactgtattttattagcttgttaatgggtggaaccacaaatcagcgagaggcattacaatatgctaaaaattttcagccatttgccctaaatcatcaaaaagacattcaggttttgatgggaagccttgtgtacctgagacaagggattgagaactcaccatatgttcacctacttgatgcaaaccagtgggctgatatctgtgacatctttacacgggatgcttgtgccctcctggggctctccgtggagtcccctctcagtgtcagtttctcagcaggttgtgtggcgctgccagctttaattaacatcaaagccgtgattgaacagaggcagtgtactggagtttggaaccagaaagatgaattacctattgaagtggaccttggtaaaaagtgctggtatcactctatatttgcctgccccattcttcgtcagcaaacaacagataacaatccacccatgaaattggtctgtggtcatattatatcaagagatgccctgaataaaatgtttaatggtagcaaattaaaatgtccctactgtccaatggaacaaagtccaggagatgccaaacagatatttttctgaagagataactttagtttgcaatttgtaagtgaaactgaatcgtgggtgcatttcagaagagaacgttccatataatgcagctaaccaaggactcctgtgtttctataagctaatgctccagaaactttgccaacctgttagtgtacacacactgaggggagtgctcccggtgaatattatcatagggctttattatattcttggtcttcatttctgatcaagtaaatacaccagcagttgtcattcaatgcaggtttttgtacttaattatatggtgatttttttactttttaagagcagaaacggaaattgacctccccgccatgtgtttaatattcctcctgcttttacttttgtcattttcttgataatcgtaagccttgagagtgtttgtgaaaaagttttatttcctgttatgtatacataattaaatgaaaattcttcagaaaaagtttgataaattgaattgtggttatgaaactaatttgcatttttatttgcttaagaaagaaagctgtgatagattccagatatgctttttgatgttttcctctgctccagctccaagaagtcagcacacctgcattttagctctgcatgcagccccagcaggctgcgtgtttaagaatttcattgtttaactggctggtgtgagaagtcttccgttagcatagagtggaaggagtactattgtttggttgggtttttgtttgtttgttttttgtttttgcttttattgccaagaggtgcttgttttaaaagtatgtttaataaaatgaaattctaaagttagaagtgttcttaagttgatatttactctcttggtcttggtagccctattatatcattgcagacacagtgttgtgcatgtgtgtattgtatatgtgcaccagcatcaaacattgtgtatctggaaggaaagcagcatgttccagttctaaaatgcagttaccagacccatcactttaaatccttaaagttagaagctagcaaattttctgtagtgcagaacgtttattgctgtttttgtgtttgaatagtataatgtttgatgcctctcttctgcaaagcgtatcatctcattgactgtgaatctgtatattattctgatggatacagataatgatcttttctcttgtgaggtatcttcatttatgcactgtccaaaaatagccatgtgtaagagtctttctgtatgacgaactacatggaaaagacttctgtggacataattctgaccgaaacccatgaagttacttcagtataagaagaacgttacacggaaatcaccaaatattttgcaactttatttcttctgacatggagtgaacatcaataggaatactttcaaagaaaatgaaaacacagaagcaaagagaaatgtggcacttcacattttaaactacagatggacttggtttgagggagggggaatcacagatttggtgctaagttaattagaaactggcagcgttttacagtagtacaccagcctggatgttttttctaaaatgtttacctgggagagctggggtttgtttgtgaggagaaagagtactgtggaaaacctctgcttgagtaccatgtggccaggcctatgtggatggctactccgtgctgtgcggcttcaccagcggttgggattggcccagcttggagtgcttgtgtggtccaacctcagtctggccccatagtgacttttgccccatgattctgcttcactgttggaatcctctttgaagttccccctctctttgctaaagcagtgaaggaagagaacagagacaaactctttggactgtgaaagagaaggtagagaattccaggcaacagtctgaccaagggtgtaaaccagtttattatatatatatttttcctttgaattaaaacctatgagtgactacaaaaaggattagactgagccgaataaaaaaaaaaaaaaaaaaaaaaaaaaaaaaaaaaaaa

SEQ ID NO: 18 - protein, 391 aa, Homo sapiens
MDQCVTVERELEKVLHKFSGYGQLCERGLEELIDYTGGLKHEILQSHGQDAELSGTLSLVLTQCCKRIKDTVQKLASDHKDIHSSVSRVGKAIDKNFDSDISSVGIDGCWQADSQRLLNEVMVEHFFRQGMLDVAEELCQESGLSVDPSQKEPFVELNRILEALKVRVLRPALEWAVSNREMLIAQNSSLEFKLHRLYFISLLMGGTTNQREALQYAKNFQPFALNHQKDIQVLMGSLVYLRQGIENSPYVHLLDANQWADICDIFTRDACALLGLSVESPLSVSFSAGCVALPALINIKAVIEQRQCTGVWNQKDELPIEVDLGKKCWYHSIFACPILRQQTTDNNPPMKLVCGHIISRDALNKMFNGSKLKCPYCPMEQSPGDAKQIFF

SEQ ID NO: 19 - DNA, 5565 bp, Homo sapiens
gcccctcgctgttctgccctttgtcttccacgcctgccccttcaactcctcttgccctgcaaaccctgggtcgccacctccacatcccttccacttctacctgcctgccttggcctgcattttattttcaaccaaggttgaaatggtagccccacgcaaaatccgatggtgtgtggaggaactgcccagtcattgcagtctgcacgccttcttaagtgccagatgcgagcaaggccgaccaggaactgccgatgggcttcacctgtcagactggactgccacctgccacacattcattgaactttcaataagcactgccgctgtgcttggcgttttgctgggctccagaaataaaaaattcagagaggccaaagttcttcagggatgcagacaggtgatagaaccatcatttattgagcacttgctaagtgctaaatactcttacctgcgttatcttgtgtaatcctcacaaccacactcaacgacggcctattgaatgctctctaggtgtctggcagtgttccaaacagtttatgtgtattactttgtttgatcttcccaaccaccaatgagcggatattcaactcctttgtttacactgagaaaatctcaaatggagaaagtgaagtacagcagctagccaaaaaaatccgagagaagttcaaccgttacttggatgtggtcaatcggaacaagcaagttgtagaagcatcctatacggctcacctaacctctcccctaactgcaattcaagactgctgtactatcccaccttccatgatggaattcgatgggaactttaataccaatgtgtctagaacaattagttgtgatcgactttctactactgttaatagccgggccttcaatccaggacgagacttaaattcagttcttgcagacaacctgaaatccaaccctggaattaagtggcaatatttcagttcagaagaaggaattttcactgttttcccagcacacaagttccggtgtaagggcagctacgaacaccgcagtagatttctgtgttaactgtggcagataccgtccggacttgctcactagaccagtgctataagaccttcttgtctccagccaccagtgagacaaaaaggaaaatgtccacctttgttagcagcgtgaagtcttcagacagtcctacccagcacgcagtgggattccaaaaggcatttcagctgattcgaagtacaaacaataacacaaagttccaagcaaatacagacatggtcatcatttacctgtcagctggcattacatcaaaggactcttcggaagaagataaaaaagcgactctccaagtcatcaatgaagaaaatagctttctaaacaactctgtaatgattctcacctatgccctcatgaacgatggggtgactggtttgaaagagctggcttttctgagggatctagctgaacagaattcagggaagtacggtgtgccagaccggacggccttgcctgtgattaagggcagcatgatggtgctgaatcagttgagcaacctggagaccacagtgggcaggttctacacaaaccttcccaaccggatgattgatgaagccgtcttcagcctgcccttctctgatgagatgggagatggtttgataatgactgtgagtaaaccctgttattttggaaacctacttctgggaattgtaggtgtggacgtgaatctggcttacattcttgaagacgtgacgtattaccaagactctttggcttcctatacttttctcatagacgacaaaggatatacacttatgcacccatctcttaccaggccatatttattgtcagagcccccacttcatactgacatcatacattatgaaaatattccaaaatttgaattagttcggcaaaatatcctaagcctccctctgggcagccagattatcgcagtccctgtgaactcatccctgtcttggcacataaacaagctgagagaaactggaaaggaagcctacaatgttagctatgcctggaagatggtacaagacacttcctttattctgtgtattgtggtgatacaaccagaaatacctgtgaaacaactgaagaacctcaacactgttcccagcagcaagctgctgtaccaccggctggatctccttggccagcccagtgcttgcctccacttcaaacagctggcaaccctagaaagtcccaccatcatgctgtctgctggcagcttttcctccccctatgagcacctcagccagccagagacaaagcgcatggtagagcactacaccgcctatctcagcgacaacacccgcctcattgctaacccgggcctcaaattctctgtcagaaatgaagtaatggctaccagccacgtcacagatgaatggatgacacaaatggaaatgagtagcctgaacacttacattgtccgccgttacatagcaacacccaatggcgtcctcagaatttatcctggttccctcatggacaaagcatttgatcccactaggagacaatggtatctccatgcagtagctaatccagggttgatttctttgactggtccttacttagatgttggaggagccggttatgttgtgacaatcagtcacacaattcattcatccagtacacagctgtcttctgggcacactgtggctgtgatgggcattgacttcacactcagatacttctacaaagttctgatggacctattacctgtctgtaaccaagatggtggcaacaaaataaggtgcttcataatggaggacaggggttatctggtggcgcacccgactctcatcgaccccaaaggacatgcacctgtggagcagcagcacatcacccacaaggagcccctggtagcaaatgatatcctcaaccaccccaactttgtaaagaaaaacctgtgcaacagcttcagtgacagaacggtccagaggttttataaattcaacaccagccttgcgggggatttgacgaaccttgtgcatggcagccactgttccaaatacagattagcaaggatcccaggaaccaacgcgtttgttggcattgtcaacgaaacctgcgactctcttgccttctgtgcctgcagcatggtggaccgactctgtctcaactgtcaccgaatggaacaaaatgaatgtgaatgtccttgtgagtgccctctagaggtcaatgagtgcactggcaacctcaccaatgcagagaaccgaaaccccagctgcgaggtccacctggagccggtgacatacacagctattgaccctggcctgcaagatgctcttcaccagtgtgtcaacagcaggtgcagtcagaggctggaaagtggggactgttttggggtgctggattgtgaatggtgcatggtggacagtgatggaaagactcacctggacaaaccctactgtgccccccagaaagaatgcttcggggggattgtgggagccaaaagtccctacgttgatgacatgggagcaataggtgatgaggtgatcacattaaacatgattaaaagcgcccctgtgggtcctgtggctggagggatcatgggatgcatcatggtcttggtcctggcggtgtatgcctaccgccaccagattcatcgccggagccatcagcatatgtctcctcttgctgcccaagaaatgtcagtgcgtatgtccaacctggagaatgacagagatgaaagggacgacgacagccacgaagacagaggcatcatcagcaacactcggtttatagctgcggtcatcgaacgacatgcacacagtccagaaagaaggcgccgctactggggtcgatcaggaacagaaagtgatcatggttacagcaccatgagcccacaggaggacagtgaaaatcctccatgcaacaatgaccccttgtcagccggggtcgatgtgggaaaccatgatgaggacttagacctggataccccccctcagactgctgccctactaagtcacaagttccaccactaccggtcacaccaccctacacttcatcatagccaccacttacaggcggccgtcacggtacacactgtcgatgcagaatgctaacaatctcctcacctccacgccaagatgagatctgggagccacagaatgttctggaaagaaaaagaaccggcttaaaacccacagcaagagacctcccttgtgtttgtgctttgtgcagagttgtttgagtcatttcctgcctgtcgacatggttaaaaacgagagaaacaacaacacagtcacatttgtgaagatgtgaggctggttctgaaatggaggggaaataagcctgatgaacagacctgccataacactaatggaaggtaacagaaggcgaacctccaaacacagagacggaacctgcaagtgaagctgagccagaggaatgttccaaagagccagaagcattcagctctccttaactggaagagagaaaaatctgctcacccagagactggaatgtggcacatgcagatacaaatgtgtgcattgaagatttcgctttgtttcttagcggtacctggataccacagttgctgtatggaactcatgttatgctctaaacgatgcatctcagaatttctaagtaaaggattatttttctactatttattgaactttcaaacattctcaaactttggggaaaaggaaaggaaacacaggagaagttttcagcagttgccccgagctgttttgtgtgtaatgaagtggttctttgattaaggagctctatttcttatttaactgatatcccactgccccactccacaaaataggaaaatgaagaaatctttctctctgacttgtttacatcatttcacggaaacacatctttgtttgtaatgcagtattctttctctgtgtttgacagagatggggaggggcagaggaatttaagaggttttaaaagaaatgttatgtttcttatgacttgtttccactcctcgtacaatgctattcttaggtttctacgaaacctaatgttagaaccgcatcctttcagctaagggagggttggatttattttccttgttttagagactacaaatttttaaatatcccattttgactgagaatattgacatataagggaagaagttttctaaattgtgaaagtctggttcttaattaaagaattttttttttaatatcacggttaaaagctgctgccagttagccaagacattatccaccaaattgctttgtgatttatacagggattaatcaaatctggctactataacatggggcattgtaactttaaagtagtgttttaattacagtgatgtattttagactcacattttgtgattcaaatatgttataaaggcattcttgcaccatggtaaagaatgtgtgtggtaaatctccgtttatatgtagttggaaaaaattcactgaataatgttttaatgatagggtattatgatacaatgtaaaaaacaattggttcttcagcagtacagaaagtaaactatatatgtgctatcaggaaaccccttcatactgtgtataaaattgcaatctagtgaaataaactgtatgcaatggaaaaaaaaaaaaaaaaaca

SEQ ID NO: 20 - protein, 978 aa, Homo sapiens
MSTFVSSVKSSDSPTQHAVGFQKAFQLIRSTNNNTKFQANTDMVIIYLSAGITSKDSSEEDKKATLQVINEENSFLNNSVMILTYALMNDGVTGLKELAFLRDLAEQNSGKYGVPDRTALPVIKGSMMVLNQLSNLETTVGRFYTNLPNRMIDEAVFSLPFSDEMGDGLIMTVSKPCYFGNLLLGIVGVDVNLAYILEDVTYYQDSLASYTFLIDDKGYTLMHPSLTRPYLLSEPPLHTDIIHYENIPKFELVRQNILSLPLGSQIIAVPVNSSLSWHINKLRETGKEAYNVSYAWKMVQDTSFILCIVVIQPEIPVKQLKNLNTVPSSKLLYHRLDLLGQPSACLHFKQLATLESPTIMLSAGSFSSPYEHLSQPETKRMVEHYTAYLSDNTRLIANPGLKFSVRNEVMATSHVTDEWMTQMEMSSLNTYIVRRYIATPNGVLRIYPGSLMDKAFDPTRRQWYLHAVANPGLISLTGPYLDVGGAGYVVTISHTIHSSSTQLSSGHTVAVMGIDFTLRYFYKVLMDLLPVCNQDGGNKIRCFIMEDRGYLVAHPTLIDPKGHAPVEQQHITHKEPLVANDILNHPNFVKKNLCNSFSDRTVQRFYKFNTSLAGDLTNLVHGSHCSKYRLARIPGTNAFVGIVNETCDSLAFCACSMVDRLCLNCHRMEQNECECPCECPLEVNECTGNLTNAENRNPSCEVHLEPVTYTAIDPGLQDALHQCVNSRCSQRLESGDCFGVLDCEWCMVDSDGKTHLDKPYCAPQKECFGGIVGAKSPYVDDMGAIGDEVITLNMIKSAPVGPVAGGIMGCIMVLVLAVYAYRHQIHRRSHQHMSPLAAQEMSVRMSNLENDRDERDDDSHEDRGIISNTRFIAAVIERHAHSPERRRRYWGRSGTESDHGYSTMSPQEDSENPPCNNDPLSAGVDVGNHDEDLDLDTPPQTAALLSHKFHHYRSHHPTLHHSHHLQAAVTVHTVDAEC

SEQ ID NO: 21 - DNA, 3352 bp, Homo sapiens
agcgggcagggaccagatccgcgagcccgtcagcctgcgccatgggctgcgacggccgcgtgtcggggctgctccgccgcaacctgcagcccacgctcacctactggagcgtcttcttcagcttcggcctgtgcatcgccttcctggggcccacgctgctggacctgcgctgtcagacgcacagctcgctgccccagatctcctgggtcttcttctcgcagcagctctgcctcctgctgggcagcgccctcgggggcgtcttcaaaaggaccctggcccagtcactatgggccctgttcacctcctctctggccatctccctggtgtttgccgtcatccccttctgccgcgacgtgaaggtgctggcctcagtcatggcgctggcgggcttggccatgggctgcatcgacaccgtggccaacatgcagctggtaaggatgtaccagaaggactcggccgtcttcctccaggtgctccatttcttcgtgggctttggtgctctgctgagcccccttattgctgaccctttcctgtctgaggccaactgcttgcctgccaatagcacggccaacaccacctcccgaggccacctgttccatgtctccagggtgctgggccagcaccacgtagatgccaagccttggtccaaccagacgttcccagggctgactccaaaggacggggcagggacccgagtgtcctatgccttctggatcatggccatcatcaatcttccagtgcccatggctgtgctgatgctgctgtccaaggagcggctgctgacctgctgtccccagaggaggcccctgcttctgtctgctgatgagcttgccttggagacacagcctcctgagaaggaagatgcctcctcactgcccccaaagtttcagtcacacctagggcatgaggacctgttcagctgctgccaaaggaagaacctcagaggagccccttattccttctttgccatccacatcacggccgccctggtcctgttcatgacggatgggttgacgggtgcctattccgccttcgtgtacagctatgctgtggagaagcccctgtctgtgggacacaaggtggctggctacctccccagcctcttctggggcttcatcacactgggccggctcctctccattcccatatcctcaagaatgaagccggccaccatggttttcatcaacgtggttggcgtggtggtgacgttcctggtgctgcttattttctcctacaacgtcgtcttcctgttcgtggggacggcaagcctgggcctgtttctcagcagcacctttcccagcatgctggcctacacggaggactcgctgcagtacaaaggctgtgcaaccacagtgctggtgacaggggcaggagttggcgagatggtgctgcagatgctggttggttcgatattccaggctcagggcagctatagtttcctggtctgtggcgtgatctttggttgtctggcttttaccttctatatcttgctcctgtttttccacaggatgcaccctggactcccatcagttcctacccaagacagatcaattggaatggaaaactctgagtgctaccagaggtaaaactgggtgaagaaggcaagagaagactttcagcctcttgatcaccagcacgaccatactgtttcagaaagctgggtggtggtggaggcgctatctcaatggctattcaagtcttctccactaaaacttggttgggtagaggaaattaaattgagtcctggtacctggtcaaaatcattagaagtttacctggcttctcaagttatcttcttccctggttcagactgttggtaagagctgtccagatacccagatgggaaggaaggagacagccgcgcgcttcactccatttgtcacctcatgcatggaccatactctgggtttgagatcattcttcattgaagtttgtaaaaataggttgaaattgtaaagctccatgatcattgctatgtagatatatttcaatttaagcaaaacaagctgcaagttattccctggcatgctcaaaggattttcgtgcttttcacttaatagtccaaagtctcttaaattcctgctgcagacatcaatagcttatctatattctcaaacaccaaaaggaaaagttgaatcttgctctctttggtatactaatgtagtggtatgctaagctggctcataccaacttagaaaagctgattgtaaaattttcattttgacagctggttattaaatgcagccattattaaaaatcaaatcatacaaacttataattaaatcaattacatttaaaacaaaggtaataaatattcaaagcatatcacttcctaatttgatcttgatgctcttgaggtaatttacgtccatggtacctgtgtggtggaattactatatatgatggtgtgctactgtgcaccttgtctcaactccactcttcgtgatagcatgttggtagcttgaaatcagcctggtgggagtattaccatggacactggcaaaagctacagatcccggagagccagtggttaaacatttaccagcataccactgctagtaatcaaggctaactggtccagaaatcgcccaggagaatgaaatggatgttccatttttttctactgacattgactagcatataaaaggtatagaaacagcactaagactttctgaaaatacctaatgaaaattttacatctttttttgtttttgtttttgtttttgagacagagtctcactctgttgccaggctggaatgcagtggcgtcatctcggctcactgcaacctccgcctcccagtttcaagcgattctcctgcctcagcctcccaagtagctgtgactacaggcgcatgccatcacgcacagctaattttttgtatttttagtagagacgggatttcaccgtgttagccaggatggtcttgatctcctgacctcatgatctgcccgtctcggcctcccaaagtgctgggattacaggcgtgagccactgcacccagccaaaaattttacatcttttatagagggaaaaaaactctttataccatggcaaggccttttctttcacaaaaagctgggcctactgaacaattcaagctgtgcagtagtagactgaaagcaggatttgttgaggagttacagctcctgtccagagcaaatcctgtagtgatacaaggagaatgtaaacttgccagcttagacagggatcagtcctgagactgctggcagtagcaaatggctattagagtaactgtataatggttttgcctgcactttctctatgtatatacaaatgtacatgtataaatataaaaattaagtgatcatgaaagaaaaaaaaaaaaaaaa

SEQ ID NO: 22 - protein, 403 aa, Homo sapiens
MALAGLAMGCIDTVANMQLVRMYQKDSAVFLQVLHFFVGFGALLSPLIADPFLSEANCLPANSTANTTSRGHLFHVSRVLGQHHVDAKPWSNQTFPGLTPKDGAGTRVSYAFWIMAIINLPVPMAVLMLLSKERLLTCCPQRRPLLLSADELALETQPPEKEDASSLPPKFQSHLGHEDLFSCCQRKNLRGAPYSFFAIHITAALVLFMTDGLTGAYSAFVYSYAVEKPLSVGHKVAGYLPSLFWGFITLGRLLSIPISSRMKPATMVFINVVGVVVTFLVLLIFSYNVVFLFVGTASLGLFLSSTFPSMLAYTEDSLQYKGCATTVLVTGAGVGEMVLQMLVGSIFQAQGSYSFLVCGVIFGCLAFTFYILLLFFHRMHPGLPSVPTQDRSIGMENSECYQR

SEQ ID NO: 23 - DNA, 1893 bp, Homo sapiens
gtgctgttcctgttctgtgtggccctataccaggtgcactatgaatggtggaggaaaattctaaaatatttttggatgtcagtggttatttacactatgctggtgcttatctttatatacacatatcagtttgaggacttcccaggcctgtggcaaaatatgactggattgaaaaaagaaaagcttgaggatcttggcttaaagcagtttactgtggctgaactattcactcgcatattcatcccaacctcctttctgctggtgtgcattttacacctgcactacttccatgaccggttccttgaactcacagacctcaagtccattcccagcaaagaagacaacaccatctacagccatgccaaagtcaatggtcgcgtatatctaataataaatagcatcaagaaaaaattaccaatccaccaaaatgaactggcccacccggaaggaagcctcccggacctcaccatgatgcatctgactgccagcctggagaagccggaggtgaggaagttggctgagcctggggaggagaagcttgagggctactctgaaaaagcccagaagggtgatcttgggaaagacagcgaggagtcagaggaggacggagaggaagaggaggaatccgaggaggaggaagaaacatcagacttaaggaacaaatggcacctggtgattgaccgcctcactgtgctcttcttaaaattcctggagtattttcacaagctgcaggtgttcatgtggtggattttggagttgcacatcatcaaaatcgtttcctcttacattatctgggtttctgtgaaagaggtgtctctgttcaactatgtatttttgatttcttgggcttttgctctgccgtacgccaagctgcgccgtctggcttcaagtgtctgcacggtctggacgtgtgtgatcatcgtctgcaaaatgtcgtaccagcttcaaaccattaagcctgagaacttctctgttaactgttccttgccaaatgaaaatcaaacaaacatcccctttaatgagttgaacaagtctctgctctacagcgctcctatcgatcctacagagtgggtcggcctgcggaagtcttcgcctctgctagtctacctgaggaataacctcctgatgctggctatcctggcctttgaagtcaccatttaccgccatcaggaatactatcgaggtcgaaataacctgacggcccctgtgtctagaactatctttcatgacattacaagactacatctagatgatggacttattaattgtgccaaatatttcattaattacttcttttacaagtttggtctggagacctgtttcctaatgtcagttaacgtcattggccagcgaatggatttctatgccatgatccacgcctgctggctgatcgctgtcttatatagacgcagaaggaaagccatcgcagagatctggcccaagtactgctgcttcctggcatgcatcatcaccttccagtatttcatctgcattggcatcccacctgctccttgccgagattacccgtggagattcaagggtgccagcttcaatgacaacatcataaagtggctgtacttcccagatttcattgtgcggcccaaccctgtgtttctcgtctatgacttcatgctgcttctgtgtgcctccttacaacggcagatttttgaggatgagaacaaggctgcagtgcgaatcatggcaggtgacaatgtcgagatctgcatgaatcttgatgcggcctccttcagccaacataaccctggtgccaccactgcaacttttgtattcacaggcatcccatctccatcacagataaaatcttaggagataaacacattcaaaaaggaatgagataaaaagaataaggcaataaatgttgattggaacctc

SEQ ID NO: 24 - protein, 583 aa, Homo sapiens
MSVVIYTMLVLIFIYTYQFEDFPGLWQNMTGLKKEKLEDLGLKQFTVAELFTRIFIPTSFLLVCILHLHYFHDRFLELTDLKSIPSKEDNTIYSHAKVNGRVYLIINSIKKKLPIHQNELAHPEGSLPDLTMMHLTASLEKPEVRKLAEPGEEKLEGYSEKAQKGDLGKDSEESEEDGEEEEESEEEEETSDLRNKWHLVIDRLTVLFLKFLEYFHKLQVFMWWILELHIIKIVSSYIIWVSVKEVSLFNYVFLISWAFALPYAKLRRLASSVCTVWTCVIIVCKMSYQLQTIKPENFSVNCSLPNENQTNIPFNELNKSLLYSAPIDPTEWVGLRKSSPLLVYLRNNLLMLAILAFEVTIYRHQEYYRGRNNLTAPVSRTIFHDITRLHLDDGLINCAKYFINYFFYKFGLETCFLMSVNVIGQRMDFYAMIHACWLIAVLYRRRRKAIAEIWPKYCCFLACIITFQYFICIGIPPAPCRDYPWRFKGASFNDNIIKWLYFPDFIVRPNPVFLVYDFMLLLCASLQRQIFEDENKAAVRIMAGDNVEICMNLDAASFSQHNPGATTATFVFTGIPSPSQIKS

SEQ ID NO: 25 - DNA, 2763 bp, Homo sapiens
agccgggcagctgcagcggagccgcggagcgggcggcggggcccaggctgtgcgcttggggagcgcggaatgtgaggcttggcgggccgcagcacgctcggacgggccaggggcggcgacccctcgcggacgcccggctgcgcgccgggccggggacttgcccttgcacgctccctgcgccctccagctcgccggcgggaccatgaagaagttctctcggatgcccaagtcggagggcggcagcggcggcggagcggcgggtggcggggctggcggggccggggccggggccggctgcggctccggcggctcgtccgtgggggtccgggtgttcgcggtcggccgccaccaggtcaccctggaagagtcgctggccgaaggtggattctccacagttttcctcgtgcgtactcacggtggaatccgatgtgcattgaagcgaatgtatgtcaataacatgccagacctcaatgtttgtaaaagggaaattacaattatgaaagagctatctggtcacaaaaatattgtgggctatttggactgtgctgttaattcaattagtgataatgtatgggaagtccttatcttaatggaatattgtcgagctggacaggtagtgaatcaaatgaataagaagctacagacgggttttacagaaccagaagtgttacagatattctgtgatacctgtgaagctgttgcaaggttgcatcagtgtaagactccaataattcaccgggatctgaaggtagaaaatattttgttgaatgatggtgggaactatgtactttgtgactttggcagtgccactaataaatttcttaatcctcaaaaagatggagttaatgtagtagaagaagaaattaaaaagtatacaactctgtcatacagagcccctgaaatgatcaacctttatggagggaaacccatcaccaccaaggctgatatctgggcactgggatgtctactctataaactttgtttcttcactcttccttttggtgagagtcaggttgctatctgtgatggcaacttcaccatcccagacaattctcgttactcccgtaacatacattgcttaataaggttcatgcttgaaccagatccggaacatagacctgatatatttcaagtgtcatattttgcatttaaatttgccaaaaaggattgtccagtctccaacatcaataagtgttgtaaacaattactgagacacggagcactgttaactgaaattcttctattccttcagctcttcctgaaccgatgactgctagtgaagcagctgctaggaaaagccaaataaaagccagaataacagataccattggaccaacagaaacctcaattgcaccaagacaaagaccaaaggccaactctgctactactgccactcccagtgtgctgaccattcaaagttcagcaacacctgttaaagtccttgctcctggtgaattcggtaaccatagaccaaaaggggcactaagacctggaaatggccctgaaattttattgggtcagggacctcctcagcagccgccacagcagcatagagtactccagcaactacagcagggagattggagattacagcaactccatttacagcatcgtcatcctcaccagcagcagcagcagcagcagcagcaacagcaacagcagcagcagcaacagcaacagcagcagcagcagcagcagcagcagcaccaccaccaccaccaccaccacctacttcaagatgcttatatgcagcagtatcaacatgcaacacagcagcaacagatgcttcaacaacaatttttaatgcattcggtatatcaaccacaaccttctgcatcacagtatcctacaatgatgccgcagtatcagcaggctttctttcaacagcagatgctagctcaacatcagccgtctcaacaacaggcatcacctgaatatcttacctcccctcaagagttctcaccagccttagtttcctacacttcatcacttccagctcaggttggaaccataatggactcctcctatagtgccaataggtcagttgctgataaagaggccattgcaaatttcacaaatcagaagaacatcagcaatccacctgatatgtcagggtggaatccttttggagaggataatttctctaagttaacagaagaggaactattggacagagaatttgaccttctaagatcaagtaagggacacttgaaggcttattttgcttcacagtaaaataacagctctattattattcagcaaggccaaagacttttgagaatgtgtatggaaaattctttttgtgcatttgagggcaaaattcaggccatcttcttatacatatactatcaaattatgttgtgtgcattagaaatcagttgcttgatagtagctattaaacccaatattgctgatagtatgctaatatcctaaaacttaaatattgcatatctatgaatgttaaattcagaatatctctaaaacatggaaaattgatgtttcaataaaaagggagacattttattattttgccttactaatgattttgcagctctgtttttctgcacactcaatagaaaatattgtgggtctgagatgccctttgaaaatgcctgaaagaaaacatgggctactacattatgttaatgttttgtaatgtccttttaatgagtggtgacataaaagggctgctttgttgtcctgatatggcaaaaattaaataaattctttttcactttaaaaaaaaaaaaaaaaaaaaaaaaa

SEQ ID NO: 26 - protein, 355 aa, Homo sapiens
MKKFSRMPKSEGGSGGGAAGGGAGGAGAGAGCGSGGSSVGVRVFAVGRHQVTLEESLAEGGFSTVFLVRTHGGIRCALKRMYVNNMPDLNVCKREITIMKELSGHKNIVGYLDCAVNSISDNVWEVLILMEYCRAGQVVNQMNKKLQTGFTEPEVLQIFCDTCEAVARLHQCKTPIIHRDLKVENILLNDGGNYVLCDFGSATNKFLNPQKDGVNVVEEEIKKYTTLSYRAPEMINLYGGKPITTKADIWALGCLLYKLCFFTLPFGESQVAICDGNFTIPDNSRYSRNIHCLIRFMLEPDPEHRPDIFQVSYFAFKFAKKDCPVSNINKCCKQLLRHGALLTEILLFLQLFLNR

SEQ ID NO: 27 - DNA, 4894 bp, Homo sapiens
cgaaaacggagaaaccccgggtccggcgagaggggctgtgacagtcggagtcccaagctgcggttcggctgctgccgagaactgcaaggtgtggaatatttctggcttctagtccaatgccaagtgtgtgacctgtggctacatgattccctgaaagataagaacaatgttatgttggggatattggtctctgggccaacctggtatcagcaccaacctgcagggaattgtggctgagccccaggtgtgtgggttcatatctgacagaagtgtcaaggaagtggcctgtgggggaaaccactctgtgttcctgctggaagatggggaagtttacacatgtggtttgaacaccaaggggcaactgggccatgagagggaaggaaacaagccagaacaaattggagctctggcagatcagcatatcattcatgtggcatgtggcgagtcccacagtctggccctcagtgaccgaggccagctgttttcttggggtgcagggagtgatggtcagctaggactcatgactactgaggattctgtggcagtgcccaggttaatacaaaagctgaaccagcaaacaatattacaagtttcctgtggcaactggcattgcttggctcttgcggctgatggccagttcttcacctggggaaagaacagccatgggcagcttggcttagggaaggagttcccctcccaagccagcccacagagggtgaggtccctggaggggatcccactggctcaggtggctgccggaggggctcacagctttgccctgtctctctcaggagctgtttttggctgggggatgaataatgccgggcagctagggctcagtgatgaaaaagatcgagaatctccatgccatgtaaaactcttacgcacgcaaaaagttgtctatattagttgtggagaagaacacacagcagttctcacaaagagtggaggtgtgtttacctttggcgctggttcctgtgggcaacttggacacgactccatgaatgatgaggttaaccctagaagagttctagagctgatgggtagtgaagtaactcaaattgcttgtggcagacaacataccctagccttcgtgccttcttctggactcatctatgcatttggttgtggagcaagaggtcaattaggaactgggcacacttgtaatgttaagtgcccatctcctgtcaagggttactgggctgcccacagtggccagctttcagcccgagctgatcgctttaaatatcatatcgttaagcagatcttctctggaggagaccagacttttgtactttgctccaaatacgagaattattctcctgctgttgacttcaggactatgaaccaagcacattataccagtttaataaatgatgaaaccatagcagtttggagacaaaaactctcagaacacaacaatgcaaatacaatcaatggtgttgttcagatattatcttctgcagcctgttggaatggaagttttcttgaaaaaaaaattgatgaacattttaaaacgagtcccaaaatccctgggattgacctgaactcaactagggtgttatttgagaagttaatgaactctcagcactccatgattctagaacagattttgaacagttttgaaagttgtctgattccccagttgtcaagctcaccaccagatgttgaagccatgagaatctatttaatactacctgagtttcccctactccaggattccaagtattatataacattgactattcccttggctatggccattcttcggctggatacaaaccccagcaaagtactagataactggtggtctcaggtatgcccgaaatatttcatgaagctggtaaacctctataaaggtgcagtcctttatctactgaggggaagaaagacattcttaattcccgtactgtttaacaattatatcacagcagctctcaaactcttggagaagttatataaggtaaatcttaaagtgaagcatgtggaatatgatacattttacattcctgagatttccaatctcgtggacattcaggaagactacctcatgtggttcttgcatcaagcagggatgaaggctagaccatcaataatacaggatactgtaacactttgttcctaccctttcatctttgatgcccaagccaagaccaaaatgttacagacagatgctgaactacagatgcaggtggcagtcaatggagccaacctgcagaatgtcttcatgcttctcaccctggagcctctgctggccagaagccccttcctggtccttcacgttcgcaggaacaaccttgttggagatgccctaagagagctgagcattcattctgatattgatttgaaaaagcctctcaaagtaatctttgatggtgaagaagcagtggatgccggtggtgttacaaaggaattttttcttttgctgttaaaagaacttttgaatcccatctatggaatgtttacctactatcaagattcaaatctcttgtggttttcagacacgtgttttgtagagcacaactggtttcacttgattggtataacctgtggactagctatctacaactccactgtggtcgatctccacttcccattggctctctacaagaagttactcaatgtaaagcctggcttggaagacttaaaggagttgtcacccactgaaggaaggagtctccaagagcttttagattaccccggggaggatgtggaggagactttctgcctcaacttcacgatctgccgagaaagctatggagtgattgaacagaagaagctgatacctgggggagataatgtaactgtgtgcaaggataacaggcaggaatttgtggatgcttatgtgaattatgtcttccaaatctcagttcatgaatggtacacagccttctctagtggcttcctaaaggtgtgtggtggcaaagtacttgagctcttccagccttcagaactgagggctatgatggtggggaacagcaactacaactgggaagaactggaagagactgccatctacaagggagattactcggccacacatcccactgtaaaactattttgggaaacatttcatgagtttccattggaaaagaagaagaagtttctcttgttcctgacaggcagcgatcggattcccatctacggcatggccagtctgcagattgtcatccagtccacagccagcggggaggagtacttgccggtggcccacacttgctacaaccttcttgacctccccaagtacagcagcaaagagattctgagtgcccggctgacccaggcccttgacaactatgaagggtttagtttggcctgaggcttctcagcttgtccagtatttcccttcgttcctcagtgtccacattgaggcctatacagaaaatcatggggagtgatttctatttttttattgtctaagtgggttgggacttttaaatactgagcctggttgatgtgtttctgggattgtatagcagtaaacaacctttttgaaaaattagaggttggggatggggtgaaaaattggcccttgtatgggaggtgtttttgtttttgttttaaaccaaactacccagtattccttgcacttgtgaatgtgttgcactctgctggatgaaatggcagtggatttttaaactttaatttcccaaatgtctctctcagccctgatgttttctcacagtgcttccttgtccttctcttaacttctcattcctctataagaatgatttagactgacctgtccttttttatctgcgcatgcgagaacatcaccttcctctgtacacttggaaatgcctctggcttgttgcagccctcctttaacccaaaggaggaaaggactgcttcagaaactcccaattccaaaaagctgagtctgggtccattattttggcagaactcctaagaatttatgggagcctatataaacatatcttgcttttaaaaagttcttgagggaatagcaactttcccatggctgtgcctatttcctagaccttttaaaagatgtgcagagcagcttagcattcgttgcagctgagcctaattttttcttgctcatccttgtccctttgacaataaggttaattgatagacccaccacctcttgcactctcgcttttggagcaagttgcattaactattttgagtctctatattgtccaagaaaagtagaaataataaatttactttccctttttctatcaccttatgtcctctaccattttctccttcctcccttcccttattttctccttttcgtaccctgtgtcctccctgattttcctttcgtttcttctttattttatcccattctctgttacttgactcagtgctcccttcctctcctctccttctagtggatgcatgcagcctttttttcaatttttatttaaattgcaaaatttttactcagattttttttcctcttccctaattgctaagatttaaggacgttctttattatgaaactttatcacattcgaaatgtttgtttacagtgggattttaggggggattgtgtttaaatcaaatatatgtattttaaaaataatgacatgctcaaccttcctcatcatggagtaagaaaattctacatgattaaagaatccatgtaagtctaattttaaattcctagtaactagagaaaagacttatttatataaaatgaagtatttatgaactgtgataaagcatcaaatcttgatgaaggattgtagatttttgctttttctttttgtttttaaaacttattccaattgctaaattggtagtttttcagtctttataaatacaggattaaaaatatatatacagttatatgaaatgtttattttctatgtgtgtgcatatagttcaatattatgcaataaatttggtgttttaactt

SEQ ID NO: 28 - protein, 1050 aa, Homo sapiens
MLCWGYWSLGQPGISTNLQGIVAEPQVCGFISDRSVKEVACGGNHSVFLLEDGEVYTCGLNTKGQLGHEREGNKPEQIGALADQHIIHVACGESHSLALSDRGQLFSWGAGSDGQLGLMTTEDSVAVPRLIQKLNQQTILQVSCGNWHCLALAADGQFFTWGKNSHGQLGLGKEFPSQASPQRVRSLEGIPLAQVAAGGAHSFALSLSGAVFGWGMNNAGQLGLSDEKDRESPCHVKLLRTQKVVYISCGEEHTAVLTKSGGVFTFGAGSCGQLGHDSMNDEVNPRRVLELMGSEVTQIACGRQHTLAFVPSSGLIYAFGCGARGQLGTGHTCNVKCPSPVKGYWAAHSGQLSARADRFKYHIVKQIFSGGDQTFVLCSKYENYSPAVDFRTMNQAHYTSLINDETIAVWRQKLSEHNNANTINGVVQILSSAACWNGSFLEKKIDEHFKTSPKIPGIDLNSTRVLFEKLMNSQHSMILEQILNSFESCLIPQLSSSPPDVEAMRIYLILPEFPLLQDSKYYITLTIPLAMAILRLDTNPSKVLDNWWSQVCPKYFMKLVNLYKGAVLYLLRGRKTFLIPVLFNNYITAALKLLEKLYKVNLKVKHVEYDTFYIPEISNLVDIQEDYLMWFLHQAGMKARPSIIQDTVTLCSYPFIFDAQAKTKMLQTDAELQMQVAVNGANLQNVFMLLTLEPLLARSPFLVLHVRRNNLVGDALRELSIHSDIDLKKPLKVIFDGEEAVDAGGVTKEFFLLLLKELLNPIYGMFTYYQDSNLLWFSDTCFVEHNWFHLIGITCGLAIYNSTVVDLHFPLALYKKLLNVKPGLEDLKELSPTEGRSLQELLDYPGEDVEETFCLNFTICRESYGVIEQKKLIPGGDNVTVCKDNRQEFVDAYVNYVFQISVHEWYTAFSSGFLKVCGGKVLELFQPSELRAMMVGNSNYNWEELEETAIYKGDYSATHPTVKLFWETFHEFPLEKKKKFLLFLTGSDRIPIYGMASLQIVIQSTASGEEYLPVAHTCYNLLDLPKYSSKEILSARLTQALDNYEGFSLA

SEQ ID NO: 29 - DNA, 4363 bp, Homo sapiens
gagctggaattcccttcccgcctggcgccgcccgggagagcccgcgcgaggacgcccgccgcgctccgccggcccttttttggcgctgagggaaaggagaagggcagggccgccgcctcgcgggagcgggccccgggaggaggggccgccggagaggccgggcgagcgcgggcggccctcacctcgccgctcctcccgggccgccatccctcggcgccccgcccggaaccggcgcgcgcgtaggggctgggaggccggcgcgcagatctggcggtgagcgctgccgccccggggcccccagccatgtcggccgaggagatggtgcagatccgcctggaggaccgctgctacccggtgagcaagaggaagctcatcgagcagagcgactacttccgcgccctctaccgctccggcatgcgcgaggccctgagccaggaggccggcggcccggaggtgcagcagctgcgcggcctcagcgcgccgggcctgcggctggtgctggacttcatcaacgccggcggggcccgcgaaggctggctcctgggcccgcgcggggaaaagggcggcggggtggacgaggacgaggagatggatgaggtgagcctgctgtccgagctggtggaggcggcctccttcctgcaggtcacgtccctgctgcagctgctgctgtcccaggtgcggctcaataactgcctggagatgtaccgcctggcgcaggtgtacgggctgcccgacctgcaggaggcctgcctgcgcttcatggtcgtccacttccacgaggtgctgtgcaagccccagttccacctcctggggtctcctccccaagctccaggggatgtcagcctgaagcagaggctgagggaggcccggatgactgggactcctgtcctcgtggccctcggggacttcctggggggacccctggcccctcacccctaccagggggagcccccgtccatgctcaggtacgaggagatgactgagcgttggttcccgctggccaacaaccttcctcccgacctggtcaatgtcaggggctatgggtctgccatcctggacaactacctcttcatagtgggcgggtacaggatcactagccaggagatctccgctgcgcattcctacaaccccagcaccaacgagtggctccaggtggcctccatgaaccagaagaggtctaacttcaaacttgtggctgttaattcaaaactctatgccatcggagggcaggccgtttctaacgttgagtgttacaaccccgagcaggatgcgtggaattttgtggcgcccttacccaatcctctggctgagttctctgcctgtgagtgtaagggaaaaatttatgtcattggaggatacactaccagagaccggaacatgaacattttgcagtactgcccctcttccgacatgtggacgctctttgaaacatgtgacgtccacattcgcaagcagcagatggtgtctgtggaagagaccatctacatcgtgggggggtgtctccacgagctggggcccaaccgcaggagcagccagagcgaggacatgctcaccgtgcagtcctacaacaccgtcacccgccagtggctctacctcaaggagaacacgtccaaatcgggtcttaacttgacttgtgcgctccataacgacggcatctacatcatgagcagagacgtcaccctgtcgaccagcttggaacaccgagtgttcctcaagtacaacatcttttcagatagttgggaagcatttcggcgttttccagcttttggacataacttgctggtttcttctctttatctgcccaataaagcagaaacatgactgaattgaattggtagatgaaaaaaacctggttcaagttttttttaaaatgtggtgtcccattccaagggagaccaattcctaaagggtaaagaagggttaaagtaggtcacatatatacagtagcagctgtaaataagcttacttgaactaattacttgaaaactggtggaaaaaagagaccaactttgttattttttaaattattgatttttaaattatgggagcaaatccatctccaaacatgatactcttgggccaatgacggatcatcaaaatgtagattcattgtgtctgtgtggtaaagggccaaagtcaataattgggacaaatggtaaagatgattttaaaatctgttgtatttaggtcccttgacatatcattagcaccttaagtgagaggttttctttttcttatcagtgtccttgacaagcattcatattaacatattcattctctcttctccatcacccatacccacacatacatctagtatgtttagggttctgtgttcagatattttgtttgcacactactttttaggaaacttataatgatggagccctacagtgaatccttaccagtgttcagtcctccccaaaacttgcataaaaatattcttagctatttttccagccttttttgcagttacaaatgctggataaatagactgataaaacctcttattttatttatgtggcaggttgcatatttatgtggacacataggttgcactgctaggcattctgtattgttttaaagaggatatattatttagaaaaatggacatatgaatattattaaaaattgagtcttaaatttaaatgggaagggaagaaaacaccaaataagaaaaaaatttgcctttaagagttacttttgttgaatgtatttgacttagttcttattttaaggctaaaaaaactttgtcatatttaagtcggcagctttctaatttaatcttagctttgtaattcatgttttgcttttgtggactaaggtaaggacagcatttaaatatttgtgctggagtgaggggaagaagctgttacagaagtggaatggtttctggtggtatttctgaagttaggtaaagtgctttacttgccttggttgttaattgaaatagaattcagctcaagcagttgttttcagtgagtgagttaacaataaaagcttctgccaatcatagatcaacattcctcaaaaatacatacactcatacttatggaaattttattttgattgaaaacactctgaaattatctgatattttgtaatggcatcagttaaactgtgagctggattccatctttctgcagtaacagcgggcagaaccatttcagttagcatgtgcctcactttaagaaagtcctgtaggggaaaaaaaaaactgtcagagcgaattcatgcattcaacaaacacttatgagtgcctggagtatgccgtccactgtgcagatgtaggacaggggctaatcacttagagttcttcagtttattagaggagtgactacaggttttctttacaaccatggcctctagtatacttaaaataagcgtccagttataaaaagcctatttgcacaactttttgggaatacttctattaactaaaatgaggtaagccattattttggacatagaaacagatctttgtatcttgtcaccaaataatcttacctctagtctacttacaactttgtaagggtttctaagctatgcccatttattcccaaatattaagcccttaaaaaaataaaacctctgaaccaaaatcttcccaggaatagtacttaatagattttaatgttaatttatattcattgtagaattgaatttccagtgacctgtaatttcattacatatattttaaaatctatttattttagatggtgttaacttttcgttgctgaagagcttaactttttaaaggctttgtcctatacatttagaagatgagtcagttgaattagtactggacaaacagttggagattagtttgcaaataagcatatcatatcaaatacaatattccagccaaccagttaattctcttcctggttacattcattgggtctatttgcctaatgttgacaattagattcttggaccaagtgagggtgtactctgatccacaaagctcttttataaaccaggtttgaggttgtgtgtgtgtgtgggggtgtgtgtgtgtgtgtgtgtgtatgtttgcatattatagctctctttaagacagacatctaaaaagattttggttattctgggctgccagcagtacgtgggaaaggtggaatgtgcaataggaacagatctgtgcattttgtgacatcgtccttcctgctgtgcacatcagctgtgatgacattttgagggtagttttaatttagctcggttctcttgttggtgccagtattacattgaagactacctatgatagaggcaatgttgctttgaaaatctcccttcaggcggaggttgcagtgagccgagattgtgccattgcactccagcctgggcgacagtgcaagactctgtctcc

SEQ ID NO: 30 - protein, 505 aa, Homo sapiens
MSAEEMVQIRLEDRCYPVSKRKLIEQSDYFRALYRSGMREALSQEAGGPEVQQLRGLSAPGLRLVLDFINAGGAREGWLLGPRGEKGGGVDEDEEMDEVSLLSELVEAASFLQVTSLLQLLLSQVRLNNCLEMYRLAQVYGLPDLQEACLRFMVVHFHEVLCKPQFHLLGSPPQAPGDVSLKQRLREARMTGTPVLVALGDFLGGPLAPHPYQGEPPSMLRYEEMTERWFPLANNLPPDLVNVRGYGSAILDNYLFIVGGYRITSQEISAAHSYNPSTNEWLQVASMNQKRSNFKLVAVNSKLYAIGGQAVSNVECYNPEQDAWNFVAPLPNPLAEFSACECKGKIYVIGGYTTRDRNMNILQYCPSSDMWTLFETCDVHIRKQQMVSVEETIYIVGGCLHELGPNRRSSQSEDMLTVQSYNTVTRQWLYLKENTSKSGLNLTCALHNDGIYIMSRDVTLSTSLEHRVFLKYNIFSDSWEAFRRFPAFGHNLLVSSLYLPNKAET

SEQ ID NO: 31 - DNA, 2501 bp, Homo sapiens
gttgctggctttttcaggagctgctcgctcgcagccagagacgctgctttttttttccgggttcggagccgttccggatgctttaggctgccggatgtctgatctccgaataactgaggcgtttctgtacatggattatctgtgttttagagcactttgctgcaagggaccaccacctgcacgaccagaatatgacctggtttgcataggcctcacaggttctggcaaaaccagtctgttgtccaaactctgcagtgaaagccccgataacgtcgtgtcgaccacaggttttagtattaaagcagtgccattccagaatgccatcttgaatgtaaaagaacttggaggggctgataacatccggaaatactggagccgctactaccaaggatctcaaggggtaatatttgtattagacagtgcctcttcagaggatgatttagaagctgctagaaatgagctgcactcagctcttcagcatccacagttatgcactttaccctttttaatattggccaatcatcaagacaagccagcagctcgctcagtacaagagatcaaaaaatattttgaacttgaaccacttgcacgtggaaaacgctggattctacagccctgctcactggatgacatggatgcactgaaagacagcttctctcagctgattaatttgttagaagaaaaagaccatgaagctgtaagaatgtgaaatctggcaaagaaaacaggctcccaaaaggccttgaacctatcatgttagtttctcattttaattttattttggtatcaagactatattggcttcagtatctattttcctctggtcatttcagactctcatctctctattaaagtgaatattctgtagatcagatgaattatcatcggcattaacgtcaagtacacactactgagagagaatttattctctgtttaccactaattatcttccccgcatgtctcttccttctgttgttctaagcatctcttctatactctgtgctgaaacggatggaattcatggcttatggaaatcaaatcccttttgaaggactcagaaagactcacaagaccttgatcattttctctgggtttgggagtggagtattatgtgactgggactccacaaaatatttatattattttcaagacctattgggggttttgttttattttatctaatttttttttttttttggtatactgagcttatagactatgagagaaaagtgagcaccaaaaacttcattaatcaccttatcaggaaggaatatcctgccatagtaaaattggctctgaagagcttagaggcaagttacacaatgttttcttgattacatctgaagtctcttctcttttaagagcaaagacattgtttcagtttctgagagtggttagaggaaatttaggtacaatgcatgcatcacagactgtatttgattgattcgagtcttcaaatattgtttaatggaatgatgagcctgcataaagtgaagctgcaatgtaacataggtatctttttctaaaaacttcaagtgtttgattaaaatagagaaaataatttttaaaataatagataaaaattttaaaaacagccagaaaaagtataatttatactggttagggtttaagatggcatggatttgacaaagataacaaagacagaatcatctatatattcattcattacgtgttactgtagtttattggaatctgttaatgccttcctctagtagttaaaggtttatcagtatttctattataaacctctattttcaggggtttcaatgtgcccttaaaattgtgctggggctgcagcaaataaataatattgggactcatattcagtctttggaactagttttttttccatttaatttaaatgacaaaattgcatagggataaatgtacagttttactaaggtttagcacctttgatcttttctaaatcaggaaaaaaatctatgcatattgaggttcacagatgaaatattctttggcatactttgtagactttgaccacttaaaatactaagtggccaaatgataaaaaaaactgctccctgcacatgagtgtaaaaacatgtttgtaattttaagtaaaatggactgatacccaaataccacagactcacactatattacagaatgatgtaatacctggtaattctccagaagatagacttcagacccttaatgccatttaaatatagtttttgtggtttaatcttatgagtagattgtaatgaaatcattacctttgaaacatcaaacacttctttaagctttttttttttaatgtcatgaatcaggaaggatacatagtgattcgtatacaattttgtctgcagcattcttgaatatttttaacactgtgtaatccctgagcagcacaaaacttttattccgatgaccagttacacaattcctgtttttattttgtcctgttgtgctcatcttctatgaaaaataaatgattattaggcaaaaaaaaaaaaaaa

SEQ ID NO: 32 - protein, 204 aa, Homo sapiens
MSDLRITEAFLYMDYLCFRALCCKGPPPARPEYDLVCIGLTGSGKTSLLSKLCSESPDNVVSTTGFSIKAVPFQNAILNVKELGGADNIRKYWSRYYQGSQGVIFVLDSASSEDDLEAARNELHSALQHPQLCTLPFLILANHQDKPAARSVQEIKKYFELEPLARGKRWILQPCSLDDMDALKDSFSQLINLLEEKDHEAVRM

SEQ ID NO: 33 - DNA, 2334 bp, Homo sapiens
ctttatggcgtgggagaggccacagcccggactccatcgactcccccggctcttagactaaaatcatgcccaagttcaaacaacgaagacgaaagctaaaagccaaagccgaaagattattcaaaaaaaaagaagcctctcactttcagtccaagctaattacacctcctcctccaccaccctcaccagaaagagtcggtatttcttcaatagatatatctcaaagcagaagctggctaacatcatcctggaacttcaattttcctaacatcagagatgcaataaaactttggacaaatagagtatggtctatatacagctggtgccagaactgcataacccagagtttagaagtattgaaagacaccatctttccatctcgtatctgccaccgagaactttacagtgtaaaacaacagttttgcattttggaaagtaaattatgcaagcttcaggaagcactgaagaccatctcagaaagttcttcctgtccaagctgtggtcaaacatgtcacatgagtggtaaacttacaaatgtgcctgcctgcgttctgatcacccctggagactccaaagctgtgcttcctcccacactgccacagccagccagccattttcctcctcctcctccacctccacctctgccacctcctccaccaccactagcacctgtgttgctcagaaaacccagtctcgctaaagcacttcaggctggaccattaaaaaaagatggacccatgcagataacagttaaagatctgctgactgtaaaattaaagaagacacagagtttagatgaaaagaggaagcttataccatcgccgaaagcacggaatccactagttaccgtctctgacttgcagcatgttaccctgaaacctaactccaaagtgttatcgactcgagttacaaacgtcttaatcactcctggaaaaagtcagatggatctgcggaaactgcttagaaaagtcgatgtagagaggaggccaggtggaacccctcttaccaataaggaaaatatggaaacaggaacaggactgactccagtgatgacgcaggccttaaggagaaagtttcagctggctcaccctagaagcccaactccaactctgccactttctacaagcagctttgatgaacaaaactgatgccaactctgcctcactccatgagatgatccataacaaatacagataaaaacacccagctgggtgcagtggctcgcacctgtaatcccagcactttgggaggctgaggcaggtggatcacctgaggtcgggagtttgagaccagcctggccaacatggtgaaatcccatctctactgaaaatacagaattggccgggtgtggtgtcacgcgcctgtggtcccagttacttgaacctgggaggcggaggttgcagtgagccgagattgcaacactgccactccagcctgggtgacagagcaagagaccctgtctcaaaaaaaaaaaaaaaaaaattaaatgggtagtgacgttaagagatatatatcagcttctagtaaaagtttttttttttaaacctgctagctacatttacattatgtaaaaataaatggaataatcactgagaataaagcagttgagtatttataacaataatattttatggggcgcttataatgtttataatattgtaaaccactgtgtactctattcatttaatgctaaatgacttgaccattcttgtgggataagagatcattaaaaaaatgctagggccgggcaccatggctcacgcctgtaatcccaggactttaggaggccaaggcaggtggatgacttgagctcaggagtttgagaccagcctgggcaacatgatgaaaactccgtctctaccaaaaatcaaaaaaattagccaggtgtgatgttgtgcgcctgtagtcccagctacttgtgaggctgaggtgggaggatggtttgggccccggaggtagaggttgcagtgagctgagattgtgccactgtattccatcctgggcaacagagccagaccctgcctcaaaaaaaaaaaaaaaaaaaaaaaggcctggcacagtggctcacgcctgtaatcccagcactttgggaggctgaggcgggcaaaacacttgaggtcagacgtttgagaccagcctggccaacatggtgaaaccccgtctctaccaaaaataaaaataaaaattatccaggcgtggtggcatgtgcctatagccccagctactcaggaagctagagcacgagaattatgtgcatcctggaggtgaaggttgcagtgagccaagatcacaccactccactccagtctggacaacagagtgagaccctgtgtc

SEQ ID NO: 34 - protein, 360 aa, Homo sapiens
MPKFKQRRRKLKAKAERLFKKKEASHFQSKLITPPPPPPSPERVGISSIDISQSRSWLTSSWNFNFPNIRDAIKLWTNRVWSIYSWCQNCITQSLEVLKDTIFPSRICHRELYSVKQQFCILESKLCKLQEALKTISESSSCPSCGQTCHMSGKLTNVPACVLITPGDSKAVLPPTLPQPASHFPPPPPPPPLPPPPPPLAPVLLRKPSLAKALQAGPLKKDGPMQITVKDLLTVKLKKTQSLDEKRKLIPSPKARNPLVTVSDLQHVTLKPNSKVLSTRVTNVLITPGKSQMDLRKLLRKVDVERRPGGTPLTNKENMETGTGLTPVMTQALRRKFQLAHPRSPTPTLPLSTSSFDEQN

SEQ ID NO: 35 - DNA, 6743 bp, Homo sapiens
cgacggcggcggccgtcctcatcccggcgcttgagaggacgcggggctgcgcaaatggcttgtcccgctctcggtctggaagctcttcagcccctgcagcccgagccgccccccgagcccgccttctccgaggcgcagaagtggattgagcaagtaactggcagaagttttggtgataaagattttcggacaggtttagaaaatggaatcctcctctgcgagttgctgaatgctataaagccaggacttgttaaaaagatcaatagattgcctacccccattgcaggactggacaatattatcttattcttgagaggttgtaaagagctcggccttaaagaatctcaactttttgacccgagtgacctccaggatacatccaacagagtaacagtcaagagccttgattatagtaggaagctgaaaaatgtattagttaccatttactggctgggaaaagcagcaaacagctgcacatcctacagcggaacgacactaaacctgaaggagtttgaaggattgttggctcagatgcgaaaggacactgatgacattgaaagtcctaaacgcagtatccgagacagtggctacatcgactgctgggattccgagcgcagcgactccctctctcctcctcgccacggcagagatgattccttcgacagcctggattcctttggctctcgctctcggcagacgccttcaccagatgtagtcctcaggggaagcagcgatgggagaggaagcgactctgaatccgacttgcctcatcggaagctgccagatgtgaagaaggatgacatgtctgcacggcggacttcccatggtgagccgaaatcagcagtgccttttaaccagtacctcccgaacaaaagcaatcagacggcctacgtccccgcgcctctgagaaagaagaaagcagagagagaggaataccgcaagagctggagtaccgccacctccccgctgggtggggagaggcccttcagctttcctgaaacgatagaggaagaggggagtgaagtggggtctgctggtgaagacaacccagctggccaaatgaatcctggttggaagccttccgatggtgggtgtgaattacctgacggcagtggtaaggagcacccttcttcagacggggctgtggtggcaccagctcccaagtctgaagaaaaagatgctgctgagatccaaaagcgcaaaaggctagagcaagctggaatcaaggtcatgccagcagcacagcgctttgccagtcaaaaacaattaagtgaagagaaagaagctattcgtgatattgtccttcgcaaagaaaactctttcctgacccaccaacatggcaacgattcagaggcagaaggtgaagttgtgtgtcgactgcctgatcttgagaaggatgactttgctgcaaggagagcaaggatgaaccaaaccaagccaatggtgccattaaatcaactcctctatggcccttatccaaagaaaggggcagagaaatcagatggcagcaaacagctctcaaagggaatctcaaaaaaaagaagtctagaatataaaagaaaccagggccacacagaagaggtgaagttgatagtgacctgtaacatgagggctcaggaaagtgaacctgtggaaggaggactcaggaaggtgccagatcttcacaaggatgacctggcccagcagagaattcagggcagccttgcccctcaccgcgagcccccgagcttcattacgctctccaacataacagaagctgacttggagacgtgggagagactcaaagtgtcagaaaaggcgagggatggagatgttcagcacatctgtgcttctgagccttccccagaaattaaagcagaaactgccattcgtgatgactttgccaaccgcaaagcaagggcctctaagaaagcttccagccccaggcaaaagtttgtgcactttgggccagtgacggagctagatcagcagaaatggaagcggcttagcattggcaaggctgggcctagagaggatgaagaagaagtcatctgtcatggcagcaagattcaaatggactctgtgtctcctgtctcagcggccacttccagcttaaagggccaccaaatatttaatcgacaaaatgactgcagaacaatgaattgtggccgaggtgactattgcagaagggcctcgtggctggctcctgtgccggagtctcaggaagagtgggtctgcagtttgggcgagtgcccgagggggacagaggaagttacatccaaacagctgccacaggatggcaaagaagaaacagaaagcgctccaagagattctgagaggctgtcaaaggcagaaagatcagaggacagcagccagccactggtgtgtcctctggcctctgagtgtgaggcttcagggacagaagagaagttggagaagatgacagctcctgcctggagtggcagtggactgaaaggccagcgaaagttggatgattcacgaaaagatgacatgatggccaggagaactgggatgtcccttagacacactggatccaacccaaaccagttccttccagttccatttgcaaagcaacaggatgtggaagaatcttctaaaggtctacccatgaaagatcagagatacggtccgagaactcctgtgtctgatgacgcagagagcaccagcatgtttgacatgcggtgtgaggaggaggccgcggtgcagccgcacagcagggcccgccaggagcagctgcagctgataaataaccagctgagggaagaggacgacaaatggcaagatgacctggctcgttggaagagtcgtagaagaagtgtttctcaggacttaatcaagaaagaggaagaaaggaaaaaaatggagaagttactggctggagaagatgggacaagtgaacgaaggaaaagcatcaaaacctacagagaaattgttcaagaaaaagagcggagagagagagagctgcatgaagcatataagaacgctcggtcccaggaggaggcagaggggatccttcaacagtacattgagaggttcaccatcagtgaggctgttctcgaacgcttggagatgccaaaaattctggaaagaagccattcaacagagccaaatttatcctccttcctgaatgaccccaatcccatgaaatacctgcggcaacagtcactgcctccacccaaattcactgccactgttgaaaccaccattgctcgtgccagtgttctggataccagcatgtcagcaggcagtgggtctccaagcaaaactgtcactcccaaagcagtgcctatgctgacacccaagccttactcccagcccaaaaattctcaagatgttctgaagacctttaaggtagacgggaaagtcagtgtgaatggagagacggttcatagagaggaggagaaggaaagagagtgtcccacggtggcacctgcccactccttaaccaaatcccagatgtttgaaggtgtggccagagtgcacgggtctccactggagctgaaacaagacaacggtagcatcgagatcaacataaagaagccaaactctgttccccaagagctcgcagcaaccactgagaaaacggaaccgaatagtcaagaggacaagaatgatggtggaaaatcaagaaaagggaatatagaacttgcctcatcagaaccacagcattttacaacaactgtgactcgatgcagcccgaccgtggcctttgtggaatttccctccagcccccagctgaagaatgatgtgtcggaagaaaaagaccagaagaaaccagaaaatgaaatgagtggaaaggtggagttggtgctgtcacaaaaggtggtaaagccaaaatctccagaacccgaagcaacgctgacatttccatttctggacaaaatgcctgaagccaaccaactacatttgccaaatctcaattctcaagtggattctccaagcagtgagaagtcacctgttatgacaccttttaagttctgggcatgggacccagaagaggagcgcaggcgacaggaaaaatggcaacaggaacaggaacgtttgctccaggagagataccagaaggagcaggacaagctgaaagaagagtgggaaaaggcccaaaaggaggtggaagaggaagaacgcagatactatgaggaggagcgtaagataattgaagacactgtggttccatttactgtttcttcaagttccgctgaccagctgtctacctcttcctccatgactgaaggcagtgggacaatgaataagatagacctgggaaactgtcaagatgaaaaacaagacagaagatggaagaaatcattccagggagatgacagtgacttattgctgaagactagggaaagtgatcgactggaggagaagggcagcctaactgaaggggccttggctcattctgggaaccctgtatcaaaaggagtccatgaagaccatcagctggataccgaggctggggccccacactgtggaacaaacccacagcttgctcaggatccatcccagaatcagcagacatcaaatccaacgcacagttcagaagatgtgaagccaaaaaccctcccgctggataaaagcattaaccatcagatcgagtctcccagtgaaaggcggaagaaaagtccccgagagcacttccaggctgggcctttctctccctgttctcccacccctcccggtcagtcaccaaacaggtctataagtggaaagaagctgtgctcttcctgtgggcttcctttgggtaaaggagctgcaatgatcatcgagaccctcaatctctattttcacatccagtgtttcaggtgtggaatttgtaaaggccagcttggagatgcagtgagtgggacggatgttaggattcgaaatggtctcctgaactgtaatgattgctacatgcgatccagaagtgccgggcagcctacaacattgtgacacggctttcaagcttccggatcactcaccatttctttactgagagtgtcccctggcaactgcttaacaaaatcccaagctcaggggcttctcagcatttacctaatttctgaaaggctcttctgaaaggtggtatctgttctttcgtagcacagtgtttatgtttttcctgtttattgttttggttttttttttttttttgcatttgcacagtatacacaaaagaatatggggttgtaatgatcctgaatagctcaaaaaaggttttagcatggtcaaacaggcttatggtttaaaatgtgttattctcttctttgggaattagctaaatgatgcaataaacctgttttgttttagaatgtctaggaattaaacactttatgtttacagaattgagctgcagaaagtgcaagacatgccaatttgagacacacggtcttctaagactgaaggataaatttaatgcatttcagaaactaaacatcacagcaagctctatctctgagctataatttgtttttaatgcaaagacactagtttgataatatatactgtaatcctgaaacatttgtgttacttacctttggaggtagaaattataccaataaattattgcaccgttagtattagattctgtgtaccttggaagttatgtcattaatataggctggttcatcaaataaagcaaaaccttgcaatatcagctagatttacactccgggacgttgcccaaaggtaggaagaaagcagagggaaatatttcagtcatcatttccaaagtcattatcaaaatctgtgaggaagtttaatcttccaaagagtcaatgtcagacatcaggcctctgttgcctgcttctctcgaggcactagattaggagtcttcaataagagacttaacatgaggtatatggaagatgaggcaccgagataagttcatcattaggtgtgagcactgctcacccttgctggcaagttctccttaagggcctgaagcacaggtgtccaaagaaaagcgttaagtccatcttaatagaatctatgtggtatatgatgtggtcagcccctggtctgtgatcagcaagaacctacagcacagattatgccctgcccacttcaatgaatacctactctcctccattctccatcactttttttgctatcaagaactccggaccttgcccatggagaagtttagagaggaactcttgtggagagctggtttattttctgccctgtgcgacgagtttcagctggccaagaaaggagtcaagttattaaaaagcatcacaatgtagatctccaggctggttttttgttttttgttgttaagactggggaaagggggactatttattctgccttaaatcaatggcaaataagtcaagatgacattttgtgaatgtagactatggatacactcctaatagattgatgtagtcataaaagggggtcaagtagatgtttttctgttatgtaagcaataatttttccgtgtcttattgagtatggctagcgattatttattacatgctagatgggttctttgcatgtgggttccatataggtgcagaaatttcctcagccactggagggatttcgaccatatttgtcatttggatgagctgttattagattgaaatctacacatcatttcattaaaaattgtgccttagaaaacgcaaagctgttgcacatggcgataaattatggatgcagtacattgaagagagatgaagtcacttccaagtttccaagacttctcatggaggtgtttgctgttttacaggaaaaaataaaaataaaaaaag

SEQ ID NO: 36 - protein, 1467 aa, Homo sapiens
MRKDTDDIESPKRSIRDSGYIDCWDSERSDSLSPPRHGRDDSFDSLDSFGSRSRQTPSPDVVLRGSSDGRGSDSESDLPHRKLPDVKKDDMSARRTSHGEPKSAVPFNQYLPNKSNQTAYVPAPLRKKKAEREEYRKSWSTATSPLGGERPFSFPETIEEEGSEVGSAGEDNPAGQMNPGWKPSDGGCELPDGSGKEHPSSDGAVVAPAPKSEEKDAAEIQKRKRLEQAGIKVMPAAQRFASQKQLSEEKEAIRDIVLRKENSFLTHQHGNDSEAEGEVVCRLPDLEKDDFAARRARMNQTKPMVPLNQLLYGPYPKKGAEKSDGSKQLSKGISKKRSLEYKRNQGHTEEVKLIVTCNMRAQESEPVEGGLRKVPDLHKDDLAQQRIQGSLAPHREPPSFITLSNITEADLETWERLKVSEKARDGDVQHICASEPSPEIKAETAIRDDFANRKARASKKASSPRQKFVHFGPVTELDQQKWKRLSIGKAGPREDEEEVICHGSKIQMDSVSPVSAATSSLKGHQIFNRQNDCRTMNCGRGDYCRRASWLAPVPESQEEWVCSLGECPRGTEEVTSKQLPQDGKEETESAPRDSERLSKAERSEDSSQPLVCPLASECEASGTEEKLEKMTAPAWSGSGLKGQRKLDDSRKDDMMARRTGMSLRHTGSNPNQFLPVPFAKQQDVEESSKGLPMKDQRYGPRTPVSDDAESTSMFDMRCEEEAAVQPHSRARQEQLQLINNQLREEDDKWQDDLARWKSRRRSVSQDLIKKEEERKKMEKLLAGEDGTSERRKSIKTYREIVQEKERRERELHEAYKNARSQEEAEGILQQYIERFTISEAVLERLEMPKILERSHSTEPNLSSFLNDPNPMKYLRQQSLPPPKFTATVETTIARASVLDTSMSAGSGSPSKTVTPKAVPMLTPKPYSQPKNSQDVLKTFKVDGKVSVNGETVHREEEKERECPTVAPAHSLTKSQMFEGVARVHGSPLELKQDNGSIEINIKKPNSVPQELAATTEKTEPNSQEDKNDGGKSRKGNIELASSEPQHFTTTVTRCSPTVAFVEFPSSPQLKNDVSEEKDQKKPENEMSGKVELVLSQKVVKPKSPEPEATLTFPFLDKMPEANQLHLPNLNSQVDSPSSEKSPVMTPFKFWAWDPEEERRRQEKWQQEQERLLQERYQKEQDKLKEEWEKAQKEVEEEERRYYEEERKIIEDTVVPFTVSSSSADQLSTSSSMTEGSGTMNKIDLGNCQDEKQDRRWKKSFQGDDSDLLLKTRESDRLEEKGSLTEGALAHSGNPVSKGVHEDHQLDTEAGAPHCGTNPQLAQDPSQNQQTSNPTHSSEDVKPKTLPLDKSINHQIESPSERRKKSPREHFQAGPFSPCSPTPPGQSPNRSISGKKLCSSCGLPLGKGAAMIIETLNLYFHIQCFRCGICKGQLGDAVSGTDVRIRNGLLNCNDCYMRSRSAGQPTTL

SEQ ID NO: 37 - DNA, 8456 bp, Homo sapiens
  misc_feature (3848)..(3848): n is a, c, g, or t
  misc_feature (5680)..(5680): n is a, c, g, or t
  misc_feature (5735)..(5735): n is a, c, g, or t
  misc_feature (5750)..(5750): n is a, c, g, or t
  misc_feature (5858)..(5858): n is a, c, g, or t
  misc_feature (8099)..(8100): n is a, c, g, or t
  misc_feature (8108)..(8108): n is a, c, g, or t
  misc_feature (8130)..(8130): n is a, c, g, or t
  misc_feature (8448)..(8448): n is a, c, g, or t
ctctcgcggcgcgaccgctcagtcacttcgcccagagacccggacctggtccgctggggagcaggcggccataaaccccctctctcccggttccctgacgccgcggcaggagctgttacaaacaccctgcggttggtctccgatgcccttcagtgaggtggggacgcctggaccctggtgagcgaaccccaagccaccccccaccccaactcagtgtcttcgccggcccccggcccgtacgcctgtctggtcgccatggctgaaaacacagagggggatctgaactccaacctgctccacgccccctaccacaccggggaccctcagctggacacggccatcgggcagtggctccgctgggataagaatcccaaaacaaaagagcagattgaaaacctgttacggaatgggatgaacaaggagctgcgagatcgtctttgttgccgaatgacttttgggactgcaggacttcgttctgccatgggggcagggttttgctatattaatgaccttacagtaatacagtcaacacaggggatgtacaaataccttgagagatgtttctcagacttcaagcagagaggctttgtggttgggtatgacactcggggtcaagtaactagcagctgcagcagccagaggcttgctaaactcactgctgcagtcttgctggccaaagatgttcctgtgtaccttttttcaagatatgttcctacaccttttgtaccatatgcagttcagaagctcaaagcagttgcaggtgtgatgattactgcctctcacaaccgcaaggaagacaatggatacaaggtttactgggaaactggtgctcagatcacatctcctcatgataaagaaattctaaaatgtatagaagaatgtgtggaaccctggaatggttcctggaatgataatttagtggataccagcccgctgaagagagaccctctgcaggacatttgcaggagatacatggaagatctgaaaaagatctgtttttacagggagttaaactcgaagaccaccttgaaatttgtgcacacatcttttcatggggtcggacatgactatgtgcagttggcttttaaagtgtttggttttaagcctccaattccagtaccagaacaaaaagatcctgatccagacttttctaccgttaaatgtccaaatcctgaagaaggagaatctgtgctggaactttccttgagactggcagagaaagaaaatgcccgggtagtgctagccacagatcctgatgcagacagactggcagcagcagaacttcaggagaatggttgttggaaagttttcacagggaatgagttggcagctttgtttggatggtggatgtttgattgctggaagaaaaataaatcaagaaatgctgatgtgaagaacgtttatatgttagccaccacagtctcttctaaaattctgaaggcaattgcacttaaagaaggatttcattttgaagaaacattaccaggttttaaatggattggaagtaggataatagacctcctggaaaatgggaaagaagtcctttttgcatttgaagagtctattggttttctctgtggaacttcagttttggataaagatggggtgagtgcagctgttgtggttgctgagatggcatcttacctggaaaccatgaatataacattgaaacagcaactggttaaggtttatgaaaaatatggttatcatatttcaaaaacttcctatttcttgtgttatgaaccacctaccatcaaaagtatatttgaaaggcttcgtaattttgattctccaaaagaatatccaaaattttgtggaacatttgctatattgcatgtacgggacgttaccactggatatgacagtagccagcctaataagaaatcagtgctgcctgtgagtaaaaacagccaaatgattacatttacttttcaaaatggctgtgttgctacccttcggacaagtggaacagaaccaaagataaagtattatgcagagatgtgtgcgtcacctgaccagagtgacactgctttactggaggaagaactgaagaaactcattgatgctctgatagagaattttcttcagcctagtaagaatggactgatctggcgttctgtttaggggtacaccaatatgtcatgacactgtgtgggcatatggaacagagcaaccgagaactgcatacattgaaccttgtgttagcattctctctctatctcatctggccgagtatctttttccttttattttctttctttttggtcaactggctagactaaacaaagtataagtttgaaatgaaatggtctagagggaaatgattcaaattttttcataattcaaacaaaagttattacactaaaagtattatagtaacgtattgtccttccgttaacagaaacatcacaattcaaaagtgagttttcttattatagtgtgattaagcctagttctgtatctcgtaattgtgtatagcatggtaaaagaaaaaaaaaaagacaggtaaatgttatataattgtagggttagttaaggaaattagtccataaaattggcaagaaaatggtactttccttgattgttatgaaaaagtgtcattgtgacatcacaggtattaatttacaaattagagtatttgaaataggatatttagcatttctgtagtttagaacagatgttacagtgtgtggttttggcatgtctgtttcaggtgcccttggtgcagtataaatttactgtatgtatttaaaattctgatgtttactgaacataaaacaatagtggcaacactcctattggctttgttcttatcaaatttctcagtgccttctcagggtctggtatgaacagaaaaaacagtgttcctgcatttaccataatttaatcagtcaatttgcagatagctaaggaaaatcttccaaaatctgtaggtgcttcctctatatcaagtgagtttactgattttaaaatcttaggaatataaataaagatcttaataaaagcaagcttaatgtagttaaggatcttcatattagaattgtgtcatttatcaaaccttttattacagaaaaaaatctgaactgagaaaggagctctgaatttatttgacagtctaacttttgaattacatttttgtaagtagatttatcttttattttatgaagtaatgttaattttactaagaataaaacatccaaattttaagtattatgaaatactaaaactacatgttttaaaacagaggtccagtttattctaacatcttgctgacttctgtagaatttctgattgcatgactattatttatcttcttttagtgtagaaaataattttttacaaaaagtcacttttgggctgggcgcagtggctcacgcctgtaatcccagcactttgggaggctgaggcaggtggatcacaatgtcaggagtttgagactagcctggccaatatggtgaaaccctgtctctactaaaaatacaaaaattagccgggtgtggtggtgggcacctgtagtcccagctactcgggaggctgaggcaggagaattgcttgaacctaggaggctgagattgcagtgagccgaaatcacgccactacactccagcctgggcaacagagggagactccatctcaaaaaaaaaaaaagtcacttttggctaatcatgcagtcttctgaaaaggtttaataaaaaaatgctgtttaggctgggcttggtggctcacatctgtaataccagcgctttgggaggctgaggcaggaggattgcttgaggccaggagtttaagttacagaactatgatcacactgccgcactgaagcctggatgacagagtgagaccttatntcaaaaaaaaaaaaaaaaagagagagagagagaaaaagaaatgctgtgtttaatcttcttaaaatgtatcttgtcctacatagttctcagaatacatatggggtatagttaggagataacaataacaagacctaaacaaattgcttatgggagatagcactgtaattagagaatttatttcatataaaataataatataaagtttgaactgattagaatgttccttaaatcatggtttcgtttgttgcagctgtcagctaacatttggttgagctcatataatccaaacctaattgtccaactattgtattgccactattgttaatttgttccttccttactttcttattgttggatcttatatgactgtatgactgtagataatagacagctagctaaatgtagagcaccatttttctcaatgagtttgttttcaaaggctttgcactggagaagatatgtgtagaatttttgttttatttttaaaatgtgtaaaacatcttttgttttatatgcttttaagaatgtagattgtaatttaggaaatggcatatattctgcaaagtggtataatgttgtatatatgaaaaacaggtatatttggtttagttctgtgtttcagtgactgataaacaaaaatgaagctaaaatgaatcaacggctcattcatagttactaaagccatgtcatgactgtcattctattatgaagaaaacagttttatttgagtgctttaatataatgcaacatttaagtcatcttaaggtgaaaagttattgaagtgttcttctctcaagttaacaaaattttacaaatgttcctgaagtgtatcgattgagggaattattttattctagcctgtcatgagtctgtctgagcatgatacttaacatgaacttgatgtagtaggtgcacatttgcctaacaggataaattcagtgatagcccaagctggtcaggagagacagaagtgctggctaggaggtctcagcctcgggcactacaatgtttatctttttctttttgaacaatattgtgaaaatgattgcattctcattgagcatgaaagtcaaaactggaattttctaaagctttggagaaaaggagagaattagtagggcaagatcaaagcatacattggaaataaacagtagaaataagcaaacaatataaagagacacgggaaatgcttctcgtgaagggcaaatgtaacaccataacagtagggaataaactgtgtcccacctgtttacttgtgtttatttgttgttatgatgagtatgcacctaccattttcctttattgtgatcactaaaatgaggccaaggtttaggaggtgagaaaaagtatcccttttacggattcagttcctgctaggatgttgtatcagaatcatgtgagaactcaggaagaggtagagcagaaataaaaaggaagaaaatgtagattagaaaataagacttctgagaaaaagttcaaggaataaagattgtttgactctggagaggagaaagctgttgagatagtttgctaagtttgctaataaagctttattgagtcttttattgaagggttcaaagtaagaaatactatgccatagtgttctccatctctagtgaagacagaaccagagggaataaggatcttttttttaaagacagcatggaaagtttaagatttaggaacttcttgagaaatcttcataatatcttctattcaaaatcttttttttatttttaagggaataaaaattttaaaaattaagagatatacagatgactttttaaggtccttctttccagcacattttttttaaaaactcaggttagaanaaaaatgtcataaagggcactaaaagtaacatttgggtttcactccatatttagnagaatttagctcacnaaacaatttttgtgtgttttgagtaactgatgcataagtgcaatgaataattttcctgcttcactgttatttttaatttaacctggcaaattaagtgagacttgaacnaaattttgttcatcttgaagaatgtatagtggcttaggttatttgaggttttcgttttgaatatatatgttgttattttgatgtagatacgttagcagatggagaggttctaagagattggatactttggaatgtctttatataggaaatagacctcccatttcttaagcaagttgcttctggaattgatgcaacaggggatattagctgtagtttaatggataagttgttctatgagctgctgctatcagtacttttttccacattttccattgatatttgtgatgcttgaaaaatccaaacttcacttggatacatttaattttatgcctttttctctctacctcccacttttcctaggcacccttgcacattttgttactgtgaaggactattgccctgttacctcactgataagaaatctcaactgtattcattcagaatgctttaagcttagtcataaactacatttgcctatggacctttggttattttgttaatgttgtcattatatttaagacttattgtaggaagtaattttaaatctatttcataacctgttttgatctgtctgcatactgctatatgttaatttattacggcttttacaattcacttgtcatctgagattgttctagaaactgatgaggaaagagtcgtactaaccaaaatgaatgggcaattgacataacattctatttcaggtttccttgtacttggggagccaaagcacaagatctaaggaaatcttattcacatttgtccctcagagcatcttaccctaagccacagaggtataaatagtaagatgagaattgctcattgtctgactccttgcacagcatgaatttaattctagttggaaacaaagttagtgttgactactggttaccttaaagtattcaccatactatatatttattagaccatctttcctctttgtttattcaagactatgtacttataaaagagaacctataagaatgtgtagagtgaccaaatagggacaaagtgacatacctagagtttgctgctcaaatttttccactagcgtaaccctaaagcagaggggcatgatgaagggggaaggggttctgagagcaaaatcagaatcagcctaccactgacatgaagtgtgttctgtctttaaaatgtaagtgaattttgcattctctataataaaacttttaatataaaaatgatatgtttaatctttactgaaacttatgccaccacaaaggagcaccatgttcatcacgtggctgctcagttttttcccccagtgccctcaccgccagtggaggcaagtccttgagctacacacactggctgtagtgcaagatatataaacataggccgggctcggtggctcatgcctgtaatcctagcactttgggaggccgaagcaggtggatcatctgaggtcgggagattgagactagcctgaccgacatggagaaaccctgtgtccactaaaaatacaaaattagccgggtgtggtggcacatgcctgtaatcccagctactcgggaggctgaggcaggagaatcacttgaacccaggaggtggaggttgtggtgagccaagatcgcgccgttgcactccagcctgggcaacaagagtgaaactccgtctcaaaaaaaaaaaaaaaagatatataaacatagaacatatccagactatttcctgcccttggaatctgaccttcagaaggattaacgccaaactaaactgacctgaaacagttttgttactattggcattccttgactaaaaaggaccacctgatgctcagtcccgagagtaggaatcgtggtttattatttttcatatccataccacctagttcagtacctgttatagagtgtttaattgctgatactaggaattaagggattttttaaaagtagcaaatagtttaaggaacaatcaaaaaatgaattttctaattcagttttttcaatgaagtcccatcactgaactttataatgtaatgtttattaagcagacatccccttaaatactcagtttctttggagagagcaaagcatatctccctgtggaaattatatataatgcctggtattttacactcaaagcaaggtgttggccaggaaggtaaagaaatggtgtctatgtagacttaatatcactagtctaagtgtcattttgagcctagtagcactaccttccaagtgagtcacaacaaatttgatcctatttgtaggttttttccactgnnatgattcntcatgtatcttacaagagccantcaagcaagactctgcttctatgtatggtgaggccttgttgttctagggtagaataaactctttgtatgcctcattgaatatgccaggtaaaatatatgcagtcaagaatgaattatttttctgactaaagtgtgtagcagtagttcaaaattgtgcccttgttttaacagtttctgtcaacatcttctcatttttccctacaaaaacaccagggtgtattataagtactgcctgtgagaatttgcactttatgtatttgtgtgtggatttcttgtggttttagccaaatgaagtgttatcagtaataaacaggtctnttcatagg

SEQ ID NO: 38 - protein, 622 aa, Homo sapiens
MAENTEGDLNSNLLHAPYHTGDPQLDTAIGQWLRWDKNPKTKEQIENLLRNGMNKELRDRLCCRMTFGTAGLRSAMGAGFCYINDLTVIQSTQGMYKYLERCFSDFKQRGFVVGYDTRGQVTSSCSSQRLAKLTAAVLLAKDVPVYLFSRYVPTPFVPYAVQKLKAVAGVMITASHNRKEDNGYKVYWETGAQITSPHDKEILKCIEECVEPWNGSWNDNLVDTSPLKRDPLQDICRRYMEDLKKICFYRELNSKTTLKFVHTSFHGVGHDYVQLAFKVFGFKPPIPVPEQKDPDPDFSTVKCPNPEEGESVLELSLRLAEKENARVVLATDPDADRLAAAELQENGCWKVFTGNELAALFGWWMFDCWKKNKSRNADVKNVYMLATTVSSKILKAIALKEGFHFEETLPGFKWIGSRIIDLLENGKEVLFAFEESIGFLCGTSVLDKDGVSAAVVVAEMASYLETMNITLKQQLVKVYEKYGYHISKTSYFLCYEPPTIKSIFERLRNFDSPKEYPKFCGTFAILHVRDVTTGYDSSQPNKKSVLPVSKNSQMITFTFQNGCVATLRTSGTEPKIKYYAEMCASPDQSDTALLEEELKKLIDALIENFLQPSKNGLIWRSV

SEQ ID NO: 39 - DNA, 2015 bp, Homo sapiens
acaaataataaggaacaaggtgtggttattgattctctaaaattaagtgaggagatgaagcccaatctagatggtgttgatttattcaacaatggtggttctggaaacggtgaaacgaaaactgggctgagactgaaagcaataaatctgcctttggaaaatgaagtaactgaaatttcagctttgcaggtgcatttggatgaattccaaaaaatcttatggaaggaaagagaaatgcgcacagctttggaaaaagaaatagagagactggagtcggctttgtctctgtggaagtggaagtatgaagaactgaaagaatcaaagccaaaaaatgtgaaagagtttgacattcttcttggtcaacataatgatgaaatgcaagaactgtcaggcaatataaaggaagaatccaaatctcaaaacagcaaagacagagtgatttgtgagttaagagcagagctagagagattgcaagctgaaaatacctcggagtgggacaagagggaaatacttgaaagagaaaagcagggactggagagagaaaatagaaggctgaagatccaggtgaaagaaatggaagagcttttggataagaaaaatagattaagtgcaaactctcaaagtcctgatttcaagatgtcacaaattgatctgcaagaaaaaaaccaggaattactgaaccttcaacatgcctactataaactaaacagacaataccaggcaaatattgcagaactgactcatgcaaacaaccgagtggatcaaaatgaagcagaagtaaagaaactaagattacgagtggaagaactaaagcagggactcaaccaaaaagaagatgagcttgatgattccctgaatcagatccgtaagctccagaggtctctggatgaagagaaagaaagaaatgaaaacttagagactgaactcaggcacttgcaaaactggtaattttttcacaaaatatgctgaattaaagattagggccttaaagacatttccatatccttttcttaaatatcagtaaaattgtttttattaactagaaatattaatgaaaaaaacgtagacaatacacaaattaatgggcttcttcacttcttctaatttttgcctaacagatactgcatattctcaaaaagacaatttaaatgtcatttaaaaacaactttaattctaagatgtgtaaatattttgaaagtcaaaaagggctttcagaatactttttacataaaatctgaaagagttataatatcggtaagaaaaagtaagttgaaaaccatacaagacgctgggtcattaataagaaaaccattgactttaagtataaagtactggtttgtttaaataattggtaaacttttatgtacgtgttgtctatgtggtggggatggcaggttgtattaacaaaaatgaatcattctagaggtgtaacaatacatttcttatataattttataagtcatttctaatctttgtataaaacagaagtgagcaagatgaatcagaaaaaaggtgttttgtattttaaaggtaacagataaccaggtgattgaatctaagacaggctgtaagcatcgctgagaaactaaaaggacttttgacttttatctggatagacatttctacagtaaaatcatggaaaggcatcagcattgcaaagtagcatctaggtagaaatcaggccaaaattaagctgtggtttccctctgagtagtgggaatagagaaaattaggaaattgtggttatgtgaatatttctttaaaacttttatgtacattatagtttattgcttcatatttaagtttagtttttaaggtaaaatgttattttgaacaaaaagacacttataattttccatacctattttcaactgaaggcaacttgtaagatttaactcagtcaataacatactggttttactcatctccccctccattgattagccaaaaaaaaatgaaatcttactaattcattattgaataaagaccacttttatcagagtcaaaaaaaaaaa

SEQ ID NO: 40 - protein, 297 aa, Homo sapiens
MKPNLDGVDLFNNGGSGNGETKTGLRLKAINLPLENEVTEISALQVHLDEFQKILWKEREMRTALEKEIERLESALSLWKWKYEELKESKPKNVKEFDILLGQHNDEMQELSGNIKEESKSQNSKDRVICELRAELERLQAENTSEWDKREILEREKQGLERENRRLKIQVKEMEELLDKKNRLSANSQSPDFKMSQIDLQEKNQELLNLQHAYYKLNRQYQANIAELTHANNRVDQNEAEVKKLRLRVEELKQGLNQKEDELDDSLNQIRKLQRSLDEEKERNENLETELRHLQNW

SEQ ID NO: 41 - DNA, 2372 bp, Hmo sapiens
cgataatgactttgcacgattcactttgggatctcaaagtgcttccaaagcattcagatttacaaacaattcacaagacaggtcatctttgtaatacccatacttacaacgaattaacaagaggagtgacttaagattctccaggaacacagtggcagctattgatgatctgttttctatctgtttgatagagcatcatgagaaatcacaaaatacaatgctatttttctgatgtgtgctaataaagtcaaagaaagcaaatacatcttgacacttttgtccattttcattaaaaaaaaaaaaaagttcagggtgtttggaattttacacctcagcacaccttactggtatcaatggataaagcgggtaattgacagatccacccaaatgccactgcagtcagaagcagatctggacacacccttgtttacagtttcatattgggttgctatagttcccgtgctaaatcaccagctttcaggaacatgactgctcctggcagtggaaggtgctgaaacagaaattttaattaaaaactttatcaagtactcttcacagtgctgcttggcaccatagaaaatcagtacaatatatcgagccctactttggaggagctggatttctgagggagctgatccagttctaagtgtcttctcgaattaggagatagatgatctttgatggggatctcctccgtcaccacaggccagtcacagaaccaactagccacgtgctgccagacctcagtgggcccaagcaggagcaatctcttctatcccccatctcccccaggaccatcccccccattgtcaatgtcatccagggctcttctggtagtgagtgacttttctgcacatgtttagggcttgggggagctagaacacaggaaacatgaatgcaaaaggcatggaaaacactgttttgctttgggttagtaaaatgtgggcaggacaaagattactattggtcggagctttgccaagtgagatagaatcaactgtcaccccattcctttcccagaaggtcttatggtattaaggatacatccagtattttcccacagatttttattcaggcgatgtttcataaattacatatatgaaaacattcattattacatttccttgtgtgtttcaaacagacattggcaccttcctattgagttaattctctgcatcttttgcagcagcagcccgcaaggagattcccagagatggctcccctaacacacagtcctgtgattttacagttctatgacttacagttgatgattcacaagattcaggattctacaagactcaagggggaactaaactttcttacgattgtacatgatcagttatagggctgtaatcattaattgttggcttcaaatgtggacacacacacacacacatcatgccaaggagggaatggggtgtttcaagtcaggcagcgatgattctggaaggttggaaatgtaaggttagaagcttggctggtcttagtaaacttgttcccttgctcccaccaagaagaggtaccaaatgtgagacctgagatctcctccaatatctgtcctctgcagttccgggaaactaatcatgaagtacacatgcagcagctcctccacttcctttcctccgaggtcctcctttccattctcccacctagatactgacacaccgccacggtttccacattggaagggcagaacactgtgcagtatcgtgcacacttgctgggttaggaatagagctgccctagggtcaccttcatgcaagtattgacagctacaaattaaagtccttagagcagttgacacagatactacgttctagaagagaattaaatttaaacgtcaagtttaaagggatcataattctgcaggtatctttctctgagtgactgaatgtgactattgcattagggtaaatgaattaagacgtgcaagtgggatttactgtatgttagaaaggagttttgcagccaagactgccttgaataaaatgtgtttgcactgaaaaaaaattttaaattacttggtctctggttgctgtaaaggtcatccaagatggatgttctgtttatattgtatagtatttcatatgaaataattacagttcatgaaatgtcttccctaatgttactgatttataacagcacatttgtaacatggtttttatcgtgtcagtgtaccatactgtaaatgatgattacttgtcatgcttagtataataacttaaaagaaaaaaaaggacagggatttttgtaagtctatatttgaaagtccctccctatggtgatactgtgttcatgttgtttatgtagtgttgtgtgaaatatccattttggattgtgttactttttaagatattaaataacatttggtt

SEQ ID NO: 42 - protein, 365 aa, Homo sapiens
KRKVAQEAIESLSASKLAKSICSQFRTRLNSSHEAFAASLRQLEAGHSGRLEKTEDLWLRVRKDHAPRLARLSLESRSLQDVLLHRKPKLGQELGRGQYGVVYLCDNWGGHFPCALKSVVPPDEKHWNDLALEFHYMRSLPKHERLVDLHGSVIDYNYGGGSSIAVLLIMERLHRDLYTGLKAGLTLETRLQIALDVVEGIRFLHSQGLVHRDIKLKNVLLDKQNRAKITDLGFCKPEAMMSGSIVGTPIHMAPELFTGKYDNSVDVYAFGILFWYICSGSVKLPEAFERCASKDHLWNNVRRGARPERLPVFDEECWQLMEACWDGDPLKRPLLGIVQPMLQGIMNRLCKSNSEQPNRGLDDST

SEQ ID NO: 43 - protein, 1185 aa, Homo sapiens
KKIREKFNRYLDVVNRNKQVVEASYTAHLTSPLTAIQDCCTIPPSMMEFDGNFNTNVSRTISCDRLSTTVNSRAFNPGRDLNSVLADNLKSNPGIKWQYFSSEEGIFTVFPAHKFRCKGSYEHRSRPIYVSTVRPQSKHIVVILDHGASVTDTQLQIAKDAAQVILSAIDEHDKISVLTVADTVRTCSLDQCYKTFLSPATSETKRKMSTFVSSVKSSDSPTQHAVGFQKAFQLIRSTNNNTKFQANTDMVIIYLSAGITSKDSSEEDKKATLQVINEENSFLNNSVMILTYALMNDGVTGLKELAFLRDLAEQNSGKYGVPDRTALPVIKGSMMVLNQLSNLETTVGRFYTNLPNRMIDEAVFSLPFSDEMGDGLIMTVSKPCYFGNLLLGIVGVDVNLAYILEDVTYYQDSLASYTFLIDDKGYTLMHPSLTRPYLLSEPPLHTDIIHYENIPKFELVRQNILSLPLGSQIIAVPVNSSLSWHINKLRETGKEAYNVSYAWKMVQDTSFILCIVVIQPEIPVKQLKNLNTVPSSKLLYHRLDLLGQPSACLHFKQLATLESPTIMLSAGSFSSPYEHLSQPETKRMVEHYTAYLSDNTRLIANPGLKFSVRNEVMATSHVTDEWMTQMEMSSLNTYIVRRYIATPNGVLRIYPGSLMDKAFDPTRRQWYLHAVANPGLISLTGPYLDVGGAGYVVTISHTIHSSSTQLSSGHTVAVMGIDFTLRYFYKVLMDLLPVCNQDGGNKIRCFIMEDRGYLVAHPTLIDPKGHAPVEQQHITHKEPLVANDILNHPNFVKKNLCNSFSDRTVQRFYKFNTSLAGDLTNLVHGSHCSKYRLARIPGTNAFVGIVNETCDSLAFCACSMVDRLCLNCHRMEQNECECPCECPLEVNECTGNLTNAENRNPSCEVHQEPVTYTAIDPGLQDALHQCVNSRCSQRLESGDCFGVLDCEWCMVDSDGKTHLDKPYCAPQKECFGGIVGAKSPYVDDMGAIGDEVITLNMIKSAPVGPVAGGIMGCIMVLVLAVYAYRHQIHRRSHQHMSPLAAQEMSVRMSNLENDRDERDDDSHEDRGIISNTRFIAAVIERHAHSPERRRRYWGRSGTESDHGYSTMSPQEDSENPPCNNDPLSAGVDVGNHDEDLDLDTPPQTAALLSHKFHHYRSHHPTLHHSHHLQAAVTVHTVDAEC

SEQ ID NO: 44 - protein, 410 aa, Homo sapiens
MDEVSLLSELVEAASFLQVTSLLQLLLSQVRLNNCLEMYRLAQVYGLPDLQEACLRFMVVHFHEVLCKPQFHLLGSPPQAPGDVSLKQRLREARMTGTPVLVALGDFLGGPLAPHPYQGEPPSMLRYEEMTERWFPLANNLPPDLVNVRGYGSAILDNYLFIVGGYRITSQEISAAHSYNPSTNEWLQVASMNQKRSNFKLVAVNSKLYAIGGQAVSNVECYNPEQDAWNFVAPLPNPLAEFSACECKGKIYVIGGYTTRDRNMNILQYCPSSDMWTLFETCDVHIRKQQMVSVEETIYIVGGCLHELGPNRRSSQSEDMLTVQSYNTVTRQWLYLKENTSKSGLNLTCALHNDGIYIMSRDVTLSTSLEHRVFLKYNIFSDSWEAFRRFPAFGHNLLVSSLYLPNKAET

SEQ ID NO: 45 - protein, 1101 aa, Homo sapiens
RRRRPSSSRRLRGRGAAQMACPALGLEALQPLQPEPPPEPAFSEAQKWIEQVTGRSFGDKDFRTGLENGILLCELLNAIKPGLVKKINRLPTPIAGLDNIILFLRGCKELGLKESQLFDPSDLQDTSNRVTVKSLDYSRKLKNVLVTIYWLGKAANSCTSYSGTTLNLKEFEGLLAQMRKDTDDIESPKRSIRDSGYIDCWDSERSDSLSPPRHGRDDSFDSLDSFGSRSRQTPSPDVVLRGSSDGRGSDSESDLPHRKLPDVKKDDMSARRTSHGEPKSAVPFNQYLPNKSNQTAYVPAPLRKKKAEREEYRKSWSTATSPLGGERPFRYGPRTPVSDDAESTSMFDMRCEEEAAVQPHSRARQEQLQLINNQLREEDDKWQDDLARWKSRRRSVSQDLIKKEEERKKMEKLLAGEDGTSERRKSIKTYREIVQEKERRERELHEAYKNARSQEEAEGILQQYIERFTISEAVLERLEMPKILERSHSTEPNLSSFLNDPNPMKYLRQQSLPPPKFTATVETTIARASVLDTSMSAGSGSPSKTVTPKAVPMLTPKPYSQPKNSQDVLKTFKVDGKVSVNGETVHREEEKERECPTVAPAHSLTKSQMFEGVARVHGSPLELKQDNGSIEINIKKPNSVPQELAATTEKTEPNSQEDKNDGGKSRKGNIELASSEPQHFTTTVTRCSPTVAFVEFPSSPQLKNDVSEEKDQKKPENEMSGKVELVLSQKVVKPKSPEPEATLTFPFLDKMPEANQLHLPNLNSQVDSPSSEKSPVTTPQFKFWAWDPEEERRRQEKWQQEQERLLQERYQKEQDKLKEEWEKAQKEVEEEERRYYEEERKIIEDTVVPFTVSSSSADQLSTSSSMTEGSGTMNKIDLGNCQDEKQDRRWKKSFQGDDSDLLLKTRESDRLEEKGSLTEGALAHSGNPVSKGVHEDHQLDTEAGAPHCGTNPQLAQDPSQNQQTSNPTHSSEDVKPKTLPLDKSINHQIESPSERRKKSPREHFQAGPFSPCSPTPPGQSPNRSISGKKLCSSCGLPLGKGAAMIIETLNLYFHIQCFRCGICKGQLGDAVSGTDVRIRNGLLNCNDCYMRSRSAGQPTTL

SEQ ID NO: 46 - protein, 1161 aa, Homo sapiens
MKKFSRMPKSEGGSGGGAAGGGAGGAGAGAGCGSGGSSVGVRVFAVGRHQVTLEESLAEGGFSTVFLVRTHGGIRCALKRMYVNNMPDLNVCKREITIMKELSGHKNIVGYLDCAVNSISDNVWEVLILMEYCRAGQVVNQMNKKLQTGFTEPEVLQIFCDTCEAVARLHQCKTPIIHRDLKVENILLNDGGNYVLCDFGSATNKFLNPQKDGVNVVEEEIKKYTTLSYRAPEMINLYGGKPITTKADIWALGCLLYKLCFFTLPFGESQVAICDGNFTIPDNSRYSRNIHCLIRFMLEPDPEHRPDIFQVSYFAFKFAKKDCPVSNINNSSIPSALPEPMTASEAAARKSQIKARITDTIGPTETSIAPRQRPKANSATTATPSVLTIQSSATPVKVLAPGEFGNHRPKGALRPGNGPEILLGQGPPQQPPQQHRVLQQLQQGDWRLQQLHLQHRHPHQQQQQQQQQQQQQQQQQQQQQQQQQQQHHHHHHHHLLQDAYMQQYQHATQQQQMLQQQFLMHSVYQPQPSASQYPTMMPQYQQAFFQQQMLAQHQPSQQQASPEYLTSPQEFSPALVSYTSSLPAQVGTIMDSSYSANRSVADKEAIANFTNQKNISNPPDMSGWNPFGEDNFSKLTEEELLDREFDLLRSNRLEERASSDKNVDSLSAPHNHPPEDPFGSVPFISHSGSPEKKAEHSSINQENGTANPIKNGKTSPASKDQRTGKKTSVQGQVQKGNDESESDFESDPPSPKSSEEEEQDDEEVLQGEQGDFNDDDTEPENLGHRPLLMDSEDEEEEEKHSSDSDYEQAKAKYSDMSSVYRDRSGSGPTQDLNTILLTSAQLSSDVAVETPKQEFDVFGAVPFFAVRAQQPQQEKNEKNLPQHRFPAAGLEQEEFDVFTKAPFSKKVNVQECHAVGPEAHTIPGYPKSVDVFGSTPFQPFLTSTSKSESNEDLFGLVPFDEITGSQQQKVKQRSLQKLSSRQRRTKQDMSKSNGKRHHGTPTSTKKTLKPTYRTPERARRHKKVGRRDSQSSNEFLTISDSKENISVALTDGKDRGNVLQPEESLLDPFGAKPFHSPDLSWHPPHQGLSDIRADHNTVLPGRPRQNSLHGSFHSADVLKMDDFGAVPFTELVVQSITPHQSQQSQPVELDPFGAAPFPSKQ

(19) United States
(12) Patent Application Publication
(10) Pub. No.: US 2007/0110744 A1
(43) Pub. Date: May 17, 2007

(54) Lymphatic and blood endothelial cell genes

(76) Inventors: Kari Alitalo, Helsinki (FI); Taija Makinen, Martinsried (DE); Tatiana Petrova, Helsinki (FI); Pipsa Saharinen, Helsinki (FI); Juha Saharinen, Helsinki (FI)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/570,963

(22) PCT Filed: Sep. 8, 2004
(86) PCT No.: PCT/US04/29245

§ 371 Date: Oct. 2, 2006

Related U.S. Application Data
(60) Provisional application No. 60/500,977, filed on Sep. 8, 2003.

(51) Int. Cl. A61K 48/00; A61K 38/17; A61K 39/395; C12N 5/08

(52) U.S. Cl. 424/143.1; 514/12; 514/44; 435/366; 435/455

(57) ABSTRACT

The invention provides polynucleotides and genes that are differentially expressed in lymphatic versus blood vascular endothelial cells. These genes are useful targets for treating diseases involving lymphatic vessels, such as lymphedeman, various inflammatory diseases, and cancer metastasis via the lymphatic system.

### BACKGROUND OF THE INVENTION

1. Field of the Invention

The invention relates to polynucleotides and proteins specifically expressed in lymphatic endothelial cells.

2. Description of the Related Art

Recent evidence on the association of lymphangiogenic growth factors with intralymphatic growth and metastasis of cancers (Mandriota, et al., EMBO J. 20:672-682 (2001); Skobe, et al., Nat. Med. 7:192-198 (2001); Stacker, et al., Nat. Med. 7:186-191 (2001); Karpanen, et al., Cancer Res. 61:1786-1790 (2001)) has raised hopes that lymphatic vessels could be used as an additional target for tumor therapy. Cancer cells spread within the body by direct invasion to surrounding tissues, spreading to body cavities, invasion into the blood vascular system (hematogenous metastasis), as well as spread via the lymphatic system (lymphatic metastasis). Regional lymph node dissemination is the first step in the metastasis of several common cancers and correlates highly with the prognosis of the disease. The lymph nodes that are involved in draining tissue fluid from the tumor area are called sentinel nodes, and diagnostic measures are in place to find these nodes and to remove them in cases of suspected metastasis. However, in spite of its clinical relevance, little is known about the mechanisms leading to metastasis via the bloodstream or via the lymphatics.

Until recently, the lymphatic vessels have received much less attention than blood vessels, despite their importance in medicine. Lymphatic vessels collect protein-rich fluid and white blood cells from the interstitial space of most tissues and transport them as a whitish opaque fluid, the lymph, into the blood circulation. Small lymphatic vessels coalesce into larger vessels, which drain the lymph through the thoracic duct into large veins in the neck region. Lymph nodes serve as filtering stations along the lymphatic vessels and lymph movement is propelled by the contraction of smooth muscles surrounding collecting lymphatic vessels and by bodily movements, the direction of flow being secured by valves as it is in veins. The lymphatic capillaries are lined by endothelial cells, which have distinct junctions with frequent large interendothelial gaps. The lymphatic capillaries also lack a continuous basement membrane, and are devoid of pericytes. Anchoring filaments connect the abluminal surfaces of lymphatic endothelial cells to the perivascular extracellular matrix and pull to maintain vessel patency in the presence of tissue edema. The absence or obstruction of lymphatic vessels, which is usually the result of an infection, surgery, or radiotherapy and in rare cases, a genetic defect, causes accumulation of a protein-rich fluid in tissues, lymphedema The lymphatic system is also critical in fat absorption from the gut and in immune responses. Bacteria, viruses, and other foreign materials are taken up by the lymphatic vessels and transported to the lymph nodes, where the foreign material is presented to immune cells and where dendritic cells traverse via the lymphatics. There has been slow progress in the understanding of and ability to manipulate the lymphatic vessels.

Abnormal development or function of the lymphatic ECs can result in tumors or malformations of the lymphatic vessels, such as lymphangiomas or lymphangiectasis. Witte, et al., Regulation of Angiogenesis (eds. Goldber, I. D. & Rosen, E. M.) 65-112 (Birkäuser, Basel, Switzerland, 1997). The VEGFR-3 tyrosine kinase receptor is expressed in the normal lymphatic endothelium and is upregulated in many types of vascular tumors, including Kaposi's sarcomas. Jussila, et al., Cancer Res 58, 1955-1604 (1998); Partanen, et al., Cancer 86:2406-2412 (1999). Absence or dysfunction of lymphatic vessels which can result from an infection, surgery, radiotherapy or from a genetic defect, causes lymphedema, which is characterized by a chronic accumulation of protein-rich fluid in the tissues that leads to swelling. The importance of VEGFR-3 signaling for lymphangiogenesis was revealed in the genetics of familial lymphedema, a disease characterized by a hypoplasia of cutaneous lymphatic vessels, which leads to a disfiguring and disabling swelling of the extremities. Witte, et al., Regulation of Angiogenesis (eds. Goldber, I. D. & Rosen, E. M.) 65-112 (Birkäuser, Basel, Switzerland, 1997); Rockson, S. G., Am. J. Med. 110, 288-295 (2001). Some members of families with lymphedema are heterozygous for missense mutations of the VEGFR3 exons encoding the tyrosine kinase domain, which results in an inactive receptor protein. Karkkainen, et al., Nature Genet. 25:153-159 (2000); Irrthum, et al., Am. J. Hum. Genet. 67:295-301 (2000).

There is a need in the art for information on the transcriptional program which controls the diversity of endothelial cells, and into the mechanisms of angiogenesis and lymphangiogenesis. There is also a need in the art for new vascular markers, which may be used as valuable targets in the study of a number of diseases involving the lymphatic vessels, including tumor metastasis.

### SUMMARY OF THE INVENTION

The compositions of the present invention include isolated polynucleotides and polypeptides, in particular, lymphatic endothelial genes, isolated polypeptides encoded by these polynucleotides, recombinant DNA molecules, cloned genes or degenerate variants thereof, especially naturally occurring variants such as allelic variants, and antibodies that specifically recognize one or more epitopes present on such polypeptides.

The compositions of the present invention additionally include vectors, including expression vectors, containing the polynucleotides of the invention, cells genetically engineered to contain such polynucleotides and cells genetically engineered to express such polynucleotides.

In selected embodiments, such isolated polynucleotides of the invention represent a polynucleotide comprising a nucleotide sequence selected from the group consisting of SEQ ID NOS: 5, 7, 9, 11, 13, 15, 17, 19, 21, 23, 25, 27, 29, 31, 33, 35, 37, 39 and 41, or a fragment thereof that encodes a corresponding polypeptide.

The polynucleotides of the present invention also include, but are not limited to, a polynucleotide that hybridizes to the complement of the nucleotide sequence of SEQ ID NOS: 5, 7, 9, 11, 13, 15, 17, 19, 21, 23, 25, 27, 29, 31, 33, 35, 37, 39 and 41 under highly stringent hybridization conditions; a polynucleotide that hybridizes to the complement of the nucleotide sequence of SEQ ID NOS: 5, 7, 9, 11, 13, 15, 17, 19, 21, 23, 25, 27, 29, 31, 33, 35, 37, 39 and 41 under moderately stringent hybridization conditions; a polynucleotide which is an allelic variant of any polynucleotide recited above; a polynucleotide which encodes a species homologue of any of the proteins recited above; or a polynucleotide that encodes a polypeptide comprising a specific domain or truncation of the polypeptide encoded by any one of SEQ ID NOS: 5, 7, 9, 11, 13, 15, 17, 19, 21, 23, 25, 27, 29, 31, 33, 35, 37, 39 and 41. Exemplary high stringency hybridization conditions are hybridization at 42° C. for 20 hours in a solution containing 50% formamide, 5×SSPE, 5× Denhardt's solution, 0.1% SDS and 0.1 mg/ml denatured salmon sperm DNA, with a wash in 1×SSC, 0.1% SDS for 30 minutes at 65° C.

Another aspect of the invention is drawn to LEC and BEC polypeptides, including polypeptides encoded by the polynucleotides described above. In some embodiments, the polypeptides are the mature forms of the polypeptides of the invention. Expressly contemplated is a purified and isolated polypeptide comprising an amino acid sequence selected from the group consisting of SEQ ID NOS: 6, 8, 10, 12, 14, 16, 18, 20, 22, 24, 26, 28, 30, 32, 34, 36, 38, 40, 42, 43, 44, 45, and 46; and a purified and isolated polypeptide comprising an amino acid sequence selected from the group consisting of: (a) SEQ ID NOS: 6, 8, 10, 12, 14, 16, 20, 22, 24, 42 and 43; and (b) an extracellular domain fragment of at least 10 amino acids of an amino acid sequence of (a). Further, this aspect of the invention includes a purified and isolated, soluble polypeptide as described immediately above, comprising an extracellular domain fragment of an amino acid sequence selected from the group consisting of: SEQ ID NOS: 6, 8, 10, 12, 14, 16, 20, 22, 24, 42 and 43 wherein the polypeptide lacks any transmembrane domain. Such a polypeptide may further lack any intracellular domain. Also, the invention contemplates a fusion protein comprising a polypeptide as described above fused, e.g., to an immunoglobulin fragment comprising an immunoglobulin constant region.

Another aspect of the invention is an isolated polypeptide comprising an amino acid sequence at least 95% identical to amino acids 1-1005 of SEQ ID NO: 43. In a related aspect, a composition is contemplated which comprises a soluble polypeptide comprising a fragment of one of the amino acid sequences disclosed herein that comprises a functional protein domain. One exemplary embodiment comprises a fragment of the amino acid sequence set forth in SEQ ID NO: 43, wherein said fragment lacks the transmembrane and intracellular amino acids from approximately residues 1006-1028 of SEQ ID NO: 43. Further contemplated is an isolated polypeptide comprising an adhesion domain having an amino acid sequence at least 95% identical to a fragment of a polypeptide comprising the amino acid sequence set forth in SEQ ID NO: 20 or SEQ ID NO: 43, wherein said fragment includes at least one vonWillebrand factor domain or at least one Cache domain.

In an additional aspect, the invention contemplates polypeptide fragments of any one of SEQ ID NOS: 6, 8, 10, 12, 14, 16, 18, 20, 22, 24, 26, 28, 30, 32, 34, 36, 38, 40, 42, 43, 44, 45, and 46, wherein the fragment comprises at least one protein domain of the given polypeptide sequence, as set out herein. The invention further contemplates polypeptide fragments comprising an amino acid sequence at least 95% identical to a fragment of any one of SEQ ID NOS: 6, 8, 10, 12, 14, 16, 18, 20, 22, 24, 26, 28, 30, 32, 34, 36, 38, 40, 42, 43, 44, 45, and 46 comprising a protein domain of the polypeptide set forth herein. Exemplary embodiments include, e.g., polypeptide fragments comprising a protein domain that is a protein kinase domain selected from the group consisting of a protein kinase domain set out in SEQ ID NO: 8, a protein kinase domain set out in SEQ ID NO: 26, a protein kinase domain at approximately amino acids 88-342 of SEQ ID NO: 42, and a protein kinase domain at approximately amino acids 51-317 of SEQ ID NO: 46; a Zinc binding domain selected from the group consisting of a Zinc-binding domain set out in SEQ ID NO: 36, a zinc-binding domain at approximately amino acids 627-561 of SEQ ID NO: 6 and a zinc-binding domain at approximately amino acids 1030-1094 of SEQ ID NO: 45.

Another aspect of the invention is drawn to an isolated polypeptide at least 95% identical to a polypeptide set forth herein, comprising a particular biological activity associated with a protein domain as set forth herein. Exemplary biological activities include ubitquitin ligase activity of a Hect domain in SEQ ID NO: 28; guanine-nucleotide-dissociation-stimulating activity of at least one RCC1-like domain in SEQ ID NO: 28; cation transport activity of a cation transport domain set forth in SEQ ID NO: 12; GTP-binding activity of at least one GTP-binding domain set forth in SEQ ID NO: 32, SRPY domain activity of at least one SPRY domain of SEQ ID NO: 6, and Kelch domain activity of at least one Kelch domain of SEQ ID NO: 30 or SEQ ID NO: 43. In another aspect, the invention contemplates an isolated polypeptide comprising an amino acid sequence at least 95% identical to a transcription factor selected from the group consisting of SEQ ID NO: 30, SEQ ID NO: 38, SEQ ID NO: 40, and SEQ ID NO: 44, or a protein involved in cytoskeletal regulation selected from the group consisting of SEQ ID NO: 18, SEQ ID NO: 36 and SEQ ID NO: 45.

In a related aspect, the invention provides a composition comprising a polynucleotide or polypeptide or protein as described above and a pharmaceutically acceptable diluent, carrier or adjuvant. Polypeptide compositions of the invention may comprise an acceptable carrier, such as a hydrophilic, e.g., pharmaceutically acceptable, carrier. Further provided is a kit comprising such a composition and a protocol for administering the pharmaceutical composition to a mammalian subject to modulate the lymphatic system in the subject. The invention also provides an antibody that specifically binds to a polypeptide as described above, and that antibody is humanized in some embodiments. Antibodies contemplated by the invention include, for example, monoclonal and polyclonal antibodies, single chain antibodies, chimeric antibodies, bifunctional/bispecific antibodies, humanized antibodies, human antibodies, single and multiple domain antibodies and complementarity-determining region (CDR)-grafted antibodies, including compounds which include CDR sequences that specifically recognize a polypeptide of the invention, provided that such antibodies each specifically bind at least one polypeptide according to the invention. Still further, the invention provides a protein comprising an antigen binding domain of an antibody that specifically binds to a polypeptide as described hereinabove, wherein the protein specifically binds to the polypeptide.

The invention also relates to methods for producing a polypeptide comprising growing a culture of the cells of the invention in a suitable culture medium, and purifying the protein from the culture or from an extract of the cells. In particular, the invention contemplates a method for producing a LEC polypeptide comprising steps of growing a host cell transformed or transfected with an expression vector as described herein under conditions in which the cell expresses the polypeptide encoded by the polynucleotide.

Methods of identifying the products and compositions described herein are also provided by the invention. In particular, the invention provides a method of identifying a LEC nucleic acid comprising: (a) contacting a biological sample containing a candidate LEC nucleic acid with a polynucleotide comprising a fragment of at least 14 contiguous nucleotides of a sequence selected from the group consisting of SEQ ID NOS: 5, 7, 9, 11, 13, 15, 17, 19, 21, 23, 25, 27, 29, 31, 33, 35, 37, 39 and 41, or a complement thereof, under the following stringent hybridization conditions:

(i) hybridization at 42° C. for 20 hours in a solution containing 50% formamide, 5×SSPE, 5× Denhardt's solution, 0.1% SDS and 0.1 mg/ml denatured salmon sperm DNA, and (ii) washing for 30 minutes at 65° C. in 1×SSC, 0.1% SDS; and (b) detecting hybridization of the candidate LEC nucleic acid and the polynucleotide, thereby identifying a LEC nucleic acid.

The invention also provides a method of identifying a LEC protein comprising: (a) contacting a biological sample containing a candidate LEC protein with a LEC protein binding partner selected from the group consisting of an antibody as described herein or a protein or polypeptide as described herein, under conditions suitable for binding therebetween; and (b) detecting binding between the candidate LEC protein and the LEC binding partner, thereby identifying a LEC protein.

Another related aspect of the invention is a method of identifying a LEC comprising: (a) contacting a biological sample comprising cells with a LEC binding partner under conditions suitable for binding therebetween, wherein the LEC binding partner comprises an antibody that binds to a polypeptide comprising a sequence selected from the group consisting of SEQ ID NOS: 6, 8, 10, 12, 14, 16, 20, 22, 24, 42 and 43, or comprises an antigen binding fragment of the antibody; and (b) identifying a LEC by detecting binding between a cell and the LEC binding partner, where binding of the LEC binding partner to the cell identifies a LEC.

Polynucleotides according to the invention have numerous applications in a variety of techniques known to those skilled in the art of molecular biology. These techniques include use as hybridization probes, use as primers for PCR, use for chromosome and gene mapping, use in the recombinant production of protein, and use in generation of anti-sense DNA or RNA, their chemical analogs and the like. For example, when the expression of an mRNA is largely restricted to a particular cell or tissue type, such as a lymphatic endothelial cell, polynucleotides of the invention can be used as hybridization probes to detect the presence of the particular cell or tissue mRNA in a sample using, e.g., in situ hybridization.

In another aspect, the invention provides a composition comprising an isolated polynucleotide that comprises a nucleotide sequence that encodes a polypeptide comprising an amino acid sequence selected from the group consisting of SEQ ID NOs: 6, 8, 10, 12, 14, 16, 18, 20, 22, 24, 26, 28, 30, 32, 34, 36, 38, 40, 42, 43, 44, 45, and 46 and a pharmaceutically acceptable diluent, carrier or adjuvant. In some embodiments, the composition comprises a polynucleotide that comprises a nucleotide sequence selected from the group consisting of SEQ ID NOS: 5, 7, 9, 11, 13, 15, 17, 19, 21, 23, 25, 27, 29, 31, 33, 35, 37, 39 and 41, or a fragment thereof that encodes the polypeptide.

Still another aspect of the invention is an expression vector comprising an expression control sequence operably linked to a polynucleotide that comprises a nucleotide sequence that encodes a polypeptide comprising an amino acid sequence selected from the group consisting of SEQ ID NOs: 6, 8, 10, 12, 14, 16, 18, 20, 22, 24, 26, 28, 30, 32, 34, 36, 38, 40, 42, 43, 44, 45, and 46. In some embodiments, the expression vector is a replication-deficient adenoviral or adeno-associated viral vector containing the polynucleotide. A related aspect of the invention is a composition comprising an expression vector as described above and a pharmaceutically acceptable diluent, carrier, or adjuvant. Further, the invention provides a kit comprising the composition containing either the above-described polynucleotide or vector and a pharmaceutically acceptable diluent, carrier or adjuvant, packaged with a protocol for administering the composition to a mammalian subject to modulate the lymphatic system in the subject.

The invention further provides a host cell transformed or transfected with an expression vector as described above.

The polypeptides according to the invention can be used in a variety of conventional procedures and methods that are currently applied to other proteins. In addition, a polypeptide of the invention can be used to generate an antibody that specifically binds the polypeptide.

In one aspect of the invention, a method is provided for differentially modulating the growth or differentiation of blood endothelial cells (BEC) or lymphatic endothelial cells (LEC), comprising contacting endothelial cells with a composition comprising an agent that differentially modulates blood or lymphatic endothelial cells, said agent selected from the group consisting of: (a) a polypeptide that comprises an amino acid sequence of a BEC polypeptide or a LEC polypeptide, or an active fragment of the polypeptide; (b) a polynucleotide that comprises a nucleotide sequence that encodes a polypeptide according to (a); (c) an antibody that specifically binds to a polypeptide according to (a); (d) a polypeptide comprising a fragment of the antibody of (c), wherein the fragment and the antibody bind to the polypeptide; (e) an antisense nucleic acid to a human gene or mRNA encoding the polypeptide of (a); (f) an interfering RNA (RNAi) to a human gene or mRNA encoding the polypeptide of (a). In a related aspect, the method of the invention provides an antibody, a peptide or a small molecular weight compound for differentially modulating the growth or differentiation of blood endothelial cells (BEC) or lymphatic endothelial cells (LEC). The method may involve endothelial cell contact with the composition ex vivo or in vivo. The composition may comprise a pharmaceutically acceptable diluent, adjuvant, or carrier, and the contacting step may comprise administering the composition to a mammalian subject to differentially modulate BECs or LECs in the mammalian subject.

Further, the method may comprise identifying a human subject with a disorder characterized by hyperproliferation of LECs; and administering to the human subject the composition, wherein the agent differentially inhibits LEC growth compared to BEC growth; alternatively the method may comprise identifying a human subject with a disorder characterized by hyperproliferation of LECs; screening LECs of the subject to identify overexpression of a polypeptide set forth in any of Tables 1 and 2 herein; and administering to the human subject the composition, wherein the agent differentially inhibits LEC growth compared to BEC growth by inhibiting expression of the polypeptide identified by the screening step.

This aspect of the invention also contemplates a method of modulating the growth of lymphatic endothelial cells in a human subject, comprising steps of identifying a human subject with a hypoproliferative lymphatic disorder; screening the subject to identify underexpression or underactivity of a LEC polypeptide set forth in any of Table 1 or Table 2 herein; administering to the human subject the composition, wherein the agent comprises the LEC polypeptide (a) identified by the screening step or an active fragment of the polypeptide, or comprises the polynucleotide (b) that comprises a nucleotide sequence that encodes the polypeptide.

A related aspect of the invention is drawn to a use of an agent for the manufacture of a medicament for the differential modulation of blood vessel endothelial cell (BEC) or lymphatic vessel endothelial cell (LEC) growth or differentiation, the agent selected from the group consisting of: (a) a polypeptide that comprises an amino acid sequence of a BEC polypeptide or a LEC polypeptide, or an active fragment of the polypeptide; (b) a polynucleotide that comprises a nucleotide sequence that encodes a polypeptide according to (a); (c) an antibody that specifically binds to a polypeptide according to (a); (d) a polypeptide comprising a fragment of the antibody of (c), wherein the fragment and the antibody bind to the polypeptide; (e) an antisense nucleic acid to a human gene or mRNA encoding the polypeptide of (a); (f) an interfering RNA (RNAi) to a human gene or mRNA encoding the polypeptide of (a). It is further contemplated that an antibody, a peptide or a small molecular weight compound may be useful for the manufacture of a medicament for the differential modulation of blood endothelial cell (BEC) or lymphatic endothelial cell (LEC) growth or differentiation. In some embodiments, the medicament may promote growth and/or differentiation of a BEC or LEC; in other embodiments, the medicament inhibits growth and/or differentiation of such a cell.

In another aspect, the invention provides a method of identifying compounds that modulate growth of endothelial cells, comprising culturing endothelial cells in the presence and absence of a compound; and measuring expression of at least one BEC or LEC gene in the cells, wherein the gene is a gene set forth in any of Tables 1 and 2 herein, wherein a change in expression of at least one BEC gene in the presence compared to the absence of the compound identifies the compound as a modulator of BEC growth, and wherein a change in expression of at least one LEC gene in the presence compared to the absence of the compound identifies the compound as a modulator of LEC growth. The method may be used to screen for a compound that selectively modulates BEC or LEC growth or differentiation, wherein the measuring step comprises measuring expression of at least one BEC gene and at least one LEC gene in the cells, and wherein the method comprises screening for a compound that selectively modulates BEC or LEC growth or differentiation by selecting a compound that differentially modulates expression of the at least one BEC gene compared to expression of the at least one LEC gene. In a related aspect, the method further comprises a second BEC or LEC gene that encodes a polypeptide set forth in any of Tables 3 and 4 of PCT/US03/06900.

Further, the invention comprehends a method or use according to the aspects of the invention described above, wherein the polypeptide is a LEC polypeptide selected from a gene found in any of Tables 1 and 2 herein, and the agent differentially modulates LEC growth or differentiation over BEC growth or differentiation. In some embodiments, the LEC polypeptide comprises an amino acid sequence selected from the group consisting of SEQ ID NOs: 6, 8, 10, 12, 14, 16, 20, 22, 24, 42 and 43; in other embodiments, the LEC polypeptide comprises an amino acid sequence selected from the group consisting of SEQ ID NO: 20 and SEQ ID NO: 32. In these embodiments, an agent may be an antibody that specifically binds to a LEC polypeptide as described above, or a polypeptide fragment of such an antibody. Further, the agent may be an extracellular domain of a polypeptide described above, a polynucleotide encoding an extracellular domain, or an antisense molecule or nucleic acid. Alternatively, the polypeptide is a BEC polypeptide selected from the BEC polypeptides set forth in any of Tables 1 and 2 herein, and the agent differentially modulates BEC growth or differentiation over LEC growth or differentiation. It is further contemplated that the above methods further comprise screening LECs of the subject to identify overexpression of a second polypeptide set forth in any of Tables 3 and 4 of PCT/US03/06900 (specifically incorporated herein by reference).

The methods of the present invention further relate to methods for detecting the presence of the polynucleotides or polypeptides of the invention in a sample. Such methods can, for example, be utilized as part of a prognostic and/or diagnostic evaluation of disorders as recited above and for the identification of subjects exhibiting a predisposition to such conditions. Furthermore, the invention provides methods for evaluating the efficacy of drugs, and monitoring the progress of patients, involved in clinical trials for the treatment of disorders related to lymphatic endothelial cells.

The invention also provides methods for the identification of compounds that modulate the expression of the polynucleotides and/or polypeptides of the invention. Such methods can be utilized, for example, for the identification of compounds that can ameliorate symptoms of disorders related to expression of proteins encoded by any one of SEQ ID NOS: 5, 7, 9, 11, 13, 15, 17, 19, 21, 23, 25, 27, 29, 31, 33, 35, 37, 39 and 41 as recited above. Such methods can include, but are not limited to, assays for identifying compounds and other substances that interact with (e.g., bind to) the polypeptides of the invention.

Further, the invention provides a method of assaying for risk of developing hereditary lymphedema, comprising (a) assaying nucleic acid of a human subject for a mutation that correlates with a hereditary lymphedema phenotype and alters the encoded amino acid sequence of at least one gene allele of the human subject when compared to the amino acid sequence of the polypeptide encoded by a corresponding wild-type gene allele, wherein the wild-type polypeptide is a polypeptide identified in any of Tables 1 and 2 herein. Alternatively, a method of assaying for risk of developing hereditary lymphedema, comprises (a) assaying nucleic acid of a human subject for a mutation that correlates with a hereditary lymphedema phenotype and alters the encoded amino acid sequence of at least one gene allele of the human subject when compared to the amino acid sequence of the polypeptide encoded by a corresponding wild-type gene allele, wherein the wild-type polypeptide comprises an amino acid sequence selected from the group consisting of SEQ ID NOS: 6, 8, 10, 12, 14, 16, 20, 22, 24, 42 and 43; (b) correlating the presence or absence of the mutation in the nucleic acid to a risk of developing hereditary lymphedema, wherein the presence of the mutation in the nucleic acid correlates with an increased risk of developing hereditary lymphedema, and wherein the absence of the mutation in the nucleic acid correlates with no increased risk of developing hereditary lymphedema.

In another method of assaying for risk of developing hereditary lymphedema, the steps comprise (a) assaying nucleic acid of a human subject for a mutation that alters the encoded amino acid sequence of at least one intracellular protein or transcription factor allele of the human subject and alters transcription modulation activity of the transcription factor polypeptide encoded by the allele, when compared to the transcription modulation activity of a transcription factor polypeptide encoded by a wild-type allele, wherein the wild-type intracellular protein or transcription factor polypeptide comprises an amino acid sequence selected from the group consisting of SEQ ID NO: 18, 26, 28, 30, 32, 34, 36, 38, 40, 44, 45, and 46; and

(b) correlating the presence or absence of the mutation in the nucleic acid to a risk of developing hereditary lymphedema, wherein the presence of the mutation in the nucleic acid correlates with an increased risk of developing hereditary lymphedema, and wherein the absence of the mutation in the nucleic acid correlates with no increased risk of developing hereditary lymphedema. As one example, the assaying identifies a mutation altering activity of a transactivating or DNA binding domain of a Sox18 allele. In some embodiments of the method, the mutation reduces transcriptional activation of a SOX18-responsive gene compared to transcriptional activation of the gene in the absence of said mutation.

In a related aspect, the invention provides a method of assaying for risk of developing hereditary lymphedema, comprising (a) assaying nucleic acid of a human subject for a mutation that alters the encoded amino acid sequence of at least one LEC gene allele of the human subject and alters the binding affinity of the adhesion polypeptide encoded by the LEC gene allele, when compared to the binding affinity of an adhesion polypeptide encoded by a wild-type allele, wherein the wild-type adhesion polypeptide comprises an amino acid sequence of SEQ ID NO: 20; and (b) correlating the presence or absence of the mutation in the nucleic acid to a risk of developing hereditary lymphedema, wherein the presence of the mutation in the nucleic acid correlates with an increased risk of developing hereditary lymphedema, and wherein the absence of the mutation in the nucleic acid correlates with no increased risk of developing hereditary lymphedema.

In the methods of assaying for risk of developing hereditary lymphedema according to the invention, the assaying may identify the presence of the mutation, and the correlating step may identify the increased risk of the patient developing hereditary lymphedema.

A related method according to the invention is a method of screening a human subject for an increased risk of developing hereditary lymphedema comprising assaying nucleic acid of a human subject for a mutation that alters the encoded amino acid sequence of at least one polypeptide comprising an amino acid sequence found in any of Tables 1 and 2. In some embodiments of this method, the polypeptide comprises an amino acid sequence selected from the group consisting of SEQ ID NOS: 6, 8, 10, 12, 14, 16, 18, 20, 22, 24, 26, 28, 30, 32, 34, 36, 38, 40, 42, 43, 44, 45, and 46 in a manner that correlates with the risk of developing hereditary lymphedema, and it is expressly contemplated that the polypeptide may comprise the amino acid sequences set forth in SEQ ID NOS: 6, 8, 10, 12, 14, 16, 20, 22, 24, 42 and 43.

A related aspect of the invention is drawn to methods of assaying or screening for risk of developing hereditary lymphedema as described above, wherein the method comprises at least one procedure selected from the group consisting of: (a) determining a nucleotide sequence of at least one codon of at least one polynucleotide of the human subject; (b) performing a hybridization assay to determine whether nucleic acid from the human subject has a nucleotide sequence identical to or different from one or more reference sequences; (c) performing a polynucleotide migration assay to determine whether nucleic acid from the human subject has a nucleotide sequence identical to or different from one or more reference sequences; and (d) performing a restriction endonuclease digestion to determine whether nucleic acid from the human subject has a nucleotide sequence identical to or different from one or more reference sequences.

A related aspect of the invention provides methods of assaying or screening for risk of developing hereditary lymphedema as described above, wherein the method comprises: performing a polymerase chain reaction (PCR) to amplify nucleic acid comprising the coding sequence of the LEC polynucleotide, and determining nucleotide sequence of the amplified nucleic acid.

Further provided by the invention is a method of screening for a hereditary lymphedema genotype in a human subject, comprising: (a) providing a biological sample comprising nucleic acid from said subject, and (b) analyzing the nucleic acid for the presence of a mutation altering the encoded amino acid sequence of the at least one allele of at least one gene in the human subject relative to a human gene encoding an amino acid sequence selected from the group consisting of SEQ ID NOS: 6, 8, 10, 12, 14, 16, 18, 20, 22, 24, 26, 28, 30, 32, 34, 36, 38, 40, 42, 43, 44, 45, and 46, wherein the presence of a mutation altering the encoded amino acid sequence in the human subject in a manner that correlates with lymphedema in human subjects identifies a hereditary lymphedema genotype. In some embodiments of this method, the biological sample is a cell sample. In other embodiments of this method, the analyzing comprises sequencing a portion of the nucleic acid. In still further embodiments of this method, the human subject has a hereditary lymphedema genotype identified by the method of screening.

In is contemplated that the above methods further comprise assaying the nucleic acid for a second mutation that correlates with a hereditary lymphedema phenotype and alters the encoded amino acid sequence of a second gene allele of the human subject when compared to the amino acid sequence of the polypeptide encoded by a corresponding wild-type allele of said second gene, wherein the wild-type polypeptide encoded by said second gene is a polypeptide identified in any of Tables 3 and 4 of PCT/US03/06900.

Another aspect of the invention provides a method of inhibiting lymphangiogenesis comprising administering to a subject an inhibitor of a LEC transmembrane polypeptide, wherein the LEC transmembrane polypeptide comprises an amino acid sequence selected from the group consisting of SEQ ID NOs: 6, 8, 10, 12, 14, 16, 20, 22, 24, 42 and 43, and wherein the inhibitor is selected from the group consisting of (a) a soluble extracellular domain fragment of the LEC transmembrane polypeptide; (b) an antibody that binds to the extracellular domain of the LEC transmembrane polypeptide; (c) a polypeptide comprising an antigen binding domain of the antibody according to (b); and (d) an antisense nucleic acid complementary to the nucleic acid encoding the LEC transmembrane polypeptide or its complement. In some embodiments of the method, the inhibitor is a polypeptide comprising an extracellular domain fragment of an LEC polypeptide. It is further contemplated that the sequence of said extracellular domain is selected from the group consisting of amino acids 1-1005 of SEQ ID NO: 43. In some embodiments of the method, the subject is a human containing a tumor.

In a related aspect, the invention provides a method for modulating lymphangiogenesis in a mammalian subject comprising: administering to a mammalian subject in need of modulation of lymphangiogenesis an antisense molecule to a LEC polynucleotide, in an amount effective to inhibit transcription or translation of the polypeptide encoded by the LEC polynucleotide, wherein the LEC polynucleotide comprises a nucleotide sequence selected from the group consisting of SEQ ID NOS: 5, 7, 9, 11, 13, 15, 17, 19, 21, 23, 25, 27, 29, 31, 33, 35, 37, 39 and 41. Antisense molecules of suitable sequence and length are known in the art, and include sequences perfectly complementary to a coding region of a coding strand of a nucleic acid, as well as lengths capable of relatively specific and stable hybridization to a target in vivo (e.g., lengths of each incremental integer in the range of 8-50, with a preferred length of at least 17 nucleotides).

The methods of the invention also include methods for the treatment of disorders related to lymphatic endothelial cells as recited above which may involve the administration of such compounds to individuals exhibiting symptoms or tendencies related to such disorders.

In another aspect, the invention provides a method of treating hereditary lymphedema, comprising: (a) identifying a human subject with hereditary lymphedema and with a mutation that alters the encoded amino acid sequence of at least one polypeptide of the human subject, relative to the amino acid sequence of a polypeptide comprising an amino acid sequence selected from the group consisting of SEQ ID NOS: 6, 8, 10, 12, 14, 16, 18, 20, 22, 24, 26, 28, 30, 32, 34, 36, 38, 40, 42, 43, 44, 45, and 46; and (b) administering to the subject a lymphatic growth factor selected from the group consisting of a VEGF-C polynucleotide, a VEGF-C polypeptide, a VEGF-D polynucleotide, and a VEGF-D polypeptide.

The invention also provides a method of treating hereditary lymphedema comprising: identifying a human subject with lymphedema and with a mutation in at least one allele of a gene encoding a LEC protein identified in any of Tables 1 and 2, wherein the mutation correlates with lymphedema in human subjects, and with the proviso that the LEC-specific protein is not VEGFR-3; and administering to the subject a composition comprising a lymphatic growth agent selected from the group consisting of VEGF-C polypeptides, VEGF-D polypeptides, VEGF-C polynucleotides, and VEGF-D polynucleotides. The invention also comprehends use of a lymphatic growth agent selected from the group consisting of VEGF-C polypeptides, VEGF-D polypeptides, VEGF-C polynucleotides, and VEGF-D polynucleotides in the manufacture of a medicament for the treatment of hereditary lymphedema resulting from a mutation in a LEC gene identified in any of Tables 1 and 2. It is contemplated that the method further comprises a mutation in at least one allele of a second gene encoding a LEC protein identified in any of Tables 3 and 4 of PCT/US03/06900, wherein the mutation in said second gene correlates with lymphedema in human subjects.

In addition, the invention encompasses methods for treating such diseases or disorders by administering compounds and other substances that modulate the overall activity of the target gene products. Compounds and other substances can effect such modulation either at the level of target gene expression or target protein activity. These treatment methods include the administration of a polypeptide or a polynucleotide according to the invention to an endothelial cell, e.g., a LEC and/or a BEC, or to an organism such as a human patient. An exemplary method according to this aspect of the invention is the administration of a therapeutic selected from the group consisting of an antisense polynucleotide capable of modulating the expression of at least one polynucleotide according to the invention, a polypeptide according to the invention, a polynucleotide according to the invention, an antibody or antibody fragment specifically recognizing a polypeptide according to the invention, a VEGF-C polynucleotide, a VEGF-C polypeptide, a VEGF-D polynucleotide, a VEGF-D polypeptide and a soluble VEGFR-3 polypeptide.

In another aspect, the invention provides a method of screening for an endothelial cell disorder or predisposition to the disorder, comprising obtaining a biological sample containing endothelial cell mRNA from a human subject; and measuring expression of a BEC or LEC gene from the amount of mRNA in the sample transcribed from the gene, wherein the BEC or LEC gene encodes a polypeptide identified in any of Tables 1 and 2. In a related aspect, the method further comprises a second BEC or LEC gene that encodes a polypeptide set forth in any of Tables 3 and 4 of PCT/US03/06900.

The invention also relates to a method of inhibiting the growth of a lymphatic endothelial cell, the method comprising contacting the cell with a composition comprising at least one antibody conjugated to an agent capable of inhibiting the growth, wherein the agent is selected from the group consisting of a cytotoxic agent and a cytostatic agent, and wherein the antibody specifically binds to a polypeptide encoded by a polynucleotide comprising a sequence selected from the group consisting of SEQ ID NOS: 5, 7, 9, 11, 13, 15, 19, 21 and 23. In specific embodiments of this method, the polypeptide comprises an amino acid sequence selected from the group consisting of SEQ ID NOS: 6, 8, 10, 12, 14, 16, 20, 22, 24, 42 and 43.

The invention further relates to methods of detecting a lymphatic endothelial cell, the method comprising contacting the cell with a composition comprising at least one antibody conjugated to a detectable agent, such as a fluorescent molecule or a radiolabeled molecule. In specific embodiments, the antibody specifically binds to a polypeptide encoded by a polynucleotide comprising a sequence selected from the group consisting of SEQ ID NOS: 5, 7, 9, 11, 13, 15, 19, 21 and 23. In further specific embodiments of this method, the polypeptide comprises an amino acid sequence selected from the group consisting of SEQ ID NOS: 6, 8, 10, 12, 14, 16, 20, 22, 24, 42 and 43.

The invention still further relates to methods of isolating a lymphatic endothelial cell, comprising contacting the cell with a solid matrix comprising at least one antibody capable of binding to a transmembrane protein in the cell membrane of the cell, and isolating cells specifically bound to the antibody matrix. In specific embodiments, the antibody specifically binds to a polypeptide encoded by a polynucleotide comprising a sequence selected from the group consisting of SEQ ID NOS: 5, 7, 9, 11, 13, 15, 19, 21 and 23. In further specific embodiments of this method, the polypeptide comprises an amino acid sequence selected from the group consisting of SEQ ID NOS: 6, 8, 10, 12, 14, 16, 20, 22, 24, 42 and 43.

The invention also relates to the administration of an agonist or antagonist to a lymphatic endothelial cell surface receptor, comprising selecting an antibody, a peptide or a small molecular weight compound that is capable of specifically binding to a lymphatic endothelial cell-specific protein, wherein the antibody, peptide or small molecular weight compound is an agonist or antagonist for a growth factor receptor, a cytokine receptor, a chemokine receptor, or a hemopoietic receptor, and contacting the antibody, peptide or small molecular weight compound with the lymphatic endothelial cell in need of growth stimulation or inhibition. In specific embodiments, such lymphatic endothelial cells are associated with lymphedema, lymphangioma, lymphangiomyeloma, lymphangiomatosis, lymphangiectasis, lymphosarcoma, and lymphangiosclerosis.

The invention also relates to the administration of a cytotoxic or cytostatic drug to a cancerous lymphatic endothelial cell, comprising selecting an antibody, a peptide or a small molecular weight compound that is capable of specifically binding to a lymphatic endothelial cell-specific protein, wherein the antibody, peptide or small molecular weight compound is complexed to the cytotoxic or cytostatic drug. In specific embodiments, administration of such complexes is useful in the treatment of malignant tumor diseases prone to metastatic spread through the lymphatic system.

The invention also provides a method of monitoring the efficacy or toxicity of a drug on endothelial cells, comprising the steps of measuring expression of at least one BEC or LEC gene in endothelial cells of a mammalian subject before and after administering a drug to the subject, wherein the at least one LEC or BEC gene encodes a polypeptide found in any of Tables 1 and 2 herein, and wherein changes in expression of the LEC or BEC gene correlates with efficacy or toxicity of the drug on endothelial cells. It is contemplated that the method further comprises a second BEC or LEC gene that encodes a polypeptide set forth in any of Tables 3 and 4 of PCT/US03/06900.

The invention further relates to a method of detecting a lymphatic endothelial cell, comprising contacting said cell with an antibody as described herein, wherein the antibody is detectably labeled.

The invention still further relates to a method of inhibiting at least one biological activity of a lymphatic endothelial cell, comprising contacting the cell with an agent capable of binding to at least one polypeptide encoded by any one of SEQ ID NOS: 6, 8, 10, 12, 14, 16, 20, 22, 24, 42 and 43, wherein the activity of the polypeptide is reduced relative to the activity of a polypeptide that is not contacted with the agent.

The invention also relates to a method of inhibiting the growth of a lymphatic endothelial cell, the method comprising contacting the cell with an antisense oligonucleotide capable of specifically binding to at least one polynucleotide selected from the group consisting of SEQ ID NOS: 5, 7, 9, 11, 13, 15, 17, 19, 21, 23, 25, 27, 29, 31, 33, 35, 37, 39 and 41. In a specific embodiment, the antisense oligonucleotide consists essentially of about 12 to about 25 contiguous nucleotides of any one of SEQ ID NOS: 5, 7, 9, 11, 13, 15, 17, 19, 21, 23, 25, 27, 29, 31, 33, 35, 37, 39 and 41.

It is further contemplated that the methods of the invention, such as identifying a LEC nucleic acid; modulating the growth of lymphatic endothelial cells in a human subject; identifying compounds that modulate growth of endothelial cells; identifying a human subject with a disorder characterized by hyperproliferation of LECs; assaying for risk of developing hereditary lymphedema; screening for an increased risk of developing hereditary lymphedema; screening for a hereditary lymphedema genotype in a human subject; and inhibiting lymphangiogenesis, may be carried out using compositions comprising polypeptide fragments as described above. In some embodiments, the polypeptide is at least 95% identical to a polypeptide set forth herein, comprising a particular biological activity associated with a protein domain as set forth herein. The polypeptide may have transcription factor activity or cytoskeletal regulation activity. Additional exemplary biological activities include the ubitquitin ligase activity of a Hect domain, guanine-nucleotide-dissociation-stimulating activity of at least one RCC1-like domain, cation transport activity of a cation transport domain, GTP-binding activity of at least one GTP-binding domain, SPRY domain activity of at least one SPRY domain, and Kelch domain activity of at least one Kelch domain. Variations on the methods above may also be carried out using the above-described polypeptide fragment compositions.

Additional features and variations of the invention will be apparent to those skilled in the art from the entirety of this application, and all such features are intended as aspects of the invention.

### DETAILED DESCRIPTION OF THE INVENTION

A major role of the lymphatic vasculature is to remove an excess of the protein-rich interstitial fluid that continuously escapes from the blood capillaries, and to return it to the blood circulation (Witte, M. H., et al., Microsc. Res. Tech. 55:122-145. 2001; Karpanen, T., et al., J. Exp. Med. 194:F37-F42. 2001; Karkkainen, M. J., et al., Trends Mol. Med. 7:18-22, 2001). In addition, the lymphatic system provides constant immune surveillance by filtering lymph and its antigens through the chain of lymph nodes, and also serves as one of the major routes for absorption of lipids from the gut. It has been known for a long time that in many types of cancer the lymphatic vessels provide a major pathway for tumor metastasis, and regional lymph node dissemination correlates with the progression of the disease. Hereditary lymphedema, post-surgical secondary lymphedema and lymphatic obstruction in filariasis, are all characterized by disabling and disfiguring swelling of the affected areas, linked to the insufficiency or obstruction of the lymphatics. Witte, M. J., et al., Microsc. Res. Tech 55:122-145 (2001).

In spite of the importance of lymphatic vessels in medicine, the cell biology of this part of the vascular system has received little attention until recently. Studies during the past four years have uncovered the existence of the lymphatic specific vascular endothelial growth factors VEGF-C and VEGF-D, which serve as ligands for the receptor tyrosine kinase VEGFR-3, and demonstrated their importance for the normal development of the lymphatic vessels (See, Jeltsch et al., Science 276:1423-1425 (1997); Veikkola, T., et al., EMBO J. 20:1223-1231 (2001); Mäkinen, T., et al., Nat. Med. 7:199-205 (2001)). These molecules also appear to be involved in the development of lymphedema and lymphatic metastasis (Karpanen et al., J. Exp. Med. 194:F37-F42 (2001); Karkkainen et al., Trends Mol. Med. 7:18-22, 2001).

The growth factor Vascular Endothelial Growth Factor C (VEGF-C), as well as native human, non-human mammalian, and avian polynucleotide sequences encoding VEGF-C, and VEGF-C variants and analogs, have been described in detail in International Patent Application Number PCT/US98/01973, filed Feb. 2, 1998 and published on Aug. 6, 1998 as International Publication Number WO 98/33917; in Joukov et al., J. Biol. Chem., 273(12): 6599-6602 (1998); and in Joukov et al., EMBO J., 16(13): 3898-3911 (1997), all of which are incorporated herein by reference in their entirety. As explained therein in detail, human VEGF-C (SEQ ID NOS: 1 and 2) is initially produced in human cells as a prepro-VEGF-C polypeptide of 419 amino acids. A cDNA encoding human VEGF-C (SEQ ID NO: 1) has been deposited with the American Type Culture Collection (ATCC), 10801 University Blvd., Manassas, Va. 20110-2209 (USA), pursuant to the provisions of the Budapest Treaty (Deposit date of 24 Jul. 1995 and ATCC Accession Number 97231). VEGF-C sequences from other species also have been reported. See Genbank Accession Nos. MMU73620 (Mus musculus); and CCY15837 (Coturnix coturnix) for example, incorporated herein by reference.

The prepro-VEGF-C polypeptide is processed in multiple stages to produce a mature and most active VEGF-C polypeptide of about 21-23 kD, as assessed by SDS-PAGE under reducing conditions (SEQ ID NO: 2). Such processing includes cleavage of a signal peptide (residues 1-31); cleavage of a carboxyl-terminal peptide (corresponding approximately to amino acids 228-419 and having a pattern of spaced Cysteine residues reminiscent of a Balbiani ring 3 protein (BR3P) sequence [Dignam et al., Gene, 88:133-40 (1990); Paulsson et al., J. Mol. Biol., 211:331-49 (1990)]) to produce a partially-processed form of about 29 kD; and cleavage (apparently extracellularly) of an amino-terminal peptide (corresponding approximately to amino acids 32-103) to produced a fully-processed mature form of about 21-23 kD. Experimental evidence demonstrates that partially-processed forms of VEGF-C (e.g., the 29 kD form) are able to bind VEGFR-3 (Flt4 receptor), whereas high affinity binding to VEGFR-2 occurs only with the fully processed forms of VEGF-C. It appears that VEGF-C polypeptides naturally associate as non-disulfide linked dimers.

It has been demonstrated that amino acids 103-227 of VEGF-C are not all critical for maintaining VEGF-C functions. A polypeptide consisting of amino acids 113-213 (and lacking residues 103-112 and 214-227) retains the ability to bind and stimulate VEGF-C receptors, and it is expected that a polypeptide spanning from about residue 131 to about residue 211 will retain VEGF-C biological activity. The Cysteine residue at position 156 has been shown to be important for VEGFR-2 binding ability. However, VEGF-C ΔC₁₅₆ polypeptides (i.e., analogs that lack this Cysteine due to deletion or substitution) remain potent activators of VEGFR-3. The Cysteine at position 165 of VEGF-C polypeptide is essential for binding either receptor, whereas analogs lacking the Cysteine at positions 83 or 137 compete with native VEGF-C for binding with both receptors and stimulate both receptors.

VEGF-D is structurally and functionally most closely related to VEGF-C (see U.S. Pat. No. 6,235,713 and International Patent Publ. No. WO 98/07832, incorporated herein by reference). See SEQ ID NO: 3 for the polynucleotide sequence of VEGF-D; the encoded amino acid sequence is set forth in SEQ ID NO: 4. Like VEGF-C, VEGF-D is initially expressed as a prepro-peptide that undergoes N-terminal and C-terminal proteolytic processing, and forms non-covalently linked dimers. VEGF-D stimulates mitogenic responses in endothelial cells in vitro. During embryogenesis, VEGF-D is expressed in a complex temporal and spatial pattern, and its expression persists in the heart, lung, and skeletal muscles in adults. Isolation of a biologically active fragment of VEGF-D designated VEGF-DΔNΔC, is described in International Patent Publication No. WO 98/07832, incorporated herein by reference. VEGF-DΔNΔC consists of amino acid residues 93 to 201 of VEGF-D (SEQ ID NO: 4) optionally linked to the affinity tag peptide FLAG®, or other sequences.

The prepro-VEGF-D polypeptide has a putative signal peptide of 21 amino acids and is apparently proteolytically processed in a manner analogous to the processing of prepro-VEGF-C. A “recombinantly matured” VEGF-D lacking residues 1-92 and 202-354 retains the ability to activate receptors VEGFR-2 and VEGFR-3, and appears to associate as non-covalently linked dimers. Thus, preferred VEGF-D polynucleotides include those polynucleotides that comprise a nucleotide sequence encoding amino acids 93-201. The guidance provided above for introducing function-preserving modifications into VEGF-C polypeptides is also suitable for introducing function-preserving modifications into VEGF-D polypeptides. As another aspect of the invention, practice of the invention methods is contemplated wherein VEGF-D polypeptides are employed in lieu of VEGF-C polypeptides.

When compared with the blood vascular endothelium, the lymphatic endothelium exhibits specific morphological and molecular characteristics. For example, the lymphatic capillaries are larger than blood capillaries, they have an irregular or collapsed lumen with no red blood cells, a discontinuous basal lamina, overlapping intercellular junctional complexes and anchoring filaments that connect the lymphatic endothelial cells to the extracellular matrix (Witte, M. H., et al., Microsc. Res. Tech. 55:122-145 (2001)). Unlike the blood capillaries, the lymphatic capillaries lack pericyte coverage. At the molecular level several lymphatic specific markers have been identified, including VEGFR-3, the Prox-1 transcription factor, the hyaluronan receptor LYVE-1, the membrane mucoprotein podoplanin, the beta-chemokine receptor D6, the cytoskeletal proteins desmoplakin I and II and the macrophage mannose receptor I (Wigle, J. T. & Oliver, G., Cell 98:769-778 (1999); Banerji, S., et al., J. Cell Biol. 144:789-801 (1999): Breiteneder-Geleff, S., et al., Am. J. Pathol. 154:385-394 (1999): Nibbs, R. J., et al., Am. J. Pathol. 158:867-877 (2001); Ebata, N., et al., Microvasc. Res. 61:40-48. (2001); Irjala, H., et al., J. Exp. Med 194:1033-1041 (2001)). The present invention relates to the genetic identity of lymphatic capillary endothelial cells versus blood vascular endothelial cells using a gene profiling approach.

“Stringent hybridization conditions” or “stringent conditions” refer to conditions under which a nucleic acid such as an oligonucleotide will specifically hybridize to its target sequence. Stringent conditions are sequence-dependent and will be different in different circumstances. Longer nucleic acids hybridize specifically at higher temperatures than shorter sequences. Generally, stringent conditions are selected to be about 5° C. lower than the thermal melting point (Tₘ) for the specific sequence at a defined ionic strength and pH. The Tₘ is the temperature (under defined ionic strength, pH and nucleic acid concentration conditions) at which 50% of the nucleic acids complementary to the target sequence hybridize to the target sequence at equilibrium. The term “complementary” refers to standard Watson-Crick base pairing between nucleotides of two nucleic acid molecules. Typically, stringent conditions will be those in which the salt concentration is less than about 1.0 M sodium ion, typically about 0.01 to 1.0 M sodium ion (or other salts) at pH 7.0 to 8.3 and at a temperature that is at least about 30° C. for short probes, primers or oligonucleotides (e.g., 10 to 50 nucleotides) and at least about 60° C. for longer probes, primers or oligonucleotides. Stringent conditions also can be achieved with the addition of destabilizing agents, such as formamide, as is known in the art Exemplary stringent hybridization conditions are hybridization at 42° C. for 20 hours in a solution containing 50% formamide, 5×SSPE, 5× Denhardt's solution, 0.1% SDS and 0.1 mg/ml denatured salmon sperm DNA, with a wash in 1×SSC, 0.1% SDS for 30 minutes at 65° C.

Distinct gene expression profiles for blood vascular and lymphatic endothelial cells have been discovered (See PCT/US03/06900, incorporated herein by reference). These results provide new insights into the phenotypic diversity of endothelial cells and reveal new potential lymphatic endothelial cell molecules, some of which could provide important targets for the therapy of diseases characterized by abnormal angiogenesis or lymphangiogenesis.

Differences in the expression of genes encoding proteins involved in inflammatory processes were found, as well as in those mediating cell-cell and cell-matrix interactions. Furthermore, several previously unknown genes were identified in the context of endothelial cell biology, which were differentially expressed in the two cell lineages. Several of the genes were originally cloned from neural tissues, including genes involved in the uptake of synaptic macromolecules and in synapse formation and remodeling (neuronal pentraxins I and II (Kirkpatrick, L. L., et al., J. Biol. Chem. 275:17786-17792. 2000), in the trafficking of synaptic vesicles (NAP-22 (Yamamoto, Y., et al., Neurosci. Lett. 224:127-130. 1997), piccolo (Fenster, S. D., et al., Neuron 25:203-214 (2000)) and in the axon growth and guidance (Nr-CAM (Grumet, M., Cell Tissue Res. 290:423-428 (1997), reelin (Rice, D. S. & Curran, T., Annu. Rev. Neurosci. 24:1005-1039 (2001)).

In addition, the LECs especially expressed a number of uncharacterized genes, which were originally cloned and highly expressed in nervous tissues (KIAA genes (Kikuno, R., et al., Nucleic Acids Res. 30:166-168. 2002)). The gene expression profiling data disclosed herein therefore support the view that the same molecular mechanisms that are involved in governing neural cell positioning, in guiding axonal growth cones to their specific targets and in synaptogenesis may also be commonly used in the development of the vascular system and in the establishment of BEC and LEC identity. Some other signaling molecules first described in the developing nervous system have already been implicated in the development of the vasculature and vice versa (Shima and Mailhos, Curr. Opin Genet. Dev. 10.536-542 (2000); Oosthuyse, et al., Nat. Genet. 28:131-138 (2001); Sondell, et al., Eur. J. Neurosci. 12:4243-4254 (2000)).

PCT/US03/06900 identified expression of several genes in the LECs, previously shown to be expressed in smooth muscle cells (SMCs) and pericytes, such as matrix Gla, a mineral binding extracellular matrix protein involved in the inhibition of vascular and tissue calcification (Luo, G., et al., Nature 386:78-81 (1997)), monoamine oxidase A, the main degradative enzyme of monoamine hormones and neurotransmitters (Rodriguez, M. J., et al., Cell Tissue Res. 304:215-220 (2001)), integrin α9 (Palmer, E. L., et al., J. Cell Biol. 123:1289-1297 (1993)) and apolipoprotein D (Hu, C. Y., et al., J. Neurocytol. 30:209-218 (2001)). Some similarity of gene expression patterns between LECs and SMCs could be related to the lack of SMC around lymphatic capillaries. Instead, LECs may carry out some SMC functions by themselves. For example, lymph flow is maintained due to the intrinsic contractility of the LECs (Witte, M. H., et al., Microsc. Res. Tech. 55:122-145 (2001)), reminiscent of the ability of vascular SMCs to contract

Molecular discrimination of the lymphatic and blood vessels is essential in studies of diseases involving the blood and/or lymphatic vessels and in the targeted treatment of such diseases. To date, several lymphatic endothelial specific markers have been identified, but some of them are expressed only in a subset of the lymphatic vessels, while others also occur in some blood vessel endothelia or in other cell types and their expression patterns may change in pathological conditions (for example, VEGFR-3 (Valtola, R., et al., Am. J. Pathol. 154:1381-1390.1999)). Identification of new vascular markers according to the invention should provide a more reliable analysis of the blood and lymphatic vessels in pathological situations and eventually better diagnosis and treatment. Furthermore, inhibition of the function of certain molecules involved in the regulation of angiogenesis and/or lymphangiogenesis is known to prevent tumor growth and metastasis, and stimulation of the growth of blood or lymphatic vessels has been shown to be beneficial in several pathological conditions. Thus the LEC specific molecular regulators identified according to the invention may provide new targets for the treatment of diseases characterized by abnormal angiogenesis and lymphangiogenesis.

Several of the new LEC genes encode transmembrane proteins which are expected to be specific molecular markers for lymphatic endothelial cells (Table 1). These genes and encoded proteins are useful for targeted treatment of diseases that involve lymphatic vessels. They may also be useful for preparing antibodies, as antibodies against LEC-specific proteins can be used to discriminate between blood and lymphatic vessels in pathological and physiological situations. Antibodies may also be useful for the isolation of lymphatic endothelial cells. These proteins may also play a role in the regulation of lymphangiogenesis, and can provide new candidate genes for diseases that involve lymphatic vessels, such as lymphedema

The lymphatic endothelial cell specific surface molecules can be used for molecular drug targeting with antibodies, peptides and small-molecular weight compounds, which can act as agonists or antagonists for growth factor receptor, cyto- and chemokine receptor, and hemopoietin receptor signaling, cell adhesion and cell interaction with extracellular matrix or with other cell surface molecules. Such molecules can also be used for targeting of cytotoxic or cytostatic drugs into the lymphatic endothelial cells and for the attachment of electron-dense, radio-opaque or radioactive markers for imaging of disease processes associated with the lymphatic vessels. Such diseases include lymphedema, lymphangioma, lymphangiomyoma, lymphangiomatosis, lymphangiectasis, lymphosarcoma and lymphangiosclerosis.

The lymphatic endothelial cell surface molecules may be used for targeting of gene therapy for example by antibody-coated liposomes (containing proteins or genes as cargo) or by viral transducing vectors such as adenoviruses, adeno-associated viruses or lentiviruses having modified capsid/envelope proteins. The manipulation of lymphatic endothelial cell specific molecules may be applicable to treatment of disease processes associated with tissue edema by increasing fluid transport across the lymphatic vessel wall for example by modulating endothelial cell-cell or cell-matrix interactions or via stimulating transendothelial transport. Targeting of the lymphatic endothelial cells for example with cytotoxic or cytostatic compounds is contemplated to be valuable in malignant tumor diseases prone to metastatic spread via the lymphatic system.

The lymphatic endothelial cell molecules may allow the improved in vitro growth of lymphatic endothelial cells as well as in vitro tissue engineering of lymphatic vessels for use in diseases where the lymphatics have been damaged, such as after surgery and in various forms of lymphedema. Ligands of the cell surface proteins may further be applied to coat various polymeric matrices for the adhesion of cells in, e.g., bioimplants.

The lymphatic endothelial-cell-specific molecules such as surface molecules can provide important tools for the modulation of inflammatory, autoimmune and infectious processes involving leukocyte migration and immune recognition as well as the stimulation of secondary immune responses. Such processes include the migration of antigen presenting cells into the lymphatic system including lymph nodes as well as transendothelial cell trafficking of lymphocytes and other leukocyte subclasses and the homing, survival and function of the various classes of leukocytes.

These molecules may allow one to modulate the metabolism of fatty acids including fatty acid/chylomicron absorption from the gut and regulation of fat accumulation in adipose tissue in various organs such as in the subcutaneous tissue and in the arterial wall.

Lymphatic endothelial-cell-specific molecules may further allow one to modulate the metabolism of fatty acids including fatty acid/chylomicron absorption from the gut and regulation of fat accumulation in adipose tissue in various organs such as in the skin subcutaneous tissue and in the arterial wall.

The lymphatic-cell-specific transmembrane proteins are expected to function in cell adhesion (e.g., adhesion between lymphatic endothelial cell-lymphatic endothelial cell, lymphatic endothelial cell-smooth muscle cell, lymphatic endothelial cell-immune system cell such as lymphocyte or dendritic cell), cell-extracellular matrix contacts, or as receptors such as growth factor, cytokine, chemokine or microbial receptors or ion channels. The transmembrane (TM) proteins connect to intracellular molecules that can induce cell growth, cell migration, cell apoptosis, cell differentiation or cell adhesion or other cellular functions specific for endothelial cells such as expression of adhesion receptors for leukocytes, release of nitric oxide, antigoagulant proteins, uptake of fluid and proteins from surrounding tissues and fat from gut or adipose tissues. TM proteins with short intracellular domains can function as auxiliary receptors in complex with other TM proteins.

The transmembrane proteins and their intracellular binding partner molecules can be used as molecular markers for lymphatic endothelial cells in normal and disease conditions, and to discriminate between blood and lymphatic vessels in pathological and physiological situations.

Antibodies against lymphatic specific transmembrane proteins, as well as peptides and small molecular compounds binding to extracellular domains of lymphatic-specific TM proteins can be used for the attachment of electron-dense, radio-opaque or radioactive markers for imaging of disease processes associated with the lymphatic vessels. Such diseases include lymphedema, lymphangioma, lymphangiomyoma, lymphangiomatosis, lymphangiectasis, lymphosarcoma and lymphangiosclerosis. Similarly, the lymphatic vessels can be visualized e.g., during therapy of patients suffering from insufficient lymphatic growth, such as in lymphedema, or alternatively during treatment aiming to prevent lymphatic growth, e.g., in tumors, thereby facilitating the monitoring of the therapeutic method of the invention.

Antibodies against LEC-specific TM proteins are also expected to be useful for the isolation of lymphatic endothelial cells.

Antibodies against lymphatic-specific transmembrane proteins, or peptides or small-molecule compounds binding to the extracellular domain of lymphatic-specific TM proteins are expected to be useful in targeting drug delivery to lymphatic endothelial cells, e.g., by coupling an antibody, peptide or small-molecule compound to a cytotoxic or cytostatic compound. Such coupled compounds are useful as therapeutics in the treatment of malignant tumor diseases prone to metastatic spread via the lymphatic system, as well as in ameliorating a symptom associated with any such disease. The antibodies, peptides or small-molecule compounds can also be coupled to stimulatory lymphatic endothelial molecules such as growth factors, cytokines and chemokines to promote stimulation.

Additionally, antibodies against lymphatic-specific TM proteins or peptides, or small-molecule compounds binding to the extracellular domain of lymphatic-specific TM proteins, may be used for targeting of gene therapy, for example, by antibody-coated liposomes (containing proteins, genes or other molecules as cargo) or by viral transducing vectors such as adenoviruses, adeno-associated viruses, lentiviruses, or the like, having modified capsid/envelope proteins The manipulation of lymphatic endothelial-cell-specific molecules are expected to be applicable to the treatment of disease processes associated with tissue edema due to the relative absence, or relative dysfunction, of lymphatic vessels, which can result from an infection, surgery, radiotherapy or a genetic defect by increasing fluid transport across the lymphatic vessel wall, for example by modulating endothelial cell-cell or cell-matrix interactions or by stimulating transendothelial transport.

The lymphatic endothelial cell molecules are expected to improve the in vitro growth of lymphatic endothelial cells, as well as the in vitro tissue engineering of lymphatic vessels for use in treating disorders or diseases where the lymphatics have been damaged, such as after surgery, in various forms of lymphedema, and in other applications as described herein. Ligands of the cell-surface proteins may further be applied as a coating to various polymeric matrices for the adhesion of cells in, e.g., bioimplants.

Inflammatory, autoimmune and infectious processes involving leukocyte migration and immune recognition, such as migration of antigen-presenting cells into the lymphatic system, including lymph nodes, as well as transendothelial cell trafficking of lymphocytes and other leukocyte subclasses and the homing, survival and function of the various classes of leukocytes can be modulated by targeting endothelial-cell-specific TM proteins, which mediate these cell adhesion processes.

Upregulation of lymphatic-specific genes in, e.g., cancer are expected to be useful as diagnostic markers, and monitoring such upregulated expression with an antibody against a lymphatic endothelial-cell-specific protein, e.g., by immunostaining of tissue(s) or by using a probe hybridizable to a lymphatic endothelial-cell-specific mRNA, e.g., under stringent hybridization conditions as described herein, is contemplated.

Lymphatic endothelial-cell-specific transcription factors are expected to be useful for the differentiation of lymphatic endothelial cells from embryonic stem cells, endothelial precursor cells, or blood vascular endothelial cells.

The lymphatic endothelial transcription factors are expected to improve the in vitro growth of lymphatic endothelial cells, as well as to facilitate in vitro tissue engineering of lymphatic vessels for use in treating disorders or diseases where the lymphatics have been damaged, such as after surgery, in various forms of lymphedema, and in other applications disclosed herein.

Intracellular signaling proteins participating in signaling pathways regulating lymphatic endothelial cell proliferation, differentiation, apoptosis, migration or adhesion are expected to be useful targets for small-molecule compounds inhibiting these signaling events, and cellular functions dependent on such signaling. Signaling proteins are also expected to participate in VEGFR-3 signaling pathways, and will be useful in modulating cellular activities controlled, at least in part, by VEGFR-3 signaling, such as lymphangiogenesis.

The lymphatic endothelial cell molecules are expected to improve the in vitro growth of lymphatic endothelial cells as well as in vitro tissue engineering of lymphatic vessels for use in treating diseases or disorders where the lymphatics have been damaged, such as after surgery, in various forms of lymphedema, and in other applications as described herein.

Lymphatic-specific transcription factors are also expected to be useful in modulating gene expression in endothelial cells to induce the expression of other lymphatic-specific genes in, for example, blood vascular endothelial cells or endothelial precursor cells. Lymphatic-specific gene transcripts are expected to provide useful targets for RNA interference (RNAi)-induced inhibition of expression. RNAi technology is expected to be useful in the methods according to the invention, such as therapeutic methods effective in treating hyper- and hypo-proliferative endothelial-cell-associated diseases and disorders, as well as methods of ameliorating a symptom of any such disease or disorder. RNAi methodologies are known in the art and known RNAi technologies are contemplated as useful in various aspects of the invention. See Fire et al., Nature 391:806-811. (1998) and Sharp, P., Genes and Dev. 13:139-141. (1999), each of which is incorporated herein by reference. It is preferred that RNAi compounds be double-stranded RNA molecules corresponding to part or all of a coding region of a desired target for expression.

As noted, several of the new LEC genes encode transcription factors, may control cellular fate and have an important role in the differentiation of lymphatic endothelial cells. Transcription factors disclosed herein (Table 2) may control transcription of genes involved for example in the proliferation of lymphatic endothelial cells, and may be important molecular regulators of lymphatic growth. Lymphatic endothelial cell specific transcription factors can be used for the differentiation of lymphatic endothelial cells from embryonic stem cells, endothelial precursor cells or from blood vascular endothelial cells.

The lymphatic endothelial transcription factors may allow the improved in vitro growth of lymphatic endothelial cells as well as in vitro tissue engineering of lymphatic vessels for use in diseases where the lymphatics have been damaged, such as after surgery and in various forms of lymphedema.

Polynucleotides of the Invention

In general, the isolated polynucleotides of the invention include the LEC and BEC polynucleotides exhibiting differential expression and identified in Tables 1 and 2, including sequences identifiers of these polynucleotides and their known database accession numbers, where applicable. The polynucleotide sequences may include a coding region and may include non-coding flanking sequences, which are readily identifiable by one of skill in the art. The invention contemplates polynucleotides comprising part, or all, of a coding region, with or without flanking regions, e.g., poly A sequences, 5′ non-coding sequences, and the like. The polynucleotides of the present invention also include, but are not limited to, a polynucleotide that hybridizes to the complement of the nucleotide sequence of any one of SEQ ID NOS: 5, 7, 9, 11, 13, 15, 17, 19, 21, 23, 25, 27, 29, 31, 33, 35, 37, 39 and 41 under highly stringent hybridization conditions; a polynucleotide that hybridizes to the complement of the nucleotide sequence of any one of SEQ ID NOS: 5, 7, 9, 11, 13, 15, 17, 19, 21, 23, 25, 27, 29, 31, 33, 35, 37, 39 and 41 under moderately stringent hybridization conditions; a polynucleotide which is an allelic variant of any polynucleotide recited above; a polynucleotide which encodes a species homologue of any of the proteins recited above; or a polynucleotide that encodes a polypeptide comprising a specific domain or truncation of the polypeptide of any one of SEQ ID NOS: 6, 8, 10, 12, 14, 16, 18, 20, 22, 24, 26, 28, 30, 32, 34, 36, 38, 40, 42, 43, 44, 45, and 46. Such polynucleotides hybridize under the above conditions to the complement of any one of SEQ ID NOS: 5, 7, 9, 11, 13, 15, 17, 19, 21, 23, 25, 27, 29, 31, 33, 35, 37, 39 and 41 or to a fragment of any one of SEQ ID NOS: 5, 7, 9, 11, 13, 15, 17, 19, 21, 23, 25, 27, 29, 31, 33, 35, 37, 39 and 41 wherein the fragment is greater than at least about 10 bp, and, in alternate embodiments, is about 20 to about 50 bp, or is greater than about 100 bp, 200 bp, 300 bp, 400 bp, 500 bp, 600 bp, 700 bp, or 800 bp, where appropriate. References to “bp” refer to the length of double-, or single-, stranded nucleic acids in nucleotides or base pairs, as would be apparent from the context.

The polynucleotides of the invention also provide polynucleotides that are variants of the polynucleotides recited above. Typically, such a variant sequence varies from one of those listed herein by no more than about 20%, i.e., the number of individual nucleotide substitutions, additions, and/or deletions in a similar sequence, as compared to the corresponding reference sequence, divided by the total number of nucleotides in the variant sequence is about 0.2 or less. Such a sequence is said to have 80% sequence identity to the listed sequence. Such a variant sequence can be routinely identified by applying the foregoing algorithm.

In one embodiment, a variant polynucleotide sequence of the invention varies from a listed sequence by no more than 10%, i.e., the number of individual nucleotide substitutions, additions, and/or deletions in a variant sequence, as compared to the corresponding reference sequence, divided by the total number of nucleotides in the variant sequence is about 0.1 or less. Such a sequence is said to have 90% sequence identity to the listed sequence. Such a variant sequence can be routinely identified by applying the foregoing algorithm.

In an alternate embodiment a variant sequence of the invention varies from a listed sequence by no more than by no more than 5%, i.e., the number of individual nucleotide substitutions, additions, and/or deletions in a variant sequence, as compared to the corresponding reference sequence, divided by the total number of nucleotides in the variant sequence is about 0.05 or less. Such a sequence is said to have 95% sequence identity to the listed sequence. Such a variant sequence can be routinely identified by applying the foregoing algorithm.

In yet another alternate embodiment, a variant sequence of the invention varies from a listed sequence by no more than 2%, i.e., the number of individual nucleotide substitutions, additions, and/or deletions in a variant sequence, as compared to the corresponding reference sequence, divided by the total number of nucleotides in the variant sequence is about 0.02 or less. Such a sequence is said to have 98% sequence identity to the listed sequence. Such a variant sequence can be routinely identified.

A polynucleotide according to the invention can be joined to any of a variety of other nucleotide sequences by well-established recombinant DNA techniques (see Sambrook, J. et al. (2d Ed.; 1989) Molecular Cloning: A Laboratory Manual, Cold Spring Harbor Laboratory, Cold Spring Harbor, N.Y.). Useful nucleotide sequences for joining to polypeptides include an assortment of vectors, e.g., plasmids, cosmids, lambda phage derivatives, phagemids, and the like, that are well known in the art. Accordingly, the invention also provides a vector including a polynucleotide of the invention and a host cell containing the polynucleotide. In general, the vector contains an origin of replication functional in at least one organism, convenient restriction endonuclease sites, and a selectable marker for the host cell. Vectors according to the invention include expression vectors, replication vectors, probe generation vectors, sequencing vectors, and retroviral vectors. A host cell according to the invention can be a prokaryotic or eukaryotic cell and can be a unicellular organism or part of a multicellular organism. Large numbers of suitable vectors and promoters are known to those of skill in the art and are commercially available for generating the recombinant constructs of the present invention.

The sequences falling within the scope of the present invention are not limited to the specific sequences herein described, but also include allelic variations thereof. Allelic variations can be routinely determined by comparing the polynucleotide sequence provided in any one of SEQ ID NOS: 5, 7, 9, 11, 13, 15, 17, 19, 21, 23, 25, 27, 29, 31, 33, 35, 37, 39 and 41, a representative intermediate fragment thereof, or a nucleotide sequence at least 99.9% identical to any one of SEQ ID NOS: 5, 7, 9, 11, 13, 15, 17, 19, 21, 23, 25, 27, 29, 31, 33, 35, 37, 39 and 41 with a sequence from another isolate of the same species. Furthermore, to accommodate codon variability, the invention includes nucleic acid molecules coding for the same amino acid sequences as do the specific open reading frames (ORFs) disclosed herein. In other words, in the coding region of an ORF, substitution of one codon for another which encodes the same amino acid is expressly contemplated.

Unless provided for otherwise here, all terms are defined as is known in the art, for example as employed in U.S. Pat. No. 6,350,447, incorporated herein by reference.

Also contemplated are antisense polynucleotides based on the sequence of any of the LEC or BEC polynucleotides set forth found in any of Tables 1 and 2 herein or Tables 3 and 4 of PCT/US03/06900. Such antisense polynucleotides are substantially complementary (e.g., at least 90% complementarity), and preferably perfectly complementary, to any one of the polynucleotides of the invention, or fragments thereof, set out in any of Tables 1 and 2 herein or Tables 3 and 4 of PCT/US03/06900 (incorporated herein by reference) that are differentially expressed in LECs and BECs. These polynucleotide sequences include any of SEQ ID NOS: 5, 7, 9, 11, 13, 15, 17, 19, 21, 23, 25, 27, 29, 31, 33, 35, 37, 39 and 41, or a fragment thereof comprising at least 10 contiguous nucleotides. Antisense nucleic acid comprises a nucleotide sequence that is complementary to a “sense” nucleic acid encoding a protein (e.g., complementary to the coding strand of a double-stranded cDNA molecule or complementary to an mRNA sequence). Methods for designing and optimizing antisense nucleotides are described in Lima et al., (J Biol Chem, ;272:626-38. 1997) and Kurreck et al., (Nucleic Acids Res., ;30:1911-8. 2002). In one aspect, antisense nucleic acid molecules are provided that comprise a sequence complementary to at least about 10, 25, 50, 100, 250 or 500 nucleotides or an entire coding strand. An antisense nucleic acid of the invention can be constructed using chemical synthesis or enzymatic ligation reactions using procedures known in the art.

In one embodiment, an antisense nucleic acid molecule is antisense to a “coding region” of the coding strand of a nucleotide sequence encoding a polypeptide of the invention. The term “coding region” refers to the region of the nucleotide sequence comprising codons which are translated into amino acid residues. In another embodiment, the antisense nucleic acid molecule is antisense to a “conceding region” of the coding strand of a nucleotide sequence encoding the polynucleotide. The term “conceding region” refers to 5′ and 3′ sequences which flank the coding region that are not translated into amino acids (i.e., also referred to as 5′ and 3′ untranslated regions).

Antisense nucleic acids of the invention can be designed according to the rules of Watson and Crick or Hoogsteen base pairing. The antisense nucleic acid molecule can be complementary to the entire coding region of the mRNA of the polynucleotide of the invention, but more preferably is an oligonucleotide that is antisense to only a portion of the coding or noncoding region of the mRNA. An antisense oligonucleotide can be, for example, about 5, 10, 15, 20, 25, 30, 35, 40, 45, or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

The antisense nucleic acid molecules of the invention are typically administered to a subject or generated in situ such that they hybridize or bind to cellular mRNA and/or genomic DNA encoding the complementary polynucleotide, thereby inhibiting expression of the protein (e.g., by inhibiting transcription and/or translation). The hybridization can reflect conventional nucleotide complementarity to form a stable duplex, or, for example, in the case of an antisense nucleic acid molecule that binds to DNA duplexes, through specific interactions in the major groove of the double helix.

An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). Additional routes of antisense therapy may be used in the invention, e.g., topical administration, transdermal administration [reviewed by Brand in Curr. Opin. Mol. Ther. 3:244-8, 2001] antisense administration using nanoparticulate systems [Lambert et al., Adv. Drug. Deliv. Rev. 47:99-112, 2001], or administration of antisense nucleotides conjugated with peptide [Juliano et al., Curr. Opin. Mol. Ther. 2:297-303, 2000].

The invention further contemplates use of the polynucleotides of the invention for gene therapy or in recombinant expression vectors which produce polynucleotides or polypeptides of the invention that can regulate an activity of LEC genes, and are useful in therapy of LEC disorders such as lymphedema. Delivery of a functional gene encoding a polypeptide of the invention to appropriate cells is effected ex vivo, in situ, or in vivo by use of vectors, including viral vectors (e.g., adenovirus, adeno-associated virus, or a retrovirus), or ex vivo by use of physical DNA transfer methods (e.g., liposomes or chemical treatments). See, for example, Anderson, Nature, supplement to vol. 392, no. 6679, pp. 25-20 (1998). For additional reviews of gene therapy technology see Friedmann, (Science, 244: 1275-1281. 1989); Verma, (Scientific American: 263:68-72, 81-84. 1990); and Miller, (Nature, 357: 455-460. 1992). Introduction of any one of the nucleotides of the present invention or a gene encoding a polypeptide of the invention can also be accomplished with extrachromosomal substrates (transient expression) or artificial chromosomes (stable expression). Cells may also be cultured ex vivo in the presence of proteins of the present invention in order to proliferate or to produce a desired effect on, or activity in, such cells. In another embodiment, cells comprising vectors expressing the polynucleotides or polypeptides of the invention may be cultured ex vivo and administered to an individual in need of treatment for an LEC disease or disorder.

Given the foregoing disclosure of the nucleic acid constructs, it is possible to produce the gene product of any of the genes comprising the sequence of any of SEQ ID NOS: 5, 7, 9, 11, 13, 15, 17, 19, 21, 23, 25, 27, 29, 31, 33, 35, 37, 39 and 41 by routine recombinant DNA/RNA techniques. A variety of expression vector/host systems may be utilized to contain and express the coding sequence. These include, but are not limited to, microorganisms such as bacteria transformed with recombinant bacteriophage, plasmid, phagemid, or cosmid DNA expression vectors; yeast transformed with yeast expression vectors; insect cell systems infected with viral expression vectors (e.g., baculovirus); plant cell systems transfected with virus expression vectors (e.g., Cauliflower Mosaic Virus, CaMV; Tobacco Mosaic Virus, TMV) or transformed with bacterial expression vectors (e.g., Ti or pBR322 plasmid); or even animal cell systems. Mammalian cells that are useful in recombinant protein productions include, but are not limited to, VERO cells, HeLa cells, Chinese hamster ovary (CHO) cells, COS cells (such as COS-7), WI38, BHK, HepG2, 3T3, RIN, MDCK, A549, PC12, K562 and HEK 293 cells.

Polypeptides of the Invention

In general, the isolated LEC polypeptides of the invention are encoded by the above-described differentially expressed LEC polynucleotides of the invention. The sequences of the LEC polypeptides are provided in Table 1 and 2, associated with their known database accession numbers, where applicable. The isolated polypeptides of the invention include, but are not limited to, a polypeptide comprising: the amino acid sequences set forth as any one of SEQ ID NOS.: 6, 8, 10, 12, 14, 16, 18, 20, 22, 24, 26, 28, 30, 32, 34, 36, 38, 40, 42, 43, 44, 45, and 46 or an amino acid sequence encoded by any one of the nucleotide sequences set forth in SEQ ID NOS.: 6, 8, 10, 12, 14, 16, 18, 20, 22, 24, 26, 28, 30, 32, 34, 36, 38, 40, 42, 43, 44, 45, and 46, or the corresponding full length or mature protein. The invention also provides biologically active or immunologically active variants of any of the amino acid sequences set forth as SEQ ID NOS.: 6, 8, 10, 12, 14, 16, 18, 20, 22, 24, 26, 28, 30, 32, 34, 36, 38, 40, 42, 43, 44, 45, and 46, or the corresponding full length or mature protein suitable variant polypeptides have sequences that are at least about 65%, at least about 70%, at least about 75%, at least about 80%, at least about 85%, 86%, 87%, 88%, 89%, at least about 90%, 91%, 92%, 93%, 94%, preferably at least about 95%, 96%, 97%, more preferably at least about 98%, or most preferably at least about 99% amino acid identity, that retain biological activity. Fragments of the proteins of the present invention which comprise at least 10 contiguous amino acids of a sequence disclosed herein and that are capable of exhibiting a biological activity of the corresponding full length protein are also encompassed by the present invention.

The protein coding sequence is identified in the sequence listing by translation of the disclosed nucleotide sequences. The mature form of such protein may be obtained by expression of a full-length polynucleotide in a suitable mammalian cell or other host cell. The sequence of the mature form of the protein is also determinable from the amino acid sequence of the full-length form. Where proteins of the present invention are membrane bound, soluble forms of the proteins are also provided. In such forms, part or all of the regions causing the proteins to be membrane bound are deleted so that the proteins are capable of being fully secreted from the cell in which it is expressed.

A variety of methodologies known in the art can be utilized to obtain any one of the isolated polypeptides or proteins of the present invention. At the simplest level, the amino acid sequence can be synthesized using commercially available peptide synthesizers. The polypeptides and proteins of the present invention can alternatively be purified from cells which have been altered to express the desired polypeptide or protein. As used herein, a cell is said to be altered to express a desired polypeptide or protein when the cell, through genetic manipulation, is made to produce a polypeptide or protein which it normally does not produce or which the cell normally produces at a lower level. One skilled in the art can readily adapt procedures for introducing and expressing either recombinant or synthetic sequences into eukaryotic or prokaryotic cells in order to generate a cell which produces one of the polypeptides or proteins of the present invention.

A “fragment” of a polypeptide is meant to refer to any portion of the molecule, such as the peptide core, a variant of the peptide core, or an extracellular region of the polypeptide. A “variant” of a polypeptide is meant to refer to a molecule substantially similar in structure and biological activity to either the entire molecule, or to a fragment thereof. Thus, provided that two molecules possess a similar activity, they are considered variants as that term is used herein even if the composition or secondary, tertiary, or quaternary structure of one of the molecules is not identical to that found in the other, or if the sequence of amino acid residues is not identical. An “analogue” of a polypeptide or genetic sequence is meant to refer to a protein or genetic sequence substantially similar in function and structure to the isolated polypeptide or genetic sequence.

It is understood herein that conservative amino acid substitutions can be performed to a purified and isolated polypeptide comprising any one of the sequences of SEQ ID NOS.: 6, 8, 10, 12, 14, 16, 18, 20, 22, 24, 26, 28, 30, 32, 34, 36, 38, 40, 42, 43, 44, 45, and 46 which are likely to result in a polypeptide that retains biological or immunological activity, especially if the number of such substitutions is small. By “conservative amino acid substitution” is meant substitution of an amino acid with an amino acid having a side chain of a similar chemical character. Similar amino acids for making conservative substitutions include those having an acidic side chain (glutamic acid, aspartic acid); a basic side chain (arginine, lysine, histidine); a polar amide side chain (glutamine, asparagine); a hydrophobic, aliphatic side chain (leucine, isoleucine, valine, alanine, glycine); an aromatic side chain (phenylalanine, tryptophan, tyrosine); a small side chain (glycine, alanine, serine, threonine, methionine); or an aliphatic hydroxyl side chain (serine, threonine).

Microarrays

Another aspect of the invention is a composition comprising a plurality of polynucleotide probes for use in detecting gene expression pattern(s) characteristic of particular cell type(s) and for detecting changes in the expression pattern of a particular cell type, e.g., lymphatic endothelial cells. For example, the invention comprehends an array, such as a microarray, comprising polynucleotides having at least 10 contiguous nucleotides selected from the polynucleotide sequences in the sequence listing, or a gene found in any of Tables 1 and 2 herein or Tables 3 and 4 of PCT/US03/06900.

Also contemplated are microarrays comprising polynucleotides having at least 10 contiguous nucleotides selected from the group of SEQ ID NOS: 5, 7, 9, 11, 13, 15, 17, 19, 21, 23, 25, 27, 29, 31, 33, 35, 37, 39 and 41 as well as any one of the genes found Tables 3 and 4 of PCT/US03/06900. Microarrays of the invention comprise at least 3 polynucleotides, wherein each enumerated polynucleotide has a distinct sequence selected from the group consisting of SEQ ID NOS:. 5, 7, 9, 11, 13, 15, 17, 19, 21, 23, 25, 27, 29, 31, 33, 35, 37, 39 and 41 and a genbe found in any of Tables 3 and 4 of PCT/US03/06900. Such microarrays may also have duplicate polynucleotides and additional polynucleotides, e.g., control polynucleotides for use in hybridization-based assays using the microarray. Arrays, including microarrays, having more than three distinct polynucleotides according to the invention, such as at least five, seven, nine, 20, 50 or more such polynucleotides, will be recognized as arrays according to the invention having the capability of yielding subtle distinctions between biological samples such as various endothelial cell types, or of providing a different, and typically greater, level of confidence in the various uses of such arrays, e.g., in screening for particular endothelial cells, in screening for abnormal or diseases cells and tissues, and the like.

The term “microarray” refers to an ordered arrangement of hybridizable array elements. The array elements are arranged so that there are preferably at least three or more different array elements, more preferably at least 100 array elements, and most preferably at least 1,000 array elements, on a solid support. Preferably, the solid support is a 1 cm² substrate surface, bead, paper, nylon or other type of membrane, filter, chip, glass slide, or any other suitable solid support. The hybridization signal from each of the array elements is individually distinguishable. In a preferred embodiment, the array elements comprise polynucleotide probes.

Hybridization means contacting two or more nucleic acids under conditions suitable for base pairing. Hybridization includes interaction between partially or perfectly complementary nucleic acids. Suitable hybridization conditions are well known to those of skill in the art. In certain applications, it is appreciated that lower stringency conditions may be required. Under these conditions, hybridization may occur even though the sequences of the interacting strands are not perfectly complementary, being mismatched at one or more positions. Conditions may be rendered less stringent by adjusting conditions in accordance with the knowledge in the art, e.g., increasing salt concentration and/or decreasing temperature. Suitable hybridization conditions are those conditions that allow the detection of gene expression from identifiable expression units such as genes. Preferred hybridization conditions are stringent hybridization conditions, such as hybridization at 42° C. in a solution (i.e., a hybridization solution) comprising 50% formamide, 1% SDS, 1 M NaCl, 10% dextran sulfate, and washing for 30 minutes at 65° C. in a wash solution comprising 1×SSC and 0.1% SDS. It is understood in the art that conditions of equivalent stringency can be achieved through variation of temperature and buffer, or salt concentration, as described in Ausubel, et al. (Eds.), Protocols in Molecular Biology, John Wiley & Sons (1994), pp. 6.0.3 to 6.4.10. Modifications in hybridization conditions can be empirically determined or precisely calculated based on the length and the percentage of guanosine/cytosine (GC) base pairing of the probe. The hybridization conditions can be calculated as described in Sambrook, et al., (Eds.), Molecular Cloning: A Laboratory Manual, Cold Spring Harbor Laboratory Press: Cold Spring Harbor, N.Y. (2d. Ed.; 1989), pp. 9.47 to 9.51.

One method of using probes and primers of the invention is in the detection of gene expression in human cells. Normally, the target will be expressed RNAs, although genomic DNA or a cDNA library may be screened. By varying the stringency of hybridization and the target binding site (i.e., the sequence of the probe, corresponding to a subset of one of the sequences set forth at SEQ ID NOS: 5, 7, 9, 11, 13, 15, 17, 19, 21, 23, 25, 27, 29, 31, 33, 35, 37, 39 and 41 or a gene found in any of Tables 3 and 4 of PCT/US03/06900), different degrees of homology are expected to result in hybridization.

The microarray can be used for large-scale genetic or gene expression analysis of a large number of target polynucleotides. The microarray can also be used in the diagnosis of diseases and in the monitoring of treatments. Further, the microarray can be employed to investigate an individual's predisposition to a disease. Furthermore, the microarray can be employed to investigate cellular responses to infection, drug treatment, and the like.

The nucleic acid probes can be genomic DNA or cDNA or mRNA polynucleotides or oligonucleotides, or any RNA-like or DNA-like material, such as peptide nucleic acids, branched DNAs, and the like. The probes can be sense or antisense nucleotide probes. Where target polynucleotides are double-stranded, the probes may be either sense or antisense strands. Where the target polynucleotides are single-stranded, the probes are complementary single strands. In one embodiment, the probes are cDNAs. The size of the DNA sequence of interest may vary and is preferably from 100 to 10,000 nucleotides, more preferably from 150 to 3,500 nucleotides.

The probes can be prepared using a variety of synthetic or enzymatic techniques, which are well known in the art. The probes can be synthesized, in whole or in part, using chemical methods well known in the art (Caruthers et al., Nucleic Acids Res., Symp. Ser., 215-233, 1980).

Pharmaceutical Formulations and Routes of Administration

A protein of the present invention (from whatever source derived, such as from recombinant and non-recombinant sources) may be administered to a patient in need, by itself, or in pharmaceutical compositions where it is mixed with suitable carriers, diluents, adjuvants or excipients at doses to treat or ameliorate a variety of disorders. Such a composition may also contain (in addition to protein and a carrier) diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration. The pharmaceutical composition of the invention may also contain cytokines, chemokines, lymphokines, growth factors, or other hematopoietic factors such as a PDGF, a VEGF (particularly a VEGF-C or a VEGF-D), VEGFR-3 (including soluble VEGFR-3 peptides comprising an extracellular domain), M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IFN, TNF0, TNF1, TNF2, G-CSF, Meg-CSF, thrombopoietin, stem cell factor, and erythropoietin. Various forms of these polypeptides are contemplated as well, such as isolated holoproteins, subunits, fragments (e.g., soluble fragments), and peptide fusions. The pharmaceutical composition may further contain other agents which either enhance the activity of the protein or complement its activity or use in treatment. Such additional factors and/or agents may be included in the pharmaceutical composition to produce a synergistic effect with a protein of the invention, or to minimize side effects. Conversely, a protein of the present invention may be included in formulations of the particular cytokine, lymphokine, other hematopoietic factor, thrombolytic or anti-thrombotic factor, or anti-inflammatory agent to minimize side effects of the cytokine, lymphokine, other hematopoietic factor, thrombolytic or anti-thrombotic factor, or anti-inflammatory agent. A protein of the present invention may be active in multimers (e.g., heterodimers or homodimers) or complexes with itself or other proteins. As a result, pharmaceutical compositions of the invention may comprise a protein of the invention in such multimeric or complexed form.

Techniques for formulation and administration of the compounds of the instant application may be found in “Remington's Pharmaceutical Sciences,” Mack Publishing Co., Easton, Pa., latest edition. A therapeutically effective dose further refers to that amount of the compound sufficient to result in amelioration of symptoms, e.g., treatment, healing, prevention or amelioration of the relevant medical condition, or an increase in rate of beneficial change, healing, prevention or amelioration of such conditions. When applied to an individual active ingredient, administered alone, a therapeutically effective dose refers to that ingredient alone. When applied to a combination, a therapeutically effective dose refers to combined amounts of the active ingredients that result in the therapeutic effect, whether administered in combination, serially or simultaneously.

In practicing methods of treatment or use of the present invention, a therapeutically effective amount of protein of the present invention is administered to a mammal having a condition or disorder to be treated. Protein of the present invention may be administered in accordance with the method of the invention either alone or in combination with other therapies such as treatments employing cytokines, lymphokines or other hematopoietic factors. When co-administered with one or more cytokines, lymphokines or other hematopoietic factors, a protein of the invention may be administered either simultaneously with the cytokine(s), lymphokine(s), other hematopoietic factor(s), thrombolytic or anti-thrombotic factors, or sequentially. If administered sequentially, the attending physician will decide on the appropriate sequence of administering a protein of the invention in combination with a cytokine(s), lymphokine(s), other hematopoietic factor(s), thrombolytic or anti-thrombotic factors.

Routes of Administration

Suitable routes of administration may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. Administration of protein of the present invention used in the pharmaceutical composition or to practice the method of the present invention is carried out in a variety of conventional ways, such as oral ingestion, inhalation, topical application or cutaneous, subcutaneous, intraperitoneal, parenteral or intravenous injection. Intravenous administration to a mammal, such as a human patient, is preferred. Alternately, one may administer the compound in a local rather than systemic manner, for example, via injection of the compound at the site of intended action.

Compositions/Formulations

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. These pharmaceutical compositions may be manufactured in a manner that is itself known, e.g., by means of conventional mixing, dissolving, granulating, dragee-making, levigating, emulsifying, encapsulating, entrapping or lyophilizing processes. Proper formulation is dependent upon the route of administration chosen. When a therapeutically effective amount of protein of the present invention is administered orally, protein of the present invention will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% protein of the present invention, and preferably from about 25 to 90% protein of the present invention. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of protein of the present invention, and preferably from about 1 to 50% protein of the present invention.

When a therapeutically effective amount of protein of the present invention is administered by intravenous, cutaneous or subcutaneous injection, protein of the present invention will be in the form of a pyrogen-free, parenterally acceptable aqueous solution. The preparation of such parenterally acceptable protein solutions, having due regard to pH, isotonicity, stability, and the like, is within the skill in the art. A preferred pharmaceutical composition for intravenous, cutaneous, or subcutaneous injection should contain, in addition to protein of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art. For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by combination with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of PD diluted 1:1 with a 5% dextrose-in-water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g., polyvinyl pyrrolidone; and other sugars or polysaccharides may substitute for dextrose. Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compound over a time period of a few weeks up to over 100 days. Depending on the chemical nature and the biological stability of the therapeutic reagent, additional strategies for protein stabilization may be employed.

The pharmaceutical compositions also may comprise suitable solid or gel-phase carriers or excipients. Examples of such carriers or excipients include but are not limited to calcium carbonate, calcium phosphate, various sugars, starches, cellulose derivatives, gelatin, and polymers such as polyethylene glycols. Many of the proteinase-inhibiting compounds of the invention may be provided as salts with pharmaceutically compatible counterions. Such pharmaceutically acceptable base addition salts are those salts which retain the biological effectiveness and properties of the free acids and which are obtained by reaction with inorganic or organic bases such as sodium hydroxide, magnesium hydroxide, ammonia, trialkylamine, dialkylamine, monoalkylamine, dibasic amino acids, sodium acetate, potassium benzoate, triethanol amine, and the like.

The pharmaceutical compositions of the invention may be in the form of a complex of a protein(s) of the present invention along with protein or peptide antigens. The pharmaceutical compositions of the invention may be in the form of a liposome in which protein of the present invention is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323, each of which is incorporated herein by reference.

The amount of protein of the invention in the pharmaceutical composition will depend upon the nature and severity of the condition being treated, and on the nature of prior treatments which the patient has undergone. Ultimately, the attending physician will decide the amount of protein of the present invention with which to treat each individual patient. Initially, the attending physician will administer low doses of protein of the present invention and observe the patient's response. Larger doses of protein of the present invention may be administered until the optimal therapeutic effect is obtained for the patient, and at that point the dosage is not increased further. It is contemplated that the various pharmaceutical compositions used to practice the methods of the invention should contain about 0.01 μg to about 100 mg (preferably about 0.1 μg to about 10 mg, more preferably about 0.1 μg to about 1 mg) of protein of the invention per kg body weight. When administered, the therapeutic composition for use in this invention is in a pyrogen-free, physiologically acceptable form. Therapeutically useful agents other than a protein of the invention which may also optionally be included in the composition as described above, may alternatively or additionally, be administered simultaneously or sequentially with the composition in the methods of the invention.

Polynucleotides of the present invention can also be used for gene therapy. Such polynucleotides can be introduced either in vivo or ex vivo into cells for expression in a mammalian subject. Polynucleotides of the invention may also be administered by other known methods for introduction of nucleic acid into a cell or organism (including, without limitation, in the form of viral vectors or naked DNA). Cells may also be cultured ex vivo in the presence of proteins of the present invention in order to proliferate or to produce a desired effect on or activity in such cells. Treated cells can then be introduced in vivo for therapeutic purposes.

Effective Dosage

Pharmaceutical compositions suitable for use in the present invention include compositions wherein the active ingredients are contained in an effective amount to achieve an intended purpose. More specifically, a therapeutically effective amount means an amount effective to prevent development of, or to alleviate the existing symptoms of, the subject being treated. Suitable properties that may be used in determining effective dosages include measurements of LEC and/or BEC growth stimulation or inhibition, rates or extent of cell differentiation into LECs and/or BECs, tendencies of cell expression patterns to shift towards or away from LEC- or BEC-specific expression patterns, and the like. Determination of the effective amounts is well within the capability of those skilled in the art, especially in light of the detailed disclosure provided herein. For any compound used in a method of the invention, the therapeutically effective dose can be estimated initially from cell culture assays. For example, for inhibitory methods, a dose can be formulated in animal models to achieve a circulating concentration range that includes the IC₅₀ as determined in cell culture (i.e., the concentration of the test compound which achieves a half-maximal inhibitory concentration). Such information can be used to more accurately determine useful doses in humans.

A therapeutically effective dose refers to that amount of the compound that results in amelioration of symptoms or, in the case of life-threatening conditions, a prolongation of survival in a patient. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds which exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. See, e.g., Fingl et al., 1975, in “The Pharmacological Basis of Therapeutics”, Ch. 1 p. 1.

The amount of composition administered will, of course, be dependent on the subject being treated, on the subject's weight, the severity of the affliction, the manner of administration and the judgment of the prescribing physician.

Packaging

The compositions may, if desired, be presented in a pack or dispenser device which may contain one or more unit dosage forms containing the active ingredient. The pack may, for example, comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

In addition, the invention comprehends a use of such a composition to manufacture a medicament for the treatment of a cell or an organism, such as a human patient, having a hyperproliferative or hypoproliferative disorder of a LEC and/or BEC, such as lymphedema, lymphangioma, lymphangiomyeloma, lymphangiomatosis, lymphangiectasis, lymphosarcoma, or lymphangiosclerosis, comprising administering an effective amount, or dose, of a composition according to the invention to the cell or organism. Suitable compositions include, but are not limited to, any polynucleotide according to the invention (e.g., an antisense polynucleotide), any polypeptide according to the invention, an antibody specifically recognizing a polynucleotide or polypeptide according to the invention, a small molecule compound effective in modulating the expression of a polynucleotide according to the invention, and the like. Also contemplated are uses of compositions according to the invention for the manufacture of a medicament to ameliorate a symptom associated with a LEC- or BEC-associated disease or disorder.

Antibodies

Antibodies are useful for modulating the polypeptides of the invention due to the ability to easily generate antibodies with relative specificity, and due to the continued improvements in technologies for adopting antibodies to human therapy. Thus, the invention contemplates use of antibodies (e.g., monoclonal and polyclonal antibodies, single chain antibodies, chimeric antibodies, bifunctional/bispecific antibodies, humanized antibodies, human antibodies, domain antibodies and complementary determining region (CDR)-grafted antibodies, including compounds which include CDR sequences which specifically recognize a polypeptide of the invention), specific for polypeptides of interest to the invention. Preferred antibodies are human antibodies, such as those produced in transgenic animals, which are produced and identified according to methods described in WO 93/11236, published Jun. 20, 1993, which is incorporated herein by reference in its entirety. Antibody fragments, including Fab, Fab′, F(ab′)2, and Fv, are also provided by the invention. The term “specific for,” when used to describe antibodies of the invention, indicates that the variable regions of the antibodies of the invention recognize and bind the polypeptide of interest at a detectably different, and greater, level that bind to other substances (i.e., able to distinguish the polypeptides of interest from other known polypeptides of the same family, by virtue of measurable differences in binding affinity, despite the possible existence of localized sequence identity, homology, or similarity between family members). It will be understood that specific antibodies may also interact with other proteins (for example, S. aureus protein A or other antibodies in ELISA techniques) through interactions with sequences outside the variable region of the antibodies, and in particular, in the constant region of the molecule. Screening assays to determine binding specificity of an antibody of the invention are well known and routinely practiced in the art. For a comprehensive discussion of such assays, see Harlow et al. (Eds.), Antibodies A Laboratory Manual; Cold Spring Harbor Laboratory; Cold Spring Harbor, N.Y. (1988), Chapter 6.

Non-human antibodies may be humanized by any method known in the art. A preferred “humanized antibody” has a human constant region, while the variable region, or at least a complementarity-determining region (CDR), of the antibody is derived from a non-human species. Methods for humanizing non-human antibodies are well known in the art. See U.S. Pat. Nos. 5,585,089, and 5,693,762. Generally, a humanized antibody has one or more amino acid residues introduced into its framework region from a source which is non-human. Humanization can be performed, for example, using methods described in Jones et al (Nature 321: 522-525, 1986), Riechmann et al., (Nature, 332: 323-327, 1988) and Verhoeyen et al. [Science 239:1534-1536, (1988)], by substituting at least a portion of a rodent CDR for the corresponding regions of a human antibody. Numerous techniques for preparing engineered antibodies are described, e.g., in Owens and Young, (J. Immunol. Meth., 168:149-165, 1994). Further changes can then be introduced into the antibody framework to modulate affinity or immunogenicity.

The invention further provides a hybridoma that produces an antibody according to the invention. Antibodies of the invention are useful for detection and/or purification of the polypeptides of the invention.

Polypeptides and/or polynucleotides of the invention may also be used to immunize animals to obtain polyclonal and monoclonal antibodies which specifically react with the polypeptide. Such antibodies may be obtained using either the entire polypeptide or fragments thereof as an immunogen. The peptide immunogens additionally may contain a cysteine residue at the carboxyl terminus, and may be conjugated to a hapten such as keyhole limpet hemocyanin (KLH). Methods for synthesizing such peptides are known in the art, for example, as taught in R. P. Merrifield, J. Amer. Chem. Soc. 85:2149-2154 (1963); J. L. Krstenansky, et al., FEBS Lett. 211: 10 (1987). Monoclonal antibodies binding to the protein of the invention may be useful diagnostic agents for the immunodetection of the polypeptide. Neutralizing monoclonal antibodies binding to the polypeptide may also be useful therapeutics for both conditions associated with the polypeptide and also in the treatment of some forms of cancer where abnormal expression of the polypeptide is involved. In the case of cancerous cells or leukemic cells, neutralizing monoclonal antibodies against the polypeptide are useful in detecting and preventing the metastatic spread of the cancerous cells mediated by the polypeptide. In general, techniques for preparing polyclonal and monoclonal antibodies as well as hybridomas capable of producing the desired antibody are well known in the art (Campbell, A. M., Monoclonal Antibodies Technology: Laboratory Techniques in Biochemistry and Molecular Biology, Elsevier Science Publishers, Amsterdam, The Netherlands (1984); St. Groth et al., J. Immunol. 35:1-21 (1990); Kohler and Milstein, Nature 256:495-497 (1975), the trioma technique, the human B-cell hybridoma technique (Kozbor et al., Immunology Today 4:72, 1983); Cole et al., in Monoclonal Antibodies and Cancer Therapy, Alan R. Liss, Inc. (1985), pp. 77-96).

Any animal (mouse, rabbit, and the like) which is known to produce antibodies can be immunized with a peptide or polypeptide of the invention. Methods for immunization are well known in the art. Such methods include subcutaneous or intraperitoneal injection of the polypeptide. One skilled in the art will recognize that the amount of the polypeptide encoded by an ORF of the invention used for immunization will vary based on the animal which is immunized, the antigenicity of the peptide and the site of injection. The protein that is used as an immunogen may be modified or administered in an adjuvant in order to increase the protein's antigenicity. Methods of increasing the antigenicity of a protein are well known in the art and include, but are not limited to, coupling the antigen with a heterologous protein (such as a globulin or (β-galactosidase) or through the inclusion of an adjuvant during immunization.

For monoclonal antibodies, spleen cells from the immunized animals are removed, fused with myeloma cells, such as SP2/0-Ag14 myeloma cells, and allowed to become monoclonal-antibody-producing hybridoma cells. Any one of a number of methods well known in the art can be used to identify the hybridoma cell which produces an antibody with the desired characteristics. These include screening the hybridomas with an ELISA assay, Western blot analysis, or radioimmunoassay (Lutz et al., Exp. Cell Research. 175:109-124, 1988). Hybridomas secreting the desired antibodies are cloned and the class and subclass is determined using procedures known in the art (Campbell, A. M., Monoclonal Antibody Technology: Laboratory Techniques in Biochemistry and Molecular Biology, Elsevier Science Publishers, Amsterdam, The Netherlands (1984)). Techniques described for the production of single-chain antibodies (U.S. Pat. No. 4,946,778) can be adapted to produce single-chain antibodies to polypeptide of the invention.

For polyclonal antibodies, antibody-containing antiserum is isolated from the immunized animal and is screened for the presence of antibodies with the desired specificity using one of the above-described procedures. The invention further provides the above-described antibodies in detectably labeled form. Antibodies can be detectably labeled through the use of radioisotopes, affinity labels (such as biotin, avidin, and the like), enzymatic labels (such as horseradish peroxidase, alkaline phosphatase, and the like) fluorescent labels (such as FITC or rhodamine, and the like), paramagnetic atoms, and the like. Procedures for accomplishing such labeling are well-known in the art; for example, see Stemberger, et al., J. Histochem. Cytochem. 18:315, 1970; Bayer, et al., Meth. Enzym. 62:308, 1979; Engval, et al., Immunol. 109:129, 1972; and Goding, J. Immunol. Meth. 13:215, 1976.

The labeled antibodies of the present invention can be used for in vitro, in vivo, and in situ assays to identify cells or tissues in which a fragment of the polypeptide of interest is expressed. The antibodies may also be used directly in therapies or other diagnostics. The present invention further provides the above-described antibodies immobilized on a solid support. Examples of such solid supports include plastics such as polycarbonate, complex carbohydrates such as agarose and sepharose, and acrylic resins such as polyacrylamide and latex beads. Techniques for coupling antibodies to such solid supports are well known in the art (Weir, D. M. et al., “Handbook of Experimental Immunology” 4th Ed., Blackwell Scientific Publications, Oxford, England, Chapter 10 (1986); Jacoby, W. D. et al., Meth. Enzym. 34 Academic Press, N.Y. (1974)). The immobilized antibodies of the present invention can be used for in vitro, in vivo, and in situ assays as well as for immuno-affinity purification of the proteins of the present invention.

Computer-Readable Sequences

In one application of this embodiment, a nucleotide sequence of the present invention can be recorded on computer-readable media. As used herein, “computer-readable media” refers to any medium which can be read and accessed directly by a computer. Such media include, but are not limited to, magnetic storage media, such as floppy discs, hard disc storage medium, and magnetic tape; optical storage media such as CD-ROM; electrical storage media such as RAM and ROM; and hybrids of these categories such as magnetic/optical storage media. A skilled artisan can readily appreciate how any of the presently known computer-readable media can be used to create a manufacture comprising computer-readable medium having recorded thereon a nucleotide sequence of the present invention. As used herein, “recorded” refers to a process for storing information on computer-readable medium. A skilled artisan can readily adopt any of the presently known methods for recording information on a computer-readable medium to generate manufactures comprising the nucleotide sequence information of the present invention.

A variety of data storage structures are available to a skilled artisan for creating a computer-readable medium having recorded thereon a nucleotide sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. A skilled artisan can readily adapt any number of data processor structuring formats (e.g., text file or database) in order to obtain computer readable medium having recorded thereon the nucleotide sequence information of the present invention. By providing the nucleotide sequence of any of SEQ ID NOS: 5, 7, 9, 11, 13, 15, 17, 19, 21, 23, 25, 27, 29, 31, 33, 35, 37, 39 and 41 or a representative fragment thereof, or a nucleotide sequence at least 99.9% identical to any of SEQ ID NOS: 5, 7, 9, 11, 13, 15, 17, 19, 21, 23, 25, 27, 29, 31, 33, 35, 37, 39 and 41 in computer-readable form, a skilled artisan can routinely access the sequence information for a variety of purposes. Computer software is publicly available which allows a skilled artisan to access sequence information provided in a computer-readable medium. The examples which follow demonstrate how software which implements the BLAST (Altschul et al., J. Mol. Biol. 215:403-410, 1990) and BLAZE (Brutlag et al., Comp. Chem. 17:203-207, 1993) search algorithms on a Sybase system is used to identify open reading frames (ORFs) within a nucleic acid sequence. Such ORFs may be protein-encoding fragments and may be useful in producing commercially important proteins such as enzymes used in fermentation reactions and in the production of commercially useful metabolites.

As used herein, “a computer-based system” refers to the hardware means, software means, and data storage means used to analyze the nucleotide sequence information of the present invention. The minimum hardware means of the computer-based systems of the present invention comprises a central processing unit (CPU), input means, output means, and data storage means. A skilled artisan can readily appreciate that any one of the currently available computer-based systems are suitable for use in the invention. As stated above, the computer-based systems of the present invention comprise a data storage means having stored therein a nucleotide sequence of the present invention and the necessary hardware means and software means for supporting and implementing a search means. As used herein, “data storage means” refers to memory which can store nucleotide sequence information of the present invention, or a memory access means, which can access manufactures having recorded thereon the nucleotide sequence information of the present invention.

As used herein, “search means” refers to one or more programs which are implemented on the computer-based system to compare a target sequence or target structural motif with the sequence information stored within the data storage means. Search means are used to identify fragments or regions of a known sequence which match a particular target sequence or target motif. A variety of known algorithms are disclosed publicly and a variety of commercially available software for conducting search means are and can be used in the computer-based systems of the present invention. Examples of such software includes, but is not limited to, MacPattern (EMBL), BLASTN and BLASTA (NPOLYPEPTIDEIA). A skilled artisan can readily recognize that any one of the available algorithms or implementing software packages for conducting homology searches can be adapted for use in the present computer-based systems. As used herein, a “target sequence” can be any nucleic acid or amino acid sequence of six or more nucleotides or two or more amino acids. A skilled artisan can readily recognize that the longer a target sequence is, the less likely a target sequence will be present as a random occurrence in the database. The most preferred sequence length of a target sequence is from about 10 to 100 amino acids or from about 30 to 300 nucleotide residues. However, it is well recognized that searches for commercially important fragments, such as sequence fragments involved in gene expression and protein processing, may be of shorter length.

As used herein, “a target structural motif,” or “target motif,” refers to any rationally selected sequence or combination of sequences in which the sequence(s) are chosen based on a three-dimensional configuration which is formed upon the folding of the target motif. There are a variety of target motifs known in the art. Protein target motifs include, but are not limited to, enzyme active sites and signal sequences. Nucleic acid target motifs include, but are not limited to, promoter sequences, hairpin structures and inducible expression elements (protein binding sequences).

Diagnostic Assays and Kits

The present invention further provides diagnostic assays, and related kits, for hyper- and/or hypo-proliferative disorders or diseases of endothelial cells such as LECs or BECs. These assays comprise methods to identify the presence or expression of one of the ORFs of the present invention, or homolog thereof, in a test sample, using a nucleic acid probe or an antibody according to the invention.

In general, methods for detecting a polynucleotide of the invention can comprise contacting a sample with a compound that binds to and forms a complex with, the polynucleotide for a period sufficient to form the complex, and detecting the complex, so that if a complex is detected, a polynucleotide of the invention is detected in the sample.

Such methods can also comprise contacting a sample under stringent hybridization conditions with nucleic acid primers that anneal to a polynucleotide of the invention under such conditions, and amplifying annealed polynucleotides, so that if a polynucleotide is amplified, a polynucleotide of the invention is detected in the sample.

In general, methods for detecting a polypeptide of the invention can comprise contacting a sample with a compound that binds to and forms a complex with the polypeptide for a period sufficient to form the complex, and detecting the complex, so that if a complex is detected, a polypeptide of the invention is detected in the sample. In detail, such methods comprise incubating a test sample with one or more of the antibodies or one or more of the nucleic acid probes of the invention and assaying for binding of the nucleic acid probes or antibodies to components within the test sample.

Conditions for incubating a nucleic acid probe or antibody with a test sample vary. Incubation conditions depend on the format employed in the assay, the detection methods employed, and the type and nature of the nucleic acid probe or antibody used in the assay. One skilled in the art will recognize that any one of the commonly available hybridization, amplification or immunological assay formats can readily be adapted to employ the nucleic acid probes or antibodies of the present invention. Examples of such assays can be found in Chard, T., An Introduction to Radioimmunoassay and Related Techniques, Elsevier Science Publishers, Amsterdam, The Netherlands (1986); Bullock, G. R et al., Techniques in Immunocytochemistry, Academic Press, Orlando, Fla. Vol. 1 (1982), Vol. 2 (1983), Vol. 3 (1985); Tijssen, P., Practice and Theory of immunoassays: Laboratory Techniques in Biochemistry and Molecular Biology Elsevier Science Publishers, Amsterdam, The Netherlands (1985). The test samples of the present invention include cells, protein or membrane extracts of cells, or biological fluids such as sputum, blood, serum, plasma, or urine. The test sample used in the above-described method will vary based on the assay format, nature of the detection method and the tissues, and cells or extracts used as the sample to be assayed. Methods for preparing protein extracts or membrane extracts of cells are well known in the art and can be readily be adapted in order to obtain a sample which is compatible with the system utilized.

In another embodiment of the present invention, kits are provided which contain the necessary reagents to carry out the assays of the present invention. In one embodiment, the invention provides a compartment kit to receive, in close confinement, one or more containers which comprises: (a) a first container comprising one of the probes or antibodies of the present invention; and (b) one or more other containers comprising one or more of the following: wash reagents, reagents capable of detecting presence of a bound probe or antibody.

In detail, a compartment kit includes any kit in which reagents are contained in separate containers. Such containers include small glass containers, plastic containers or strips of plastic or paper. Such containers allows one to efficiently transfer reagents from one compartment to another compartment such that the samples and reagents are not cross-contaminated, and the agents or solutions of each container can be added in a quantitative fashion from one compartment to another. Such containers will include a container which will accept the test sample, a container which contains the antibody or antibodies used in the assay, containers which contain wash reagents (such as phosphate-buffered saline, Tris buffers, and the like), and containers which contain the reagents used to detect the bound antibody or probe. Types of detection reagents include labeled nucleic acid probes, labeled secondary antibodies, or in the alternative, if the primary antibody is labeled, the enzymatic, or antibody binding reagents which are capable of reacting with the labeled antibody. One skilled in the art will readily recognize that the disclosed probes and antibodies of the present invention can be readily incorporated into one of the established kit formats which are well known in the art.

### EXAMPLES

Each reference cited in the methods described below is incorporated by reference herein. Methods used in the examples are as follows:

Antibodies

Monoclonal antibodies against human VEGFR-3 (clone 2E11D11; see International Patent Application No. PCT/US02/22164, published as WO 03/006104), PAL-E (Monosan), CD31 (Dako), N-cadherin, VE-cadherin, β-catenin and plakoglobin and polyclonal rabbit anti-human podoplanin were used (Breiteneder-Geleff, S., et al., Am. J. Pathol. 154:385-394, 1999). Mouse anti-human integrin α9 was provided by Dr. Dean Sheppard (University of California at San Francisco, San Francisco) and Dr. Curzio Rüegg (University of Lausanne Medical School, Lausanne, Switzerland). The fluorocbrome-conjugated secondary antibodies were obtained from Jackson Immunoresearch.

Cell Culture and Transfection

Human amniotic epithelial cells are cultured in Med199 medium in the presence of 5% fetal calf serum. Human dermal microvascular endothelial cells are obtained from PromoCell (Heidelberg, Germany). Anti-Podoplanin antibodies, MACS colloidal super-paramagnetic MicroBeads conjugated to goat anti-rabbit IgG antibodies (Miltenyi Biotech, Bergisch Gladbach, Germany), LD and MS separation columns and Midi/MiniMACS separators (Miltenyi Biotech) are used for cell sorting according to the instructions of the manufacturer. The isolated cells are cultured on fibronectin-coated (10 μg/ml, Sigma, St. Louis, Mo.) plates as described (Mäkinen, T., et al., EMBO J 20:4762-4773, 2001).

Immunofluorescence and Immunohistochemistry

To carry out immunofluorescence on isolated epithelial cells, the cells are cultured on coverslips, fixed with 4% paraformaldehyde, permeabilized with 0.1% Triton-X100 in phosphate-buffered saline (PBS) and stained with primary antibodies. For integrin α9, staining live cells were incubated with the antibody for 15 minutes on ice before fixation. The cells were further stained with FITC- or TRITC-conjugated secondary antibodies. F-actin is stained using TexasRed-conjugated phalloidin (Molecular Probes, Eugene, Oreg.). Cells are counterstained with Hoechst 33258 fluorochrome (Sigma) and viewed using a Zeiss Axioplan 2 fluorescent microscope.

Normal human skin obtained after surgical removal is embedded in Tissue-Tek® (Sakura, The Netherlands), frozen and sectioned. The sections (6 μm) are fixed in cold acetone for 10 minutes and stained with the primary antibodies followed by peroxidase staining using Vectastain Elite ABC kit (Vector Laboratories, Burlingame, Calif.) and 3-amino-9-ethyl carbazole (Sigma, St. Louis, Mo.).

RNA Isolation Northern Blotting and Microarray Analyses

Total RNA was isolated and DNAseI treated in RNeasy columns (Qiagen, Valencia, Calif.). ³²P-labeled probes for hybridization with the Atlas filters (Clontech) were prepared using 2-5 μg of total RNA according to the manufacturer's instructions with the exception that the probe was purified using Nick-25 columns (Pharmacia Biotech, Uppsala, Sweden). Following hybridizations and washes, the membranes were analyzed using a Fuji BAS 100 phosphoimager. For the Affymetrix® M analysis, 2-5 RNA samples isolated from LEC and BEC cell cultures were hybridized separately using RNA extracted from four lots of cells isolated from different individuals. For the Affymetrix® expression analysis, 5 μg of total RNA was used for the synthesis of double-stranded cDNA using Custom SuperScript ds-cDNA Synthesis Kit (Invitrogen, Carlsbad, Calif.). Biotin-labeled cRNA was then prepared using the Enzo BioArrayTMHighYieldTMRNA Transcript Labeling Kit (Affymetrix, Santa Clara, Calif.), and the unincorporated nucleotides were removed using RNeasy columns (Qiagen, Valencia, Calif.). The hybridization, washing and staining of Human Genome U133A microarrays were done according to the instructions of the manufacturer (Affymetrix, GeneChip Expression Analysis Technical Manual). The probe arrays were scanned at 570 nm using an Agilent GeneArray® Scanner and the readings from the quantitative scanning were analyzed by the Affymetrix® Microarray Suite version 5.0 and Data Mining Tool version 3.0. For the comparison analyses, the hybridization intensities were calculated using a global scaling intensity of 100.

The differentially expressed sequences were used for searching EST contigs in the GenBank database of the National Center for Biotechnology Information and the National Library of Medicine. (NCBI/NLM), and open reading frames were predicted using the orf finder software available at NCBI/NLM. The SOSUI system was used for prediction of transmembrane helices and signal sequences from the protein sequences, and other protein domain architectures were analyzed using Pfam (Protein families database of alignments and HMMs).

It is expected that the amino acid sequence of a given polypeptide, whether obtained from the Swissprot database, which provides the sequences for typical Pfam analyses, or a Genbank sequence, would be substantially, if not exactly, identical over a typically predominating overlap between those sequences. Moreover, the polypeptides characterized by either of the sequences would be expected to have the same or substantially similar biological activities. Differences in the two sequences may arise due to the fact that some polypeptides identified and used in the Pfam analyses may contain domains designated as “low complexity” or “coiled coil” that are not included in the sequence of a final polypeptide product, as provided by Genbank. Additionally, the Genbank polynucleotide sequences provided herein, which encode the polypeptides disclosed herein, are expected to correspond to polynucleotides encoding the polypeptides utilized in the Pfam analysis.

### Example 1

### IDENTIFICATION OF CELL SURFACE PROTEINS EXPRESSED DIFFERENTIALLY IN LEC

Lymphatic endothelial cells (LEC) were isolated from cultures of human dermal microvascular endothelial cells using magnetic microbeads and antibodies against the lymphatic endothelial cell surface marker podoplanin (Breiteneder-Geleff, et al., Am. J. Pathol. 154:385-394 (1999); Mäkinen, et al., EMBO J. 20:4762-4773 (2001)), as described in PCT/US03/06900. The purities of the isolated LEC populations were confirmed to be over 99% as assessed by immunofluorescence using antibodies against VEGFR-3 or podoplanin. The isolated ells were cultured for a couple of passages, and RNA was extracted from the cultures and used for hybridization with oligonucleotide microarrays containing sequences from about 12,000 known genes, ie., approximately ⅓ of the total number of all predicted human transcripts.

PCT/US03/06900, which examined LEC specific genes based on the 95Av2 and 9513-E arrays, identified approximately 165 LEC specific genes in several different protein families, such as adhesion molecules, cytoskeletal proteins, extracellular matrix proteins transcription factors and growth factors.

Experiments were performed to characterize LEC gene expression profiles revealed by the U133A microarray. The Human Genome U133 (HG-U133) Set, consisting of two GeneChip® arrays, contains almost 45,000 probe sets representing more than 39,000 human gene transcripts derived from approximately 33,000 well-substantiated human genes. This set design uses sequences selected from GenBank®, dbEST, and RefSeq. The HG-U133A Array includes representation of the RefSeq database sequences and probe sets. The HG-U133B Array contains primarily probe sets representing EST clusters. The U133A array utilized herein provides a greater sampling of human genes and ESTs (approximately 20,0000 more genes) than the arrays described previously in PCT/US03/06900.

Differentially expressed genes were delineated from the U133A array if their signal ratio was over 1.1 and p-value below 0.05, resulting in over 600 genes found to be differentially expressed between LECs and BECs.

LEC-specific genes were further analyzed using a subtraction library involving LEC and BEC genes. To construct the library, total RNA was isolated as previously described and 5 μg of total RNA was pre-amplified using a SMART™ PCR cDNA synthesis kit (BD Biosciences Clontech). After RsaI-digestion, PCR-Select cDNA subtraction was carried out in both directions, resulting in selective amplification of differentially expressed sequences, and subtracted LEC and BEC cDNA libraries were prepared (BD Biosciences Clontech). Subtractive hybridization was performed with a 1 (tester): 30 (driver) ratio in both directions and subtracted cDNA pools were amplified by PCR. Forty nanograms of the purified PCR-amplified product were cloned into the pAtlas vector (PUC-based vector) for the construction of subtracted libraries, although a number of other vectors could be used in the construction, as would be known in the art.

Differential screening of the subtracted LEC-specific library was carried out as described in the PCR-Select Differential Screening Kit User Manual (BD Biosciences, Clontech). The LEC-specific subtracted library was plated and individual bacterial clones were picked and grown. After DNA extraction, the inserts were amplified by PCR and used for sequencing. An aliquot of each PCR-amplified insert was also arrayed onto a nylon membrane and used for hybridization with ³²P-labeled cDNA probes. The results from the hybridizations with subtracted LEC-specific (tester) and subtracted BEC-specific (driver) cDNA probes were used for the differential expression analyses.

BLAST (The Basic Local Alignment Search Tool) was used to compare the sequences against nucleotide, protein and EST sequence databases. For unknown sequences, EST contigs were searched and open reading frames were predicted using ORF finder. Protein domain architectures were analyzed using Pfam (Protein families database of alignments and HMMs) and Smart (Simple Modular Architecture Research Tool).

The nucleotide sequences of clones that were differentially expressed in LECs versus BECs were analyzed in the manner described above. Several of the EST or unknown gene fragments detected in the first screen have been investigated further to determine their sequence similarities to known gene sequences and to identify any open reading frames and functional domain similarities.

Several of the LEC-specific genes have been found to correspond to KIAA gene sequences, which are large nucleotide EST clones encoding unknown human proteins. (Kazusa DNA Research Institute, 1532-3, Yana Kisarazu, Chiba, 292-0812, Japan). These LEC-specific genes were further analyzed in several available databases to determine the existence of species homologs and the percent similarity in these homologs and also to reveal amino acid sequences that demonstrate similarity to conserved protein domains.

Analyses of the LEC clone sequences were performed using the HomoloGene database maintained by the U.S. National Center for Biotechnology Information offered by the National Institutes of Health to determine species homologs and orthologs and their percent similarity to the newly isolated human LEC-specific genes. Analyses of the sequences were performed using a resource of curated and calculated homologs for genes as represented by UniGene or by annotation of genomic sequences, generally comparing EST and mRNA sequences from UniGene, as well as transcripts extracted from annotated genomic sequences. (Zhang, et al., J. Comp. Biol. 7:203-14. 2000). The best match for a nucleotide sequence in one organism to a nucleotide sequence in a second organism is based on the degree of similarity between the two sequences, with a minimum alignment of 100 base pairs. The similarity between the two sequences was determined by an alignment score. The alignment score for a sequence pair is the sum of the similarity scores of the sections of the two sequences that aligned.

BLAST and Pfam analysis of the isolated genes revealed numerous LEC-specific transmembrane proteins, including KIAA1972, KIAA0472, K1AA0494, ATP10D, KIAA1724, DKFZp564K142, KIAA1573, DKFZp761N1114, and FLJ25916, set out in SEQ ID NOS: 6, 8, 10, 12, 14, 16, 20, 22, 24, 42 and 43 (see Table 1).

TABLE 1
LEC-Specific Transmembrane Proteins

| | Genbank | | |
|---|---|---|---|
| | Accession | nucleotide | polypeptide |
| Protein | Number | SEQ ID NO: | SEQ ID NO: |
| KIAA1972 | XM_166279 | SEQ ID NO:5 | SEQ ID NO:6 |
| KIAA0472 | XM_290898 | SEQ ID NO:7 | SEQ ID NO:8 |
| KIAA0494 | NM_014774 | SEQ ID NO:9 | SEQ ID NO:10 |
| ATP10D | NM_020453 | SEQ ID NO:11 | SEQ ID NO:12 |
| KIAA1724 | XM_040280 | SEQ ID NO:13 | SEQ ID NO:14 |
| DKFZp564K142 | NM_032121 | SEQ ID NO:15 | SEQ ID NO:16 |
| KIAA1573 | NM_020925 | SEQ ID NO:19 | SEQ ID NO:20 |
| DKFZp761N1114 | AI332934/ | SEQ ID NO:21 | SEQ ID NO:22 |
| | NM_181644 | | |
| FLJ25916 | AK098782/ | SEQ ID NO:23 | SEQ ID NO:24 |
| | NM_207347 | | |

KIAA1972: LEC-specific gene KIAA1972 (Genbank Accession No. XM_{—}166279; SEQ ID NOS: 5 and 6), a 3540 bp mRNA, has been detected by Northern blot expression at high levels in liver, kidney, testis, spleen and ovary, at moderate levels in heart, lung, smooth muscle, pancreas, spinal cord, and fetal liver, and at low levels in brain and fetal brain. Northern blot data was derived from the electronic database of Human Unidentified Gene-Encoded (HUGE) Large Proteins maintained by Kazusa DNA Research Institute, Chiba, Japan (see Ohara et al., DNA Res. 4:53-59, 1997).

Unigene comparison of the KIAA1972 sequence indicates the protein is 36% identical [over a string of 96 amino acids (aa)] to human Ryanodine receptor 2, RYR2, which is a cardiac muscle-type ryanodine receptor calcium release channel. KIAA1972 also shows 34% identity (over a string of 110 aa) to a fragment of mouse (Mus musculus) ryanodine receptor, demonstrates 29% identity (over a sequence of 152 aa) to a Drosophila melanogaster ryanodine receptor/calcium release channel, shows 48% identity (over a string of 497 aa) to a Caenorhabditis elegans zinc finger, C3HC4 type (RING finger) protein, and shows 39% identity (over 140 aa) to an Arabidopsis thaliana hypothetical protein. KIAA1972 also demonstrates similarity to species orthologs R. norvegicus LOC291860 (92.4% similarity) and M.musculus 4930470D19Rik (87% similarity).

Protein domain mapping for the KIAA1972 sequence using the Pfam database (SwissProt designation Q96DX4) predicts a protein of 576 amino acids with a signal peptide from amino acids 1-16, a SPRY domain from approximately residues 358-482 and a RING domain from approximately residues 527-561. The SPRY domain is derived from similarities to the spla motif and the ryanodine receptor. The SPRY domains have homologues in butyrophilin/marenostrin/pyrin proteins. Ca²⁺-release from the sarcoplasmic or endoplasmic reticulum, the site of intracellular Ca²⁺ storage, is mediated by the ryanodine receptor (RyR) and/or the inositol trisphosphate receptor (IP3R).

The RING-finger domain is a specialized type of Zn-finger region of 40 to 60 amino acid residues that binds two zinc atoms, and is probably involved in mediating protein-protein interactions. RING domains contain cysteine and histidine residues for binding to the Zinc molecules in either a C3HC4-or a C₃H₂C3 configuration. E3 ubiquitin-protein ligase activity is intrinsic to the RING domain of c-Cbl and is likely to be a general function of the RING domain. E3 ubiquitin-protein ligase MdM2 expression is amplified in certain tumors (including soft tissue sarcomas, osteosarcomas and gliomas), indicating that ubiquitin-ligase function may be important in maintaining cell homeostasis and regulating cell cycle activities.

Therefore, KIAA1972 is expected to act to regulate calcium storage in lymphatic endothelial cells and to function in the regulation of cell homeostasis. Deregulation of KIAA1972 is expected to result in aberrant LEC proliferation and lymphatic disorders such as lymphedema, lymphangioma, lymphangiomyeloma, lymphangiomatosis, lymphangiectasis, lymphosarcoma, and lymphangiosclerosis.

KIAA0472: Full-length KIAA0472, first isolated from the human adult brain genome, is a 2589 bp mRNA sequence. Unigene comparison of the KIAA0472 gene (Genbank Accession No. XM_{—}290898; SEQ ID NOS: 7 and 8) indicates that the predicted protein is 65% identical (within a sequence of 102 aa) to human neuronal thread protein, shares 29% identity (over 245 aa) to M musculus death domain-containing protein RIP; and demonstrates 33% identity (over 139 aa) with rat R. norvegicus) protein kinase C, delta type, NPKC-delta. KIAA0472 also demonstrates significant identity to several other protein kinases such as D. melanogaster TGF-beta activated kinase 1, DmTak1 (27% identity over 262 aa), a C. elegans tyrosine-protein kinase (25% identity over 293 aa) and an A. thaliana protein kinase (38% identity over 161 aa).

Protein domain mapping of the predicted 390-amino-acid KIAA0472 gene (SwissProt designation 075060) reveals that a 365 amino acid fragment of the polypeptide (SEQ ID NO: 42) possesses a protein kinase domain from approximately residues 88-342. Pfam analysis predicts the domain lies at residues 88-333 while SMART analysis predicts a STYKc domain, from approximately residues 88-342. There is also a region of similarilty to a potential transmembrane domain from amino acids 262-284. The designation of the STYKc domain leads to the expectation that KIAA0472 is a protein kinase of unclassified specificity, and is more specifically expected to be a phosphotransferase exhibiting dual-specificity as a Ser/Thr/Tyr kinase. Both serine/threonine and tyrosine protein kinases share a common catalytic core comprising a glycine-rich region of amino acids in proximity to a lysine residue which has been shown to be involved in ATP binding, as well as a conserved aspartic acid residue which is important for the catalytic activity of the enzyme.

KIAA0494: KIAA0494 is a mRNA sequence of 5766 base pairs first isolated from human adult brain. Unigene comparison of the KIAA0494 sequence (Genbank Accession No. NM_{—}014774; SEQ ID NOS: 9 and 10) indicates the predicted protein is 23% identical (over a sequence of 306 amino acids) to Arabidopsis thaliana hypothetical protein F5E19_{—}70. Analysis of potential species orthologs revealed that KIAA0494 is similar to R. norvegicus LOC298425 (85.7% similarity) and M.musculus 4732418C07Rik (82.2% similarity).

Protein domain mapping using the Pfam database indicates that the 495-amino-acid KIAA0494 protein (SwissProt designation 075071) exhibits a transmembrane domain from approximately amino acids 71-93; and, coiled-coil regions from amino acids 95-115, 209-229 and 292-312. Both transmembrane and coiled coil domains are found in proteins with diverse functions. Presence of the transmembrane domains indicates the protein is membrane bound. Coiled-coil domains are structural domains composed of two or three alpha helices that wind around each other and are found in a variety of proteins.

ATP10D: Screening for differential expression of LEC- and BEC-specific genes identified the ATP10D gene (Genbank Accession No. NM_{—}020453; SEQ ID NOS: 11 and 12) as a gene upregulated in lymphatic endothelia. ATP10D was isolated as a 6550 bp mRNA sequence. Analysis of related species orthologs detects an ATP10D ortholog in R norvegicus LOC305314 (87.2%).

Unigene comparison of the ATP10D gene sequence indicates that the predicted ATP10D protein demonstrates 99% identity, over a 650 aa length of aligned region, to human phospholipid-transporting ATPase VD (ATPVD); demonstrates 56% identity, over a 580 aa alignment, to a mouse potential phospholipid-transporting ATPase VA; demonstrates 23% identity (over a 365 amino acid alignment) to rat ATPase isoform 2, Na⁺K⁺ transporting, beta polypeptide 2; shows 38% identity (604 aa alignment) to an A. thaliana putative chromatin granule ATPase II homolog; and is 41% identical (over a 570 aa alignment) to a C. elegans ATPase.

Human ATP10D is predicted to be a 1426-amino-acid protein. Protein domain mapping of the 1416 amino acid mouse homolog of human ATP10D (SwissProt designation Q8K2×1) demonstrates five transmembrane domains, from amino acids 7-25, 53-75, 88-105, 115-137, and 157-176, and also to possess a signal peptide from residues 1-23. The mouse ATP10D also contains a hydrolase domain from amino acids 805-1074. Hydrolase enzymes catalyze the hydrolysis of various molecular bonds and include such proteins as epoxide hydrolases and phosphatases.

Human ATP10D also contains predicted regions of similarity to MgtA domains involved in cation transport, from approximately residues 70-457 and residues 722-1329 of SEQ ID NO: 12, and is expected to be a cation transport ATPase, similar to other ATPase transporters that are involved in inorganic ion transport and metabolism. Other possible functions include cation transport, integral to membrane, magnesium ion binding, hydrolase activity, ATPase activity, and phospholipid-translocating ATPase activity. ATP10D is expected to play a significant role in transporting ions across the LEC membrane and regulating varied cellular activities.

KIAA1724: KIAA1724 (Genbank Accession No. XM_{—}040280; SEQ ID NOS: 13 and 14) was isolated as a LEC-specific, 8083 bp mRNA sequence. Tissue expression analysis by Northern blot (data derived from HUGE protein database maintained by Kazusa DNA Research Institute, Chiba, Japan) detected KIAA1724 transcripts at highest levels in B. amygdala, B. cerebellum, B. substantia nigra, and B. thalamus, with moderate expression in overall brain, liver, B.corpus callosum, B.caudate nucleus, B. hippocampus and the B. subthalamic nucleus. Analysis of species similarities led to the identification of species orthologs in R. norvegicus LOC298856 (88.4%) and M musculus 4933402G07Rik (84.4%)

Unigene analysis of the KIAA1724 sequence reveals that the sequence demonstrates 64% identity, over a string of 49 amino acids, to a probable human thromboxane A2 receptor isoform beta, and 34% identity, over a 377 amino acid alignment, to C. elegans hypothetical protein F54D7.2.

Protein domain mapping of a 400-residue fragment comprising the predicted 386 residue KIAA1724 protein (SwissProt designation Q9COD9) indicates the protein contains a CDP-alcohol phosphatidyltransferase (CDP-OH P transf) domain from amino acids 108-252, and 10 transmembrane domains located at residues 61-83; 98-117; 137-159; 164-186; 193-215; 235-257; 270-292; 305-324; 331-53; and 358-380. Phosphatidyltransferases are involved in phospholipid biosynthesis. Generally, these enzymes are proteins of from 200 to 400 amino acid residues exhibiting a conserved region that contains three aspartic acid residues and is located in the N-terminal region of the proteins. KIAA1724 is expected to function in phospholipid biosynthesis in LECs.

DKFZp564K142: Analysis of the genome for LEC-specific genes identified hypothetical gene DKFZp564K142 (Genbank Accession No. NM_{—}032121; SEQ ID NOS: 15 and 16), which comprises a nucleotide sequence of 2788 bp, and exhibits higher expression in LECs versus blood endothelial cells. Initial analysis of the sequence revealed DKFZp564K142 species orthologs in S. scrofa Ssc.16415 (93.9%), R. norvegicus IAG2 (87.8%) and M musculus, 2610529C04Rik (79.7%).

Unigene analysis of DKFZp564K142 indicates that the amino acid sequence of the protein is 69% identical over a 323 aa region to the protein encoded by human gene N33; is 93% identical, over 332 aa, to the rat implantation-associated protein, IAG2; and is 42% identical (over a 303 aa sequence) to the C. elegans ZK686.3 protein.

Pfam analysis of the 335 amino acid hypothetical DKFZp564K142 protein (SwissProt designation Q9HOU3) indicates that the protein comprises an OST3-OST6 domain located from approximately residues 20-330. The proteins in the yeast OST3-OST6 family are part of a complex of eight endoplasmic reticulum (ER) proteins that transfer core oligosaccharide from dolichol carrier to Asn-X-Ser/Thr motifs. This family includes both OST3 and OST6, each of which contains four predicted transmembrane helices. Disruption of OST3 and OST6 leads to a defect in the assembly of the protein complex. Hence, the function of these genes is expected to be essential for recruiting a fully active complex necessary for efficient N-glycosylation.

The DKFZp564K142 protein also contains a signal peptide at amino acids 1-29 and multiple transmembrane domains, similar to either the OST3 or OST6 protein, which are located at approximately amino acids 7-29, 186-205,212-234, 266-288, and 301-320. Both rat IAG2 and DKFZp564K142 exhibit five transmembrane domains. Based on the high similarity to rat IAG2, DKFZp564K142 is expected to be a human homolog to implantation-associated protein involved in pregnancy and may play a role in establishing lymphatic endothelial cells during pregnancy and embryo implantation.

KIAA1573: KIAA1573 (Genbank Accession No. NM_{—}020925; SEQ ID NOS: 19 and 20) encodes a LEC-specific, hypothetical human protein and is approximately 5565 base pairs in length. Northern blot analysis of mRNA expression (data derived from HUGE protein database maintained by Kazusa DNA Research Institute, Chiba, Japan) shows that KIAA1573 is expressed at high levels in B. thalamus, with lower expression in other tissues, such as B. caudate nucleus, B. hippocampus, B. subthalamic nucleus, B.substantia nigra, ovary, brain and kidney. Comparison of KIAA1573 to other species leads to the expectation of orthologs such as R. norvegicus LOC298267 (90.6%), B. taurus Bt.1428 (88.6%) and M. musculus 1190007F10Rik (83.2%).

Unigene analysis of the predicted amino acid sequence of KIAA1573 reveals that the protein demonstrates 22% identity (over a 598 aa sequence) to human calcium channel, voltage-dependent, alpha 2 delta calcium channel subunit; demonstrates 22% identity (within a sequence of 676 aa) to mouse calcium channel, voltage-dependent, alpha 2 delta calcium channel subunit; and 19% identity (over 677 aa) to rat CIC2_RAT Dihydropyridine-sensitive L-type, calcium channel alpha-2/delta subunit precursor.

Pfam protein domain analysis of a 1185 residue polypeptide sequence (SEQ ID NO: 43) comprising the predicted 985 amino acid KIAA1573 protein (Swissprot designation Q9HCJ9) reveals two Cache domains in KIAA1573, a full Cache domain from amino acids 364-443 and a partial domain from 683-764. The protein also possesses a single transmembrane domain spanning residues 1006-1028 of SEQ ID NO: 43, and is expected to be expressed on the cell surface. The cache motif is a signaling domain that is found in animal calcium channel subunits and a certain class of prokaryotic chemotaxis receptors.

Analysis of the protein sequence using the Smart [Simple Modular Architecture Research Tool] database predicts the presence of a VWA domain from residues 137-333. The VWA domain is derived from von Willebrand factor A, which is a large multimeric glycoprotein found in blood plasma. This vWF domain is found in various plasma proteins: complement factors B, C2, CR3 and CR4; the integrins (1-domains); collagen types VI, VII, XII and XIV; and other extracellular proteins. The majority of VWA-containing proteins are extracellular. A common feature of proteins with this domain appears to be involvement in multiprotein complexes. Proteins that incorporate vWF domains participate in numerous biological events (e.g. cell adhesion, migration, homing, pattern formation, and signal transduction), involving interaction with a large array of ligands. VWA domains in extracellular eukaryotic proteins mediate adhesion via metal ion-dependent adhesion sites (MIDAS). A number of human diseases arise from mutations in VWA domains, including diseases of the lymphatic endothelium, such as bleeding disorders. The similarity of KIAA1573 to extracellular, adhesion type proteins leads to the expectation that it acts as a mediator of cell signaling, while the presence of the VWA domain implicates KIAA1573 as a useful target in regulating LEC function in lymphatic endothelium mediated bleeding disorders.

DKFZp761N1114: DKFZp761N1114 (Genbank Accession No. AI332934, NM_{—}181644; SEQ ID NOS: 21 and 22) has a nucleotide sequence of 3352 bp. Tissue expression analysis of human tissues, as detected by Multiple Tissue Northern Blot (Clontech) of adult human RNA, indicates that DKFZp761N1114 produces a 4.5 kb transcript, which is expressed at high levels in the stomach, kidney, and brain, with lower expression in other tissues, such as skeletal muscle, colon, placenta, lung, tongue and trachea. Comparison of the sequence with other species leads to expected orthologs in R norvegicus LOC304787 (85.9%) and M musculus A930031D07Rik (80.3°/O).

Unigene analysis of the predicted protein sequence shows that DKFZp761N1114 is 60% identical (over a 100 aa sequence) to human neuronal thread protein.

Protein domain mapping reveals the 403 amino acid DKFZp761N1114 (SwisProt designation Q8N468) protein contains a signal sequence and 7 apparent transmembrane domains, from amino acids 27-49; 108-130; 194-216; 236-258; 265-287; 291-313; 326-348; and 353-375. DKFZp761N1114 may also contain a fucose permease domain from approximately residues 202-376, which is involved in carbohydrate transport and metabolism. As such, DKFZp761N1114 is expected to act as a unique seven-transmembrane receptor on the surface of LECs and to modulate or regulate carbohydrate activity in the cell.

Further analysis revealed that the DKFZp761N1114 gene encodes an alternative predicted protein structure which includes a transmembrane protein with only a single transmembrane domain and a MSF-1 domain (Major Facilitator Superfamily), a domain present for example in glycerol-3-phosphate transporter from E. coli bacteria. An additional alternative predicted domain structure indicates DKFZp761N1114 may encode twelve transmembrane domains and an amino-terminal signal peptide.

The gene is expressed in lymphatic endothelial cells (LECs), but not in blood vascular endothelial cells (BECs), in an in vitro cell culture of primary endothelial cells (HDMEC, PromoCell).

In vivo, the gene is expressed, as revealed by non-radioactive in situ hybridization (NR-ISH), in lymphatic vessels of mouse embryonic mesenterium at E17.5, but is not detectable in blood vessels. The antisense transcript, as detected by NR-ISH using the sense probe, is also expressed in the mesenteric lymphatic vessels, but to a lesser extent. Thus, the expression of DKFZp761N1114 is expected to be modulated at least in part, by its antisense transcript. Using frozen sections from E16.5 mouse embryos, very little expression in embryonic mouse tissues was detected.

A Multiple Tissue Northern Blot (Clontech) of adult mouse RNA, indicates the DKFZp761N1114 gene is expressed in kidney and to a lesser extent in liver, brain and heart, but not in lung, testis, spleen or skeletal muscle. NR-ISH analysis showed that the gene was expressed in collecting tubules in the kidney of adult mice.

The expression of DKFZp761N1114 in kidney tubules and its domain structure, involving a Major Facilitator Superfamily (MFS) domain, indicate a function in uptake of substances from the extracellular space in kidney, as well as in lymphatic vessels.

FLJ25916: Screening of the LEC library also identified hypothetical protein FLJ25916 (Genbank Accession No. AK098782; SEQ ID NOS: 23 and 24) as a LEC-specific gene. FLJ25916 was isolated from fis, clone CBR04903 as a 1893 bp fragment.

Comparison of the putative coding region of the FLJ25916 mRNA to other sequences reveals that FLJ25916 is similar to human hypothetical protein KIAA0233 (44% identity over a 572 amino acid sequence) and is 28% identical (over 315 aa) to C elegans protein T20D3.11.p. KIAA0233, to which FLJ25916 shows significant identity, demonstrates homology to both the mouse collagen alpha chain precursor and rat procollagen (26% identity with both proteins, over a sequence of approximately 1005 aa). Ortholog analysis for FLJ 25916 predicts similarity to mouse protein ENSMUSP00000059624 (novel Ensembl prediction).

Protein domain mapping of the hypothetical 583 amino acid FLJ25916 protein (SwissProt designation Q8N787) identified a signal peptide from residues 1-18, nine transmembrane domains located at approximately amino acids 2-19; 50-72; 220-242; 247-264; 269-291; 340-362; 401-423; 427-444; and 457-479, and also predicted a coiled-coil region from amino acids 169-189. The similarity of FLJ25916 to other mammalian collagen-like proteins leads to the expectation that it acts as a structural protein and/or interacts with the extracellular matrix surrounding the LEC.

Further analysis shows that FLJ25916, also identified as C18orf30, is expressed in lymphatic endothelial cells (LECs) and to a lesser extent in blood vascular endothelial cells (13ECs) in an in vitro cell culture of primary endothelial cells (HDMEC, PromoCell). In vivo, the gene is expressed in the trigeminal ganglion in the brain, in the eye and in bone and is expressed weakly in submandibular gland, kidney and lung, as revealed by non-radioactive in situ hybridization (NR-ISH) of frozen sections from E16.5 mouse embryos.

The above transmembrane and cell-surface-expressed proteins are useful for the identification and isolation of LEC, and useful for regulation of LEC activity.

### Example 2

### IDENTIFICATION OF INTRACELLULAR PROTEINS EXPRESSED DIFFERENTIALLY IN LEC

Assays using the microarray and LEC cDNA libraries identified several LEC-specific genes encoding proteins expressed on the cell surface and which are suitable targets for identification and localization of lymphatic endothelial cells, as well as being suitable targets for the manipulation of their function and activity. In addition to these cell-surface LEC proteins, intracellular proteins were identified as being LEC-specific; these proteins are expected to contribute to the regulation of the intracellular function of LEC genes, such as the transcription of particular genes and the transport of necessary proteins through the intracellular space.

By way of example, the analysis of LEC-specific intracellular proteins identified the following genes as being expressed at higher levels in LECs versus BECs: HERC3, KIAA1340, protein FLJ20051, protein FLJ13910, protein FLJ1029, KIAA1102, protein FLJ32029, protein FLJ23594, and protein FLJ10532 (see Table 2).

TABLE 2

LEC-Specific Transcription Factors or
Intracellular Proteins

| | Genbank | | |
|---|---|---|---|
| | Accession | nucleotide | polypeptide |
| Protein | Number | SEQ ID NO: | SEQ ID NO: |
| HERC3 | NM_014606 | SEQ ID NO:27 | SEQ ID NO:28 |
| KIAA1340 | XM_044836 | SEQ ID NO:29 | SEQ ID NO:30 |
| FLJ20051 | NM_019087 | SEQ ID NO:31 | SEQ ID NO:32 |
| FLJ13910 | NM_022780 | SEQ ID NO:17 | SEQ ID NO:18 |
| FLJ11029 | NM_018304 | SEQ ID NO:33 | SEQ ID NO:34 |
| KIAA1102 | XM_044461 | SEQ ID NO:35 | SEQ ID NO:36 |
| FLJ32029 | NM_173582 | SEQ ID NO:37 | SEQ ID NO:38 |
| FLJ23594 | NM_024781 | SEQ ID NO:39 | SEQ ID NO:40 |
| FLJ10532 | AK001394 | SEQ ID NO:41 | |
| BIKE protein | NM_017593 | SEQ ID NO:25 | SEQ ID NO:26 |

HERC3: Screening for LEC-specific genes identified the HERC3 gene as a gene upregulated preferentially in LECs versus BECs. Unigene comparison of the HERC3 sequence (Genbank Accession No. NM_{—}014606; SEQ ID NOS: 27 and 28) indicates that the predicted protein is 95% identical (over 891 aa) to human hypothetical protein 2 (fragment); 35% identical (over 337 aa) to mouse rjs protein; shares 31% identity (over 184 aa) to rat ubiquitin ligase Nedd4; demonstrates 37% identity (over 309 aa) to the A. thaliana UVB-resistance protein, UVR8; is 24% identical (over a 330 aa sequence) to D. melanogaster chromatin-binding protein BJ1; and is 30% identical (over 891 aa) to C elegans hypothetical protein Y48G8AL.1.p.

HERC3 protein domain mapping (SwissProt designation Q15034) reveals four regulator-of-chromosome-condensation (RCC1)-like protein domains found from residues 52-99, 102-152, 155-205, and 208-257. Regulator-of-chromosome-condensation (RCC1) is a eukaryotic protein that binds to chromatin and interacts with ran, a nuclear GTP-binding protein, to promote the loss of bound GDP and the uptake of fresh GTP, thus acting as a guanine-nucleotide dissociation stimulator (GDS). The interaction of RCC1 with ran is believed to play an important role in the regulation of gene expression. RCC1 is a protein that contains seven tandem repeats of a domain of about 50 to 60 amino acids. The RCC1-type of repeat is also found in the X-linked retinitis pigmentosa GTPase regulator.

Mapping of the HERC3 protein also reveals a HECT domain spanning approximately amino acids 721-1050, Pfam predicting the domain from amino acids 752-1050 and SMART analysis detecting the domain at residues 721-1050. HECT (Homologous to the E6-AP Carboxyl Terminus) domains are typically involved in ubiquitin ligase activity. Proteins containing this domain at the C-terminus include ubiquitin-protein ligase, which regulates ubiquitination of CDC25, and directly transfers ubiquitin to targeted substrates. Human thyroid receptor interacting protein 12 also contains this domain and is a component of an ATP-dependent multi-subunit protein that interacts with the ligand-binding domain of the thyroid hormone receptor. This protein may have similar function to an E3 ubiquitin-protein ligase such as human ubiquitin-protein ligase E3A, which interacts with the E6 protein of the cancer-associated human papillomavirus types 16 and 18. A cysteine residue in the domain is required for ubiquitin-thiolester formation. The HECT domain is known to form complexes with the ubiquitin-conjugating domain of other proteins, and this domain is involved in ubiquinating proteins to signal their degradation by the proteosome.

Further analysis of protein domains in HERC3 indicate that a region from residues 333-481 is typically associated with proteins containing the classical zinc finger C₂H₂ domain; and residues 482-605 represent a domain associated with proteins containing lectin c domains.

Based on the foregoing information, HERC3 is expected to function as a regulator of protein degradation in LECs, given the similarity to ubiquitin-ligase proteins. HERC3 is also expected to be involved in chromatin binding, based on the presence of the RCC1 domain.

KIAA1340: Northern blot expression of KIAA1340 (Genbank Accession No. XM_{—}044836; SEQ ID NOS: 29 and 30) indicates that KIAA1340 mRNA (approximately 4363 bp) is expressed at the highest levels in brain and at moderate levels in lung, ovary and spinal cord. In situ expression measured in day 16.5 mouse embryo indicates that the transcript is expressed at the highest level in the thymus and the intestine of developing mice. Expected orthologs of KIAA1340 include R norvegicus, LOC312860 (86.9%), and M. musculus, C230080120R1k (82.4%)

Unigene comparison of the KIAA1340 gene sequence indicates that the protein product of KIAA1340 demonstrates 33% identity, over a 155-aa length of aligned region, to mouse Kelch-like ECH-associated protein 1 (Cytosolic inhibitor of Nrf2); shares 32% identity, over a 155-amino-acid alignment, with rat Kelch-like ECH-associated protein 1 (Cytosolic inhibitor of Nrf2, INrf2); has 36% identity, over 98 aa, with the A. thaliana hypothetical protein F7A7.180; has 25% identity, over a 278-aa alignment, with D. melanogaster long-form kelch protein; and is 27% identical (over a 151-aa alignment) to C elegans hypothetical protein W02G9.2.

Pfam analysis using a 410 amino acid fragment (SEQ ID NO: 44) of the predicted 505 amino acid KIAA1340 protein (SwissProt designation Q8N334) indicates KIAA1340 exhibits similarity to Kelch-related proteins and contains Kelch domains from amino acids 147-193 and 195-236, and partial Kelch domains from residues 238-277 and 291-340. Comparison domain mapping using the SMART database predicts Kelch domains from 159-206, 207-249, and 250-296. Identification of Kelch domains using SMART analysis reveals the Kelch domains to be located from residues 159-206, 207-249 and 250-296. The Kelch motif is an approximately 50-residue domain, named after the Drosophila mutant in which it was first identified, and is found six times in Drosophila egg-chamber regulatory protein, in mouse protein MIPP, in a number of poxviruses, in α- and β-scruin, and in galactose oxidase from the fungus Dactylium dendroides. The known functions of kelch-containing proteins include: scruin functioning as an actin cross-linking protein; galactose oxidase catalyzing the oxidation of the hydroxyl group at the C6 position in D-galactose; neuraminidase hydrolyzing sialic acid residues from glycoproteins; and kelch from Drosophila having a cytoskeletal function in the developing oocyte.

KIAA1340 also demonstrates a BTB/POZ domain at approximately amino acids 6-66 of SEQ ID NO: 44. The BTB (for BR-C, ttk and bab) or POZ (for Pox virus and Zinc finger) domain is present near the N-terminus of a fraction of zinc-finger proteins and in proteins that contain the motif, such as Kelch and a family of pox virus proteins. The BTB/POZ domain mediates homomeric dimerization and, in some instances, heteromeric dimerization. These domains are known to be protein-protein interaction domains found at the N-termini of several C₂H₂-type transcription factors, as well as Shaw-type potassium channels. POZ domains from several zinc-finger proteins have been shown to mediate transcriptional repression and to interact with components of histone deacetylase co-repressor complexes, including N-CoR and SMRT.

The presence of the Kelch domains in KIAA1340 indicates the protein may have several different functions, while the presence of the Zinc-binding domain implicates KIAA1340 in protein-protein interactions, which are expected to be involved in the regulation of DNA binding and transcription.

FLJ20051: FLJ20051 exhibits a coding region of 2501 bp (Genbank Accession No. NM_{—}019087; SEQ ID NOS: 31 and 32) and is expressed as a 4.3 kb transcript. Multiple Tissue Northern Blot (Clontech) of adult human RNA demonstrates that the FLJ20051 transcript is expressed at highest levels in kidney, placenta, heart, and skeletal muscle, and lower levels in the brain, lung, liver, colon, small intestine, thymus, peripheral blood leukocytes, and spleen.

Whole mount in situ analysis, as well as non-radioactive in situ hybridization (NR-ISH), of mouse mesenterium (E17.5, E18.5 or P6) detected FLJ20051 expression in lymphatic vessels of the gut mesenterium but not in arteries or veins, i.e., blood vessels. The antisense transcript, as detected by NR-ISH using the sense probe, is also expressed in the mesenteric lymphatic vessels, but to a lesser extent. These data indicate that the expression of FLJ20051 is controlled, at least in part, by its antisense transcript. These data also indicate that FLJ20051 plays a role in the development of the lymphatic endothelium.

Using in situ hybridization of frozen sections from E16.5 mouse embryos, FLJ20051 expression was detected in lung, kidney, brain and hair follicles in the skin. Multiple Tissue Northern Blot (Clontech) of adult mouse RNA revealed that the gene is expressed in kidney, and to a lesser extent in heart, brain and liver, and in still lesser extent in lung and testis, with no detectable expression in spleen or skeletal muscle.

Unigene comparison of the FLJ20051 gene sequence indicates that the predicted protein demonstrates 35% identity (over a 159-amino-acid alignment region) to a mouse homolog of human ADP-ribosylation factor 6; demonstrates 35% identity (over 159 aa) to the rat homolog of ADP-ribosylation factor 6; is 33% identical (over a 160-aa sequence alignment) to A. thaliana probable ADP-ribosylation factor At2g18390; shows 35% identity (over a 154-aa alignment) to ADP-ribosylation factor 3 of D. melanogaster; and is 34% identical (over a 168-aa sequence) to C elegans ADP-ribosylation factors of the Arf family, which contains an ATP/GTP-binding P-loop. FLJ20051 orthologs are R. norvegicus LOC310106, (86.4% similarity), and M musculus C230032K13R1k, (85.1% similarity).

Homology/Unigene data indicates that FLJ20051 acts as an ADP ribosylase. Domain mapping revealed that the 204-amino-acid protein (SwissProt designation Q9NXUS) contains GTP binding site from approximately residues 8-198. Pfam predicts an ADP-ribosylation factor (arf) domain from approximately residues 17 to 194 while SMART analysis predicts the GTPase region lies form amino acids 8-198. Small ADP ribosylation factor GTP-binding proteins are key regulators of vesicle biogenesis in intracellular trafficking. Members of the Arf family include Arl (Arf-like), Arp(Arf-related proteins) and the related Sar (Secretion-associated and Ras-related) proteins. Arf proteins cycle between inactive GDP-bound and active GTP-bound forms that bind selectively to effectors. The Arf domains can form complexes with GMP-PDE delta domains, which are believed to be specific soluble transport factors for certain prenylated proteins; Arl2-GTP is a regulator of PDE-mediated transport. Arf6 is found to be involved in cell motility, membrane dynamics, actin reorganization and tumor cell invasion.

Based on the foregoing information, FLJ20051 is expected to be an ADP Ribosylase active in lymphatic endothelial cells and to regulate GDP/GTP binding and intracellular trafficking, membrane dynamics, cell motility, or actin reorganization. FLJ20051 has recently been named ARFRP2: ADP-ribosylation factor related protein 2 (NM_{—}019087).

FLJ13910: Microarray analysis of LEC-specific genes identified hypothetical protein FLJ13910 as having greater expression levels in LEC versus BEC. This gene was also detected in a putative LEC library. Further, the gene is expressed in lymphatic endothelial cells (LECs), but not in blood vascular endothelial cells (BECs) in an in vitro cell culture of primary endothelial cells (HDMEC, PromoCell), confirming that FLJ13910 is a LEC-specific gene.

Protein FLJ13910 (Genbank Accession No. NM_{—}022780; SEQ ID NOS: 17 and 18) is encoded by an mRNA sequence of 3239 base pairs. Unigene analysis of the amino acid sequence of this mRNA indicates that FLJ13910 shows identity to the mouse erythroblast macrophage protein (26% identity over a 261-aa sequence), shows 37% identity (over 320 aa) to a putative A. thaliana WD repeat protein; and demonstrates 25% identity (within a 340-aa sequence) to C. elegans T07D1.2.p. Species orthologs of FLJ13910 are found in R. norvegicus LOC312439 (94.5%), and in M musculus 1110007A06Rik (83.8%).

Protein domain analysis of the 391-residue FLJ13910 protein (SwissProt designation Q9H871) revealed a LisH domain from amino acids 114-146 and a CTLH domain from approximately amino acids 153-210. The lissencephaly type-1-like homology motif (LisH) is an alpha-helical motif present in Lis1, treacle, Nopp140, some katanin p60 subunits, muskelin, tonneau, LEUNIG and WD40 repeat-containing proteins. It is believed that LisH motifs contribute to the regulation of microtubule dynamics, either by mediating dimerization, or by binding either a cytoplasmic dynein heavy chain or microtubules. The CTLH domain forms an alpha-helix. This domain is found in the mouse and human erythroblast macrophage proteins, both mouse and rat muskelin proteins, in the A thaliana putative WD repeat protein. Muskelin is a mediator of cell-adhesive behavior and cytoskeletal organization (Adams et al., EMBO J. 17:4964-74, 1998), whereas WD-repeat proteins are a group of structurally related proteins that participate in a wide range of cellular functions, including transmembrane signaling, mRNA modification, vesicle formation, and vesicular trafficking. Protein domain mapping predicts an E3 ubiquitin ligase domain (KOG2817)), which is indicative of post-translational modification, protein turnover or chaperone function.

Based on the protein domain mapping analysis, which indicates structural similarity to several actin binding or cytoskeleton related proteins, FLJ13910 is expected to act as a modulator of LEC migration and localization. FLJ13910 is also expected to act as an internal regulator of vesicle formation and vesicle trafficking, based on structural similarity to WD domain-containing proteins. Given the similarity to the muskelin protein, FLJ13910 is also expected to participate in structural and adhesion activities in LECs and plays a role in development and migration of LECs.

In vivo, NR-ISH of frozen sections from E16.5 mouse embryos detected gene transcript in the trigeminal ganglion, brain, nasal cavities, intestine, lung, submandibular gland and kidney.

FLJ11029: Protein FLJ11029 (Genbank Accession No. NM_{—}018304; SEQ ID NOS: 33 and 34) is encoded by a 2334 bp mRNA sequence. Unigene analysis of the amino acid sequence indicates that FLJ11029, which is upregulated in LEC compared to BEC, is a protein that shows no sequence similarity to any gene disclosed in a public database. Database exploration for species orthologs indicates that FLJ11029 is similar to R norvegicus—LOC287597 (81.8%), and M musculus B930067F20Rik (81.6%).

Protein domain mapping of the predicted 360 amino acid FLJ11029 (SwissProt designation Q9NUZ7) indicates that the amino acid sequence contains a domain at approximately residues 1-150 that is similar to domains associated with the PmbA_TldD family of proteins. It has been suggested that the TldD and PmbA proteins modulate the activity of DNA gyrase, or that PmbA may be involved in secretion. Pfam mapping also indicates FLJ11029 amino acids 151-360 are associated with domains found in multiple species of bacteria, the Bac surface Antigen, and are related to the UPF0140 family of eukaryotic proteins. Additional analysis indicates that this protein contains one of the following domains: Zinc finger, C3HC4 type (RING finger), ZPR1 zinc-finger domain, or C5HC2 zinc finger, which is expected to confer a DNA-binding function.

Protein domain mapping leads to the expectation that protein FLJ11029 acts as a DNA-binding/DNA-regulating protein involved in LEC transcriptional regulation.

KIAA1102: KIAA1102 (XM_{—}44461; SEQ ID NO: 35 and 36) is encoded by a 6743 bp mRNA sequence. The gene is expressed in lymphatic endothelial cells (LECs), but not in blood vascular endothelial cells (BECs) in an in vitro cell culture of primary endothelial cells (HDMEC, PromoCell).

KIAA1102 is expressed as a 6.5 kb transcript in most tissues and as a 8.5 kb transcript in skeletal muscle. Expression in human tissue, as assayed by Northern blot analysis using Multiple Tissue Northern Blot (Clontech), is highest in heart, brain, skeletal muscle, and lung, with intermediate levels of expression found in prostate, thyroid, spleen, and placenta, and lower levels in kidney, small intestine, tongue and trachea. Assessment of KIAA1102 homologs in other species identified orthologs in B. taurus Bt.10388 (87.1%), R norvegicus LOC305332 (84.0%), and M musculus LOC269654 (81.6%).

Analysis of protein similarity using the Unigene database reveals that KIAA1102 is 28% identical (over 308 amino acids) to human LIM domain only 7 isoform; is 23% identical (over a sequence of 197 aa) to mouse sciellin; is 24% identical (over 293 aa) to C elegans F28F5.3b.p; and is 19% identical (over a sequence of 1371 aa) to the D. melanogaster T13564 microtubule-associated protein homolog.

Protein domain mapping of a 1101 fragment (SEQ ID NO. 45) of the predicted 1427 residue KIAA1102 protein (SwissProt designation Q9UPQ0) reveals similarity to a Calponin-like actin-binding domain (CH domain) from approximately residues 41-156; more particularly, Pfam analysis predicts the domain lies at residues 41-156 and SMART predicts a CH domain from amino acids 41-142. Mapping also detected a Zn-binding protein domain (LIM) spanning approximately amino acids 1030-1094 of SEQ ID NO: 45, and more specifically with Pfam analysis from aa 1031-1094, or with SMART analysis from residues 1030-1088.

The LIM domain is a conserved cysteine-rich domain of about 60 amino acids. This type of zinc-binding domain is present in diverse proteins, such as the homeobox protein Lim-1, the insulin gene enhancer binding protein Isl-1, and mammalian LH-2, a transcriptional regulatory protein involved in the control of cell differentiation in developing lymphoid and neural cell types. Some LIM domains bind protein partners via tyrosine-containing motifs. LIM domains are found in many key regulators of developmental pathways.

The calponin homology domain defines a superfamily of actin-binding domains found in both cytoskeletal proteins and signal transduction proteins, and is similar to a region in many actin-binding proteins, including spectrin, alpha-actinim and fimbrin, which contain a 250-amino-acid stretch called the actin binding domain(ABD). The ABD is believed to have arisen from duplication of a domain that is also found in as a single copy in a number of other proteins, like calponin or the vav proto-oncogene, and has been called the calponin homology (CH) domain. CH-domain-containing proteins are divided into monomeric actin cross-linking molecules, dimeric cross-linking proteins (e.g., alpha-actinin, beta-spectrin, and filamin) and monomeric F-actin binding proteins (dystrophin, utrophin). Each single ABD, comprising two CH domains, is able to bind one actin monomer. The CH domain also occurs in a number of proteins not known to bind actin, a notable example being the vav-protooncogene. Examples of proteins containing CH domains include alpha-actinins, calponins, human dystrophin, mammalian vav proto-oncogene, human ras GTPase activating-like protein IQGAP1, Spectrin beta chain, Transgelins, and T-plastin.

Given the protein domain homology data above, it is expected that KIAA1102 functions as a DNA control protein using its Zinc binding domain and this protein may regulate development of LECs. Additionally, the KIAA1102 protein comprises a domain similar to those in actin-binding proteins and is expected to act as a regulator of cell motility.

In vivo, non-radioactive in situ hybridization (NR-ISH) of frozen sections from E16.5 mouse embryos showed KIAA1102 gene expression in lung, kidney glomerulus, thymus, brain, lips and bone marrow.FLJ32029: The protein-coding region of protein FLJ32029 (phosphoglucomutase 2-like 1 (PGM2L1), NM_{—}173582; SEQ ID NOS: 37 and 38) lies within a 8456 bp mRNA sequence. The FLJ32029 mRNA is expressed as a 9.5 kb mRNA that is detected in brain and skeletal muscle. Comparison of the sequence to other mammalian species identified orthologs in R. norvegicus LOC293145 (84.9% similar) and M musculus 4931406N15R1k (83.9%).

Unigene analysis revealed that FLJ32029 is 59% identical (over 596 aa) to human protein FLJ10983; 44% identical (over 600 aa) to a C. elegans phosphoglucomutase and phosphomannomutase phosphoserine; and 39% identical (over 576 aa) to S. cerevisiae protein YMR278w.

Mapping for potential protein family domain similarities indicates the predicted 622-amino-acid FLJ32029 protein (SwissProt designation Q96MQ7) comprises multiple phosphoglucomutase/phosphomannomutase domains (PGM-PMM-I and PGM-PMM II) at approximately amino acid residues 61-217 (alpha/beta/alpha domain I); amino acids 237-349 (alpha/beta/alpha domain II); amino acids 428-519 (alpha/beta/alpha domain III); and amino acids 520-617, similar to the phosphomannomutase C-terminal domain, which is involved in intramolecular transferase activity (e.g., phosphotransferase activity) and carbohydrate transport and metabolism. FLJ32029 is expected to function in carbohydrate regulation in lymphatic endothelial cells and a dysfunctional FLJ32029 gene is expected to be involved in the development of lymphedema as a result of improper carbohydrate metabolism (Drubaix et al., Int J Exp Pathol. 78:117-21. 1997).

The identity of FLJ32029 as an LEC-specific gene was confirmed in an in vitro culture, where the gene was detected in lymphatic endothelial cells (LECs), and to a lesser extent in blood vascular endothelial cells (BECs) in cell culture of primary endothelial cells (HDMEC, PromoCell).

In vivo analysis of frozen sections from E16.5 mouse embryos (NR-ISH), detected gene expression in brain, bone marrow, glomeruli of the kidney cortex, thymus and intestine.FLJ23594: The protein-coding region of FLJ23594 (C18orf14, chromosome 18 open reading frame 14, NM_{—}024781; SEQ ID NOS: 39 and 40) lies within a 2015 bp mRNA sequence. The 3 kb FLJ23594 transcript has been found to be expressed in human tissue at high levels in the placenta, lung, lymph node, and adrenal gland, as detected using a Multiple Tissue Northern Blot (Clontech) of adult human RNA. In vitro analysis shows that FLJ23594 was detected in lymphatic endothelial cells (LECs) and, to a lesser extent, in blood vascular endothelial cells (BECs) in cell culture of primary endothelial cells (HDMEC, PromoCell).

Comparison of the amino acid sequence with known amino acid sequences reveals that FLJ23594 shows 23% identity (over 271 aa) to human myosin heavy chain, nonmuscle type B (Cellular myosin heavy chain, type B); demonstrates 22% identity (over 271 aa) to mouse myosin heavy chain, nonmuscle type B; is 22% identical (over a 271-aa sequence) to rat myosin heavy chain, nonmuscle type B; shows 24% identity (over 300 aa) to D. melanogaster myosin heavy chain 2; shows 23% identity (over 266 aa) to S. cerevisiae myosin-1 isoform (Type II myosin); shows 22% identity (over 269 aa) to an A. thaliana probable centromere protein; and is 22% identical (over a sequence of 284 aa) to C. elegans protein F52B10.1.

Protein domain mapping of FLJ23594 (SwissProt designation Q9H₅C1) reveals a protein of 297 amino acids having two regions of similarity to bacterial M protein, from amino acids 148-168 and 216-236. Mapping also indicates regions of similarity, at aa 153-296, to the bZIP leucine zipper. The bZIP superfamily of eukaryotic DNA-binding transcription factors groups together proteins that contain a basic region mediating sequence-specific DNA-binding followed by a leucine zipper required for dimerization. bZIP domains usually bind a pallindromic six-nucleotide site, but the specificity can be altered by interaction with an accessory factor. Some proteins that contain the bZIP domain include AP-1, oncogene v-jun, Jun-B and jun-D, probable transcription factors which are highly similar to jun/AP-1, and mammalian cAMP response element (CRE) binding proteins CREB, CREM, ATF-1, ATF-3, ATF-4, ATF-5, ATF-6 and LRF-1.

The similarity to bZIP-containing proteins indicates FLJ23594 acts as a DNA-binding protein and functions in the regulation of DNA transcription. The ability to regulate LEC-specific DNA-binding proteins or transcription factors allows for manipulation of LEC development, wherein the LEC can be induced to grow and proliferate by increased transcription or where the LEC can be induced to halt growth by blocking DNA regulatory functions. The structural similarity to cellular myosin also implicates FLJ23594 in cytokinesis, cell shaping, and specialized functions such as secretion and capping.

FLJ10532: Protein FLJ10532 (Genbank Accession No. AK001394; SEQ ID NO: 41) is encoded by a nucleic acid that was isolated as a 2372 bp mRNA fragment from fis, clone NT2RP2001044. The 2.3 kb transcript may be part of the message encoding protein DKFZp762K222. Cross-species comparison identified possible orthologs for DKFZp762K222 as R. norvegicus LOC306459 (88.3%), and M musculus 4933409N07Rik (87.7%). Cell culture of primary endothelial cells (HDMEC, PromoCell) indicates the FLJ10532 transcript is expressed in lymphatic endothelial cells (LECs) and is undetectable blood vascular endothelial cells (BECs).

BIKE: Identified herein as a LEC-specific gene, the human BIKE gene (Genbank Accession No. NM_{—}017593; SEQ ID NOS: 25 and 26), a homolog of mouse BMP-2 kinase, exhibits a 2763 bp nucleotide sequence expressed as a 7.5 kb transcript. Northern blot analysis indicates that BIKE is expressed at high levels in skeletal muscle, tongue, lymph node, colon, and placenta, and at lower levels in heart, thyroid and stomach. BIKE demonstrates 93% identity, over a region of 329 aligned amino acids, to mouse Bmp2-inducible kinase; 39% identity, over a 289-residue alignment, to rat cyclin G-associated kinase; 40% identity, over a 270-aa alignment, to Arabidopsis thaliana hypothetical protein T21L14.21; 65% identity, over a 289-aa region, to D. melanogaster Nak-P1; and 41% identity, over a 291-residue alignment, to a C. elegans serine/threonine kinase.

Protein domain mapping by Pfam analysis of a 661′ residue human polypeptide sequence (SEQ ID NO: 46) comprising the predicted 355 amino acid BIKE protein [SwissProt designation Q9NSY1 (human), Q91Z96 (Mouse, 1138 residue fragment)] indicates the protein contains a protein kinase domain located from approximately residues 51 to 314. SMART domain predicts this domain lies from approximately residues 51-317. Thus, the protein kinase domain lies at approximately amino acids 51-317. SMART analysis indicates the domain is a STYKc domain, as in KIAA0472, which are expected to be phosphotransferases that exhibit dual-specificity as Ser/Thr/Tyr kinases.

The BIKE gene product is the human homolog of mouse BMP-2-inducible kinase. Bone morphogenic proteins (BMPs) play a key role in skeletal development and patterning. Expression of the mouse gene is increased during BMP-2-induced differentiation and the gene product is a putative serine/threonine protein kinase containing a nuclear localization signal. Therefore, the protein encoded by the human homolog is expected to be a protein kinase with a putative regulatory role in osteoblast differentiation. The unexpected identification of the BIKE protein as a LEC-specific gene reveals that the BMP homolog is involved in development of the lymphatic system, as well as osteogenesis.

The manipulation of lymphatic endothelial-cell-specific molecules is expected to be applicable to treatments of LEC diseases disorders associated with tissue edemas. Without wishing to be bound by theory, manipulation of such molecules is expected to modulate endothelial cell-cell or cell-matrix protein interactions and/or to affect transendothelial transport, thereby altering the state of fluid transport across the lymphatic vessel wall. Further, such molecules provide targets for the delivery of therapeutic compounds, such as growth factors, mitogens, and the like, as well as cytostatic or cytotoxic agents known in the art. These therapeutic compounds are targeted to such cells by associating a therapeutic agent with, e.g., a binding partner (such as an antibody) of the LEC surface marker. The transmembrane proteins identified herein also provide useful targets for modulating cell adhesion or extracellular events integral to lymphatic endothelial cell function.

### Example 3

### EX-VIVO CELL STIMULATION AND GENE THERAPY FOR LYMPHEDEMA WITH LEC-SPECIFIC GENE-TRANSFECTED CELLS

The ability of LEC-specific transcription factors to regulate genes specifically involved in LEC development provides a basis for treatment of individuals exhibiting a LEC disorder or condition resulting from either an increase or decrease in LEC gene expression levels. For example, mouse pups with a homozygous mutation in transcription factor Sox18 that disrupts formation of the Sox-18/MEF2C complex (Hosking, et al., Biochem. Biophys. Res. Commun. 287:493-500. 2001) develop chylous ascites in some genetic backgrounds (Pennisi, et al., Nat. Genet. 24:434-437. 2000), suggesting that both proteins may be involved in the regulation of lymphatic development. Additionally, upregulation of VEGFR-3 expression by transcription factor Prox-1 has been shown to be involved in development of LEC (See PCT/US03/06900 and Petrova et al., EMBO J. 21:4593-4599, 2002).

Similar to the effects of Prox-1, it is contemplated that upregulation of the LEC-specific genes disclosed herein is useful in promoting LEC development as a treatment for LEC disorders characterized by an under-developed lymphatic system. Conversely, decreasing activity of LEC-specific genes and downregulating LEC development is contemplated as a treatment for LEC disorders characterized by an over-developed lymphatic system such as lymphedema. Additionally, use of the genes or gene products disclosed herein as prophylactic therapeutics designed to serve as a therapeutic antagonist in therapies involving other primary agents is also contemplated, such as the use of a material disclosed herein to counter the influence of the primary agent on LEC gene expression. It is known in the art that ex vivo transfection of cells and subsequent transfer of these cells to patients is an effective method to upregulate in vivo levels of the transferred gene and to provide relief from a disease resulting from under-expression of the gene(s) (Gelse et al., Arthritis Rheum. 48:430-41. 2003; Huard et al, Gene Ther. 9:1617-26.2002; Kim et al., Mol. Ther. 6:591-600 2002).

To develop a therapy for treating irregularities of LEC development, endothelial cells, such as CAECs, SAVECs, LECs or BECs, are isolated from individuals experiencing a LEC disorder (e.g. lymphedema) and then placed in an appropriate culture medium (see above) to promote the growth and viability of the cells. The cells are then transfected with a viral vector designed to express any one of the LEC-specific genes to initiate LEC differentiation of the non-LECs in vitro and to promote growth of the LECs in culture. Transfection of cells with a viral vector, such as a replication-deficient adenovirus, adeno-associated virus, lentivurus or retrovirus, is well known to those of skill in the art (see, e.g., Laitinen et al., Hum. Gene Ther. 9:1481-1486, 1998; Petrova et al., supra)and is described above.

Cells transfected with a LEC-specific gene are then transferred into an affected patient in therapeutically effective numbers to promote LEC expansion in vivo. In preferred embodiments, the manipulated cells are autologous cells. These cells are delivered by one or more administrations typically involving injection. The cells are delivered at a local site of an LEC disease or disorder such as lymphedema; alternatively, the cells are delivered systemically, e.g., by injection remote to the perceived site in need of such cells or their product(s).

Administration of transfected cells to patients with lymphedema provides supplementary LECs that are incorporated into the lymphatic network, thereby promoting lymphatic development and lymph clearance to relieve the symptoms of lymphedema. It is contemplated that a method comprising LEC-specific gene transfection into endothelial cells and administration of transfected cells is useful in the treatment of any disease characterized by an alteration in LEC numbers or activity, such as lymphedema, lymphangioma, lymphangiomyeloma, lymphangiomatosis, lymphangiectasis, lymphosarcoma, and lymphangiosclerosis. Additionally, such methods are useful in ameliorating a symptom (e.g., lymph-induced swelling in the case of lymphedema) associated with such diseases.

### Example 4

### MICROARRAY ANALYSIS TO DETECT LEC-AND LYMPH-RELATED DISORDERS

All LEC-specific genes identified, specifically any gene found in any of Tables 1 and 2 herein, are contemplated to be useful in the detection of LEC in vivo and in determining the extent of the lymphatic vasculature in a sample. The LEC-specific genes are also expected to be useful in diagnosing lymphedema and other LEC-related disorders. Further, the invention comprehends the use of a plurality of LEC-specific genes or their gene products for any of the applications identified herein. In this latter context, it is further contemplated that one or more LEC-specific genes, or their gene products, may be used in combination (regardless of whether such materials are physically or temporally combined or not) with at least one of the LEC-specific genes, or gene products, identified in Tables 3 and/or 4 of PCT/US03/06900.

Another aspect of the invention is a composition comprising a plurality of polynucleotide probes for use in detecting gene expression pattern(s) characteristic of particular cell type(s) and for detecting changes in the expression pattern of a particular cell type, e.g., lymphatic endothelial cells. The term “polynucleotide probe” is used herein to refer to any one of the nucleic acid sequences listed in SEQ ID NOS: 5, 7, 9, 11, 13, 15, 17, 19, 21, 23, 25, 27, 29, 31, 33, 35, 37, 39 and 41, or any fragment thereof or a nucleic acid having a sequence encoding an amino acid sequence set forth in SEQ ID NOS: 6, 8, 10, 12, 14, 16, 18, 20, 22, 24, 26, 28, 30, 32, 34, 36, 38, 40, 42, 43, 44, 45, and 46, or a fragment thereof. Preferably, the fragment is at least 10 nucleotides in length; more preferably, it is at least 20 nucleotides in length. Such a composition is employed for the diagnosis and/or treatment of any condition or disease in which the dysfunction or non-function of lymphatic endothelial cells is implicated or suspected. The composition may alternatively include any one of genes found in Tables 3 and 4 of PCT/US03/06900 (specifically incorporated herein by reference). In one embodiment, the present invention provides a composition comprising a plurality of polynucleotide probes, wherein at least a subset of the polynucleotide probes comprises at least a portion of an expressed gene isolated from a population of LEC-specific genes identified above. Also contemplated is a composition comprising a plurality of polynucleotide probes, with at least a subset of such probes each comprising a unique sequence selected from the group consisting of SEQ ID NOS: 5, 7, 9, 11, 13, 15, 17, 19, 21, 23, 25, 27, 29, 31, 33, 35, 37, 39, 41, and those sequences identified in any one of Tables 3 and 4 of PCT/US03/06900. Preferably, the composition comprises a subset of at least 3 polynucleotides, each having a different sequence selected from the group consisting of SEQ ID NOS: 5, 7, 9, 11, 13, 15, 17, 19, 21, 23, 25, 27, 29, 31, 33, 35, 37, 39, 41, and those sequences identified in any one of Tables 3 and 4 of PCT/US03/06900. Also preferred are compositions comprising at least 5, at least 7, at least 9, at least 15, at least 20, or at least 25 distinct polynucleotides having distinct sequences selected from the group of SEQ ID NOS: 5, 7, 9, 11, 13, 15, 17, 19, 21, 23, 25, 27, 29, 31, 33, 35, 37, 39, 41, and those identified in any one of Tables 3 and 4 of PCT/US03/06900.

The composition is particularly useful as a set of hybridizable array elements in a microarray for monitoring the expression of a plurality of target polynucleotides. The microarray comprises a substrate and the hybridizable array elements. The microarray is used, for example, in the diagnosis and/or prognosis of a disease derived from aberrant lymphatic endothelial cell activity, such as lymphedema, lymphangioma, lymphangiomyeloma, lymphangiomatosis, lymphangiectasis, lymphosarcoma, and lymphangiosclerosis. Compositions may be useful in identifying more than one cell type and may be useful in the diagnosis and/or prognosis of more than one disease, disorder or condition. Further, useful information is obtained from those probes yielding a signal and from those probes not yielding a signal.

A polynucleotide comprising the sequence of any one of SEQ ID NOS: 5, 7, 9, 11, 13, 15, 17, 19, 21, 23, 25, 27, 29, 31, 33, 35, 37, 39, 41, and any one of the genes found in any of Tables 3 and 4 of PCT/US03/06900 may be used for the diagnosis of a condition, disorder, or disease in which the abnormal expression of any one of the genes encoded by SEQ ID NOS: 5, 7, 9, 11, 13, 15, 17, 19, 21, 23, 25, 27, 29, 31, 33, 35, 37, 39, 41, and any gene found in any one of Tables 3 and 4 of PCT/US03/06900, is associated. For example, a polynucleotide comprising any one of the sequences set forth in SEQ ID NOS: 5, 7, 9, 11, 13, 15, 17, 19, 21, 23, 25, 27, 29, 31, 33, 35, 37, 39, 41, and a gene found in any of Tables 3 and 4 of PCT/US03/06900 may be used in hybridization or PCR assays of a fluid or tissue (e.g., obtained from a biopsy) to detect abnormal gene expression in patients with lymphedema or another lymph-associated disease, or suspected of having such a disease. In addition, a polynucleotide comprising a sequence encoding any of the amino acid sequences set forth in SEQ ID NOS: 6, 8, 10, 12, 14, 16, 18, 20, 22, 24, 26, 28, 30, 32, 34, 36, 38, 40, 42, 43, 44, 45, and 46, used in conjunction with at least one gene found in any of Tables 3 and 4 of PCT/US03/06900 is useful for the diagnosis of conditions or diseases associated with aberrant expression of a polypeptide having any one of those amino acid sequences. Fragments comprising at least 10 nucleotides are also useful in these diagnostic methods.

Expression profiles are generated using the compositions of the invention comprising, e.g., at least one sequence as set forth in SEQ ID NOS: 5, 7, 9, 11, 13, 15, 17, 19, 21, 23, 25, 27, 29, 31, 33, 35, 37, 39 or 41. The expression profile generated from a microarray is used to detect changes in the expression of genes implicated in disease.

### Example 5

### COMPOSITIONS OF THE INVENTION USEFUL FOR MODULATING VEGF-C/VEGFR-3INTERACTIONS AND LEC DEVELOPMENT

VEGF-C and VEGF-D knockout mice demonstrate aberrant vascular development that can be overcome by administration of exogenous VEGF-C and/or VEGF-D polypeptide. To determine if transcriptional regulation by any one of the proteins having an amino acid sequence set forth in SEQ ID NOS: 18, 26, 28, 30, 32, 34, 36, 38 or 40 (which may act as LEC-specific transcription factors) can overcome this vascular defect due to its potential interaction with, and transcriptional effect on, the VEGFR-3 promoter, VEGF-C or VEGF-D knockout mice are genetically crossed by interbreeding with mice overexpressing a polypeptide having an amino acid sequence of any one of SEQ ID NOS: 18, 26, 28, 30, 32, 34, 36, 38, 40, 44, 45 or 46 from a cell-specific-promoter (e.g., a K-14 keratin promoter). The effects of transcription factor (TF) activity on lymphedema are assessed through measurement of lymphedema and vascular development, as described in Karkkainen et al. (Proc. Natl. Acad. Sci. USA. 98:12677-12682.2001).

Survival of the knockout mice and detection of lymphatic development in the VEGF-C and/or VEGF-D knockout/TF-overexpressing mice indicates that the transcription factor of interest induces VEGFR-3 signaling and plays a role in lymphangiogenesis.

VEGF-C overexpressing mice (K-14-VEGF-C Tg) exhibit an extensive network of lymphatic vasculature, are prone to tumor metastasis, and demonstrate upregulated VEGFR-3 expression and symptoms of lymphedema (U.S. Pat. No. 6,361,946). To determine if the LEC-specific genes identified herein aid in regulating VEGF-C signaling through VEGFR-3, K-14-VEGF-C Tg mice are administered a composition of the invention, comprising at least one LEC-specific polypeptide or polypeptide fragment. Particularly useful are those fragments from extracellular proteins having sequences set forth in SEQ ID NOS: 6, 8, 10, 12, 14, 16, 20, 22, 24, 42 and 43.

Decreased lymphangiogenesis, decreased incidence of tumor metastasis, and decreased levels of VEGFR-3 exhibited by the LEC-specific-composition-treated K-14-VEGF-C mutant animals, as compared to the K-14-VEGF-C Tg single mutant animal, indicates that the administered composition interferes with VEGF-C signaling through VEGFR-3.

A result indicating that the composition of the invention is a negative regulator of lymphangiogenesis identifies a composition useful in a method of treating disorders mediated by extensive lymphatic vasculature, such as lymphangiogenesis in tumor development or lymphangiosarcoma Such a composition, comprising at least one LEC-specific polypeptide, may be administered to a patient or an animal in need, thereby inhibiting or preventing the induction of lymphangiogenesis by interfering with lymphangiogenic signaling. A result indicating that a composition of the invention is a positive regulator of lymphatic endothelial cells identifies a composition suitable for use in a method of treating disorders characterized by insufficient LEC development, e.g., insufficient development of a lymphatic network.

Additionally, to determine the effects of the invention in vivo, compositions of the invention are administered to animals affected by lymphedema in the Chy mouse model of lymphedema (Lyon et al., Mouse News Lett. 71: 26, 1984). Lymphedema symptoms, such as footpad swelling and fluid retention/ascites, are measured after treatment with either a soluble LEC-specific polypeptide, as described above, or with a LEC-specific gene therapy vector designed to regulate either cell surface proteins or intracellular proteins, comprising at least one of SEQ ID NOS: 5, 7, 9, 11, 13, 15, 17, 19, 21, 23, 25, 27, 29, 31, 33, 35, 37, 39 or 41, as disclosed previously. Improvement of lymphedema in the Chy mouse model after administration of compositions of the invention indicate that a composition which affects LEC-specific genes is capable of regulating LEC disorders, and is expected to be a useful therapeutic in the treatment of human lymphatic disorders.

Additionally contemplated is the treatment of subjects affected by lymphedema with a combination of therapeutics that includes a composition of the invention in conjunction with a lymphatic growth factor such as VEGF-C and/or VEGF-D to overcome impaired lymphatic vascular development.

From the foregoing it will be appreciated that, although specific embodiments of the invention have been described herein for purposes of illustration, various modifications may be made without deviating from the spirit and scope of the invention.

All of the above-cited U.S. patents, U.S. patent application publications, U.S. patent applications, foreign patents, foreign patent applications and non-patent publications referred to in this specification are incorporated herein by reference in their entireties.

1. A method for differentially modulating the growth or differentiation of blood endothelial cells (BEC) or lymphatic endothelial cells (LEC), comprising contacting endothelial cells with a composition comprising an agent that differentially modulates blood or lymphatic endothelial cells, said agent selected from the group consisting of:
(a) a polypeptide that comprises an amino acid sequence of a BEC polypeptide or a LEC polypeptide, or an active fragment of said polypeptide;
(b) a polynucleotide that comprises a nucleotide sequence that encodes a polypeptide according to (a);
(c) an antibody that specifically binds to a polypeptide according to (a);
(d) a polypeptide comprising a fragment of the antibody of (c), wherein the fragment and the antibody bind to the polypeptide;
(e) an antisense nucleic acid to a human gene or mRNA encoding the polypeptide of (a);
(f) an interfering RNA (RNAi) to a human gene or mRNA encoding the polypeptide of (a).

2. The method according to claim 1, wherein the endothelial cells are contacted with the composition ex vivo.

3. The method according to claim 1, wherein the composition comprises a pharmaceutically acceptable diluent, adjuvant, or carrier, and the contacting step comprises administering the composition to a mammalian subject to differentially modulate BECs or LECs in the mammalian subject.

4. The method according to claim 3, comprising:
identifying a human subject with a disorder characterized by hyperproliferation of LECs; and
administering to the human subject the composition, wherein the agent differentially inhibits LEC growth compared to BEC growth.

5. The method according to claim 3, comprising:
identifying a human subject with a disorder characterized by hyperproliferation of LECs;
screening LECs of the subject to identify overexpression of a polypeptide set forth in any of Tables 1 and 2 herein; and
administering to the human subject the composition, wherein the agent differentially inhibits LEC growth compared to BEC growth by inhibiting expression of the polypeptide identified by the screening step.

6. The method according to claim 3 of modulating the growth of lymphatic endothelial cells in a human subject, comprising steps of:
identifying a human subject with a hypoproliferative lymphatic disorder;
screening the subject to identify underexpression or underactivity of a LEC polypeptide set forth in any of Tables 1 and 2 herein;
administering to the human subject said composition, wherein the agent comprises the LEC polypeptide (a) identified by the screening step or an active fragment of said polypeptide, or comprises the polynucleotide (b) that comprises a nucleotide sequence that encodes the polypeptide.

7. (canceled)

8. The method according to claim 1, wherein the polypeptide is a LEC polypeptide selected from the LEC polypeptides set forth in any of Tables 1 and 2 herein, and the agent differentially modulates LEC growth or differentiation over BEC growth or differentiation.

9. The method according to claim 1, wherein the polypeptide is a BEC polypeptide selected from the BEC polypeptides set forth in Tables 1 and 2 herein, and the agent differentially modulates BEC growth or differentiation over LEC growth or differentiation.

10. The method according to any of claims 5 or 6, further comprising screening LECs of the subject to identify overexpression of a second polypeptide set forth in any of Tables 3 and 4 of PCT/US03/06900.

11. The method or use according to claim 8, wherein the LEC polypeptide comprises an amino acid sequence selected from the group consisting of SEQ ID NOs: 6, 8, 10, 12, 14, 16, 20, 22, 24, 42 and 43.

12-15. (canceled)

16. A method of treating hereditary lymphedema comprising:
identifying a human subject with lymphedema and with a mutation in at least one allele of a gene encoding a LEC protein identified in any of Tables 1 and 2, wherein the mutation correlates with lymphedema in human subjects, and with the proviso that said LEC protein is not VEGFR-3; and
administering to said subject a composition comprising a lymphatic growth agent selected from the group consisting of VEGF-C polypeptides, VEGF-D polypeptides, VEGF-C polynucleotides, and VEGF-D polynucleotides.

17. (canceled)

18. A method of screening for an endothelial cell disorder or predisposition to said disorder, comprising:
obtaining a biological sample containing endothelial cell mRNA from a human subject; and
measuring expression of a BEC or LEC gene from the amount of mRNA in the sample transcribed from said gene, wherein the BEC or LEC gene encodes a polypeptide identified in any of Tables 1 and 2.

19. A method of monitoring the efficacy or toxicity of a drug on endothelial cells, comprising the steps of:
measuring expression of at least one BEC or LEC gene in endothelial cells of a mammalian subject before and after administering a drug to the subject, wherein the at least one BEC or LEC gene encodes a polypeptide set forth in any of Tables 1 and 2, and wherein changes in expression of the BEC or LEC gene correlates with efficacy or toxicity of the drug on endothelial cells.

20. A method of identifying compounds that modulate growth of endothelial cells, comprising
culturing endothelial cells in the presence and absence of a compound; and
measuring expression of at least one BEC or LEC gene in the cells, wherein the BEC or LEC gene is selected from the genes encoding polypeptides set forth in any of Tables 1 and 2, wherein a change in expression of at least one BEC gene in the presence compared to the absence of the compound identifies the compound as a modulator of BEC growth, and wherein a change in expression of at least one LEC gene in the presence compared to the absence of the compound identifies the compound as a modulator of LEC growth.

21. The method according to claim 20 of screening for a compound that selectively modulates BEC or LEC growth or differentiation,
wherein the measuring step comprises measuring expression of at least one BEC gene and at least one LEC gene in the cells, and
wherein the method comprises screening for a compound that selectively modulates BEC or LEC growth or differentiation by selecting a compound that differentially modulates expression of the at least one BEC gene compared to expression of the at least one LEC gene.

22. A composition comprising
an isolated polynucleotide that comprises a nucleotide sequence that encodes a polypeptide comprising an amino acid sequence selected from the group consisting of SEQ ID NOs: 6, 8, 10, 12, 14, 16, 18, 20, 22, 24, 26, 28, 30, 32, 34, 36, 38, 40, 42, 43, 44, 45, and 46; and
a pharmaceutically acceptable diluent, carrier or adjuvant.

23. The composition according to claim 22, comprising a polynucleotide that comprises a nucleotide sequence selected from the group consisting of SEQ ID NOS: 5, 7, 9, 11, 13, 15, 17, 19, 21, 23, 25, 27, 29, 31, 33, 35, 37, 39 and 41, or a fragment thereof that encodes the polypeptide.

24. An expression vector comprising an expression control sequence operably linked to a polynucleotide that comprises a nucleotide sequence that encodes a polypeptide comprising an amino acid sequence selected from the group consisting of SEQ ID NOs: 6, 8, 10, 12, 14, 16, 18, 20, 22, 24, 26, 28, 30, 32, 34, 36, 38, 40, 42, 43, 44, 45, and 46.

25. The expression vector according to claim 24 that is a replication-deficient adenoviral or adeno-associated viral vector containing the polynucleotide.

26-27. (canceled)

28. A host cell transformed or transfected with an expression vector according to claim 24.

29. A method for producing a LEC polypeptide comprising the steps of growing a host cell according to claim 28 under conditions in which the cell expresses the polypeptide encoded by the polynucleotide.

30. A purified and isolated polypeptide comprising an amino acid sequence selected from the group consisting of
(a) SEQ ID NOS: 6, 8, 10, 12, 14, 16, 18, 20, 22, 24, 26, 28, 30, 32, 34, 36, 38, 40, 42, 43, 44, 45, and 46; and
(b) an extracellular domain fragment of at least 10 amino acids of an amino acid sequence of SEQ ID NOS: 6, 8, 10, 12, 14, 16, 20, 22, 24, 42 and 43.

31-33. (canceled)

34. A fusion protein comprising a polypeptide according to claim 30 fused to an immunoglobulin fragment comprising an immunoglobulin constant region.

35. A composition comprising a polypeptide or protein according to claim 30 and a pharmaceutically acceptable diluent, carrier or adjuvant.

36. (canceled)

37. An antibody or antigen binding fragment thereof that specifically binds to a polypeptide according to claim 30.

38-39. (canceled)

40. A method of identifying a LEC nucleic acid comprising:
(a) contacting a biological sample containing a candidate LEC nucleic acid with a polynucleotide comprising a fragment of at least 14 contiguous nucleotides of a sequence selected from the group consisting of SEQ ID NOS: 5, 7, 9, 11, 13, 15, 17, 19, 21, 23, 25, 27, 29, 31, 33, 35, 37, 39 and 41, or a complement thereof, under the following stringent hybridization conditions:
(i) hybridization at 42° C. for 20 hours in a solution containing 50% formamide, 5×SSPE, 5× Denhardt's solution, 0.1% SDS and 0.1 mg/ml denatured salmon sperm DNA, and
(ii) washing for 30 minutes at 65° C. in 1×SSC, 0.1% SDS; and
(b) detecting hybridization of said candidate LEC nucleic acid and said polynucleotide, thereby identifying a LEC nucleic acid.

41. A method of identifying a LEC protein comprising:
(a) contacting a biological sample containing a candidate LEC protein with a LEC protein binding partner selected from the group consisting of an antibody according to claim 37 or a protein according to claim 39, under conditions suitable for binding therebetween; and
(b) detecting binding between said candidate LEC protein and said LEC binding partner, thereby identifying a LEC protein.

42. A method of identifying a LEC comprising:
(a) contacting a biological sample comprising cells with a LEC binding partner under conditions suitable for binding therebetween, wherein said LEC binding partner comprises an antibody that binds to a polypeptide comprising a sequence selected from the group consisting of SEQ ID NOS: 6, 8, 10, 12, 14, 16, 20, 22, 24, 42 and 43, or comprises an antigen binding fragment of said antibody; and
(b) identifying a LEC by detecting binding between a cell and said LEC binding partner, where binding of the LEC binding partner to the cell identifies a LEC.

43. A method of assaying for risk of developing hereditary lymphedema, comprising
(a) assaying nucleic acid of a human subject for a mutation that correlates with a hereditary lymphedema phenotype and alters the encoded amino acid sequence of at least one gene allele of the human subject when compared to the amino acid sequence of the polypeptide encoded by a corresponding wild-type gene allele, wherein the wild-type polypeptide is a polypeptide identified in any of Tables 1 and 2.

44. A method of assaying for risk of developing hereditary lymphedema, comprising
(a) assaying nucleic acid of a human subject for a mutation that correlates with a hereditary lymphedema phenotype and alters the encoded amino acid sequence of at least one gene allele, at least one transcription factor allele or at least one LEC gene allele encoding a wild-type adhesion polypeptide of the human subject when compared to the amino acid sequence of the polypeptide encoded by a corresponding wild-type gene allele,
(i) wherein the wild-type polypeptide comprises an amino acid sequence selected from the group consisting of SEQ ID NOS: 6, 8, 10, 12, 14, 16, 20, 22, 24, 42, and 43;
(ii) wherein the wild-type transcription factor polypeptide comprises an amino acid sequence selected from the group consisting of SEQ ID NO: 18, 26, 28, 30, 32, 34, 36, 38, 40, 44, 45, and 46; and
(iii) wherein the wild-type adhesion polypeptide comprises an amino acid sequence selected from the group consisting of SEQ ID NO: 20 and SEQ ID NO: 43; and
(b) correlating the presence or absence of said mutation in the nucleic acid to a risk of developing hereditary lymphedema, wherein the presence of said mutation in the nucleic acid correlates with an increased risk of developing hereditary lymphedema, and wherein the absence of said mutation in the nucleic acid correlates with no increased risk of developing hereditary lymphedema.

45. (canceled)

46. The method according to claim 44 further comprising assaying said nucleic acid for a mutation that alters the expression of Sox18.

47-54. (canceled)

55. The method according to claim 44 wherein said method comprises at least one procedure selected from the group consisting of:
(a) determining a nucleotide sequence of at least one codon of at least one polynucleotide of the human subject;
(b) performing a hybridization assay to determine whether nucleic acid from the human subject has a nucleotide sequence identical to or different from one or more reference sequences;
(c) performing a polynucleotide migration assay to determine whether nucleic acid from the human subject has a nucleotide sequence identical to or different from one or more reference sequences; and
(d) performing a restriction endonuclease digestion to determine whether nucleic acid from the human subject has a nucleotide sequence identical to or different from one or more reference sequences.

56. (canceled)

57. A method of screening for a hereditary lymphedema genotype in a human subject, comprising:
(a) providing a biological sample comprising nucleic acid from said subject, and
(b) analyzing said nucleic acid for the presence of a mutation altering the encoded amino acid sequence of the at least one allele of at least one gene in the human subject relative to a human gene encoding an amino acid sequence selected from the group consisting of SEQ ID NOs: 6, 8, 10, 12, 14, 16, 18, 20, 22, 24, 26, 28, 30, 32, 34, 36, 38 40, 42, 43, 44, 45, and 46, wherein the presence of a mutation altering the encoded amino acid sequence in the human subject in a manner that correlates with lymphedema in human subjects identifies a hereditary lymphedema genotype.

58-60. (canceled)

61. A method of inhibiting lymphangiogenesis comprising
administering to a subject an inhibitor of a LEC transmembrane polypeptide,
wherein the LEC transmembrane polypeptide comprises an amino acid sequence selected from the group consisting of SEQ ID NOs: 6, 8, 10, 12, 14, 16, 20, 22, 24, 42 and 43; and
wherein the inhibitor is selected from the group consisting of
(a) a soluble extracellular domain fragment of the LEC transmembrane polypeptide;
(b) an antibody that binds to the extracellular domain of the LEC transmembrane polypeptide;
(c) a polypeptide comprising an antigen binding domain of the antibody according to (b); and
(d) an antisense nucleic acid complementary to the nucleic acid encoding the LEC transmembrane polypeptide or its complement.

62. The method according to claim 61, wherein the inhibitor is a polypeptide comprising an extracellular domain fragment of an LEC polypeptide, wherein the sequence of said extracellular domain is selected from the group consisting of amino acids 1-1005 of SEQ ID NO:43.

63. (canceled)

64. A method for modulating lymphangiogenesis in a mammalian subject comprising: administering to a mammalian subject in need of modulation of lymphangiogenesis an antisense molecule to a LEC polynucleotide, in an amount effective to inhibit transcription or translation of the polypeptide encoded by the LEC polynucleotide, wherein the LEC polynucleotide comprises a nucleotide sequence selected from the group consisting of SEQ ID NOS: 5, 7, 9, 11, 13, 15, 17, 19, 21, 23, 25, 27, 29, 31, 33, 35, 37, 39 and 41.

65. A method of treating hereditary lymphedema, comprising:
(a) identifying a human subject with hereditary lymphedema and with a mutation that alters the encoded amino acid sequence of at least one polypeptide of the human subject, relative to the amino acid sequence of a polypeptide comprising an amino acid sequence selected from the group consisting of SEQ ID NOS: 6, 8, 10, 12, 14, 16, 20, 22, 24, 42 and 43; and
(b) administering to said subject a lymphatic growth factor selected from the group consisting of a VEGF-C polynucleotide, a VEGF-C polypeptide, a VEGF-D polynucleotide, and a VEGF-D polypeptide.

66. The method according to claim 8, wherein the agent further comprises a second LEC polypeptide selected from the LEC polypeptides set forth in any of Tables 3 and 4 of PCT/US03/06900.

67-68. (canceled)

69. The method according to claim 43, further comprising assaying the nucleic acid for a second mutation that correlates with a hereditary lymphedema phenotype and alters the encoded amino acid sequence of a second gene allele of the human subject when compared to the amino acid sequence of the polypeptide encoded by a corresponding wild-type allele of said second gene, wherein the wild-type polypeptide encoded by said second gene is a polypeptide identified in any of Tables 3 and 4 of PCT/US03/06900.

70. An isolated polypeptide comprising an amino acid sequence at least 95% identical to amino acids 1-1005 of SEQ ID NO: 43.

71. A soluble polypeptide comprising a fragment of the amino acid sequence set forth in SEQ ID NO: 43, wherein said fragment lacks the transmembrane and intracellular amino acids from approximately residues 1006-1028 of SEQ ID NO: 43.

72. An isolated polypeptide comprising a polypeptide selected from the group consisting of:
a) an adhesion domain having an amino acid sequence at least 95% identical to a fragment of a polypeptide comprising the amino acid sequence set forth in SEQ ID NO: 20 or SEQ ID NO: 43, wherein said fragment includes at least one vonWillebrand factor domain;
b) a Cache domain and comprising an amino acid sequence at least 95% identical to a fragment of a polypeptide comprising the amino acid sequence set forth in SEQ ID NO: 20 or SEQ ID NO: 43, wherein said fragment includes at least one Cache domain;
c) an amino acid sequence at least 95% identical to a protein kinase domain selected from the group consisting of a protein kinase domain set out in SEQ ID NO: 8, in SEQ ID NO: 26, at approximately amino acids 88-342 of SEQ ID NO: 42 and at approximately amino acids 51-317 of SEQ ID NO: 46;
d) an amino acid sequence at least 95% identical to a Zinc binding domain selected from the group consisting of a Zinc-binding domain set out in SEQ ID NO: 36, a Zinc-binding domain at approximately amino acids 527-561 of SEQ ID NO: 6, and a Zinc-binding domain at approximately amino acids 1030-1094 of SEQ ID NO: 45;
e) an amino acid sequence at least 95% identical to a transcription factor selected from the group consisting of SEQ ID NO: 30, SEQ ID NO: 38, SEQ ID NO: 40, and SEQ ID NO: 44:
f) an amino acid sequence at least 95% identical to a cytoskeleton regulation protein selected from the group consisting of SEQ ID NO: 18, SEQ ID NO: 36, and SEQ ID NO: 45;
g) a SPRY domain having an amino acid sequence at least 95% identical to a fragment of a polypeptide comprising the amino acid sequence set forth in SEQ ID NO: 6, wherein said fragment includes at least one SPRY domain;
h) a cation transport activity and comprising an amino acid sequence at least 95% identical to a fragment of a polypeptide comprising the amino acid sequence set forth in SEQ ID NO: 12, wherein said fragment includes at least one cation transport domain;
i) a guanine-nucleotide dissociation stimulator activity and comprising an amino acid sequence at least 95% identical to a fragment of a polypeptide comprising the amino acid sequence set forth in SEQ ID NO: 28, wherein said fragment includes at least one RCC1-like domain;
j) a ubitquitin ligase activity and comprising an amino acid sequence at least 95% identical to a fragment of a polypeptide comprising the amino acid sequence set forth in SEQ ID NO: 28, wherein said fragment includes at least one Hect domain;
k) a Kelch domain and comprising an amino acid sequence at least 95% identical to a fragment of a polypeptide comprising the amino acid sequence set forth in SEQ ID NO: 30 or SEQ ID NO: 44, wherein said fragment includes at least one Kelch domain; and,
l) a GTP-binding activity and comprising an amino acid sequence at least 95% identical to a fragment of a polypeptide comprising the amino acid sequence set forth in SEQ ID NO: 32, wherein said fragment includes at least one GTP-binding domain.

73. (canceled)

74. A fusion protein comprising a polypeptide according to any one of claims 70-72 and a heterologous polypeptide.

75. An antibody that specifically binds to a polypeptide according to any one of claims 70-72.

76-85. (canceled)

86. A polynucleotide comprising a nucleotide sequence that encodes a polypeptide according to any one of claims 70-72.

87. An expression vector comprising a polynucleotide according to claim 86 or 90 operatively linked to an expression control sequence.

88. An expression vector according to claim 87 that is a replication-deficient adenoviral vector or adeno-associated virus vector.

89. (canceled)

90. A polynucleotide comprising a nucleotide sequence that encodes a polypeptide according to claim 74.
